(19) United States
(12) Patent Application Publication
(10) Pub. No.: US 2014/0023592 A1
(43) Pub. Date: Jan. 23, 2014

(54) SELF COUPLING RECOMBINANT ANTIBODY FUSION PROTEINS

(71) Applicants: RWTH Aachen, Aachen (DE); Fraunhofer-Gesellschaft zur Forderung der Angewandten Forschung e.V., Munchen (DE)

(72) Inventors: Markus Ribbert, Biberach an der Riss (DE); Stefan Barth, Aachen (DE); Florian Kampmeier, Aachen (DE)

(73) Assignees: RWTH Aachen, Aachen (DE); Fraunhofer-Gesellschaft zur Forderung der Angewandten Forschung e.V., Munchen (DE)

(21) Appl. No.: 14/041,966

(22) Filed: Sep. 30, 2013

Related U.S. Application Data
(63) Continuation of application No. 12/670,565, filed on Mar. 12, 2010.

(30) Foreign Application Priority Data
Jul. 25, 2007 (DE) 102007035160.9

(51) Int. Cl. C12N 9/10; A61K 49/00; C07K 14/705; C07K 14/71; C07K 14/485; G01N 21/64; C07K 16/28

(52) U.S. Cl. CPC C12N 9/1085; G01N 21/6486; A61K 49/0056; C07K 16/2878; C07K 14/71; C07K 16/283; C07K 14/485; C07K 14/70578
USPC 424/9.6; 435/7.94; 435/188; 435/7.21

(57) ABSTRACT

A compound comprising three components A, B, and C, which components are covalently bound forming the compound having the structure A-B-C wherein
component A has a specific binding affinity for antigens,
component B is covalently linked to component A
component C is a compound having an alkylated purine or pyrimidine moiety such as guanine, cytosine or a Coenzyme A moiety and linked thereto a moiety having a physiological effect with the proviso that
component B has an catalytical or acceptor activity to couple component C with covalently coupled components A-B.

### CROSS-REFERENCE TO RELATED APPLICATIONS

This patent application is a continuation of co-pending U.S. patent application Ser. No. 12/670,565, which is a national stage filing of PCT Application No. PCT/EP2008/059831 filed Jul. 25, 2008, and which claims priority to German Patent Application No. 102007035160.9 filed Jul. 25, 2007, each of which are hereby incorporated herein by reference.

### TECHNICAL FIELD OF THE INVENTION

The present invention relates to a complex formed from at least one component A and at least one component B. The present invention also relates to nucleic acids and/or vectors coding for such a complex. Furthermore an integral part of the complex comprises a component C which consists of an orthogonale substrate for component B which is chemically linked to a chemical or solid matter. Component C is added in a covalent coupling reaction to B through a substrate specific manner thereby transferring its inherent physico-chemical properties to the complex ABC.

### INTRODUCTION TO THE INVENTION

Today there is a series of approved methods available for diagnosis and/or therapy of malignant disorders in man like cancer, chronic inflammatory diseases and allergy. The classical therapeutic approaches have, because of their relatively unselective nature (for example radio- and chemotherapy in cancer treatment), a lot of severe side effects.

In the field of diagnosis there exist a lot of new high resolution imaging technologies that render possible a very exact topographic localization of e.g. solid tumors (X-Ray/magnetic resonance imaging (MRI)). Despite the correct localization the tumor biology and physiology is of great importance for the kind of therapy being optimal.

Modern molecular biology approaches like antibody technology open new opportunities and way of diagnosis and therapy. As a selective component and fusion partner to therapeutics/diagnostics can target almost every desired cell/tissue specific marker. They also drastically improve the specificity of therapy and limit the incidence of false, false-positive/false-negative diagnosis.

Before their application as immunodiagnostic tool or as immunotherapeutic (e.g. immunotoxin) full length antibodies have to be modified with detectable agents or effector molecules. A gold standard is still the chemical methodology. However, the chemical modification of antibodies very often leads to complications like loss of binding activity or specificity. Chemical properties of the generic proteins like solubility are also affected negatively in some cases. Due to more and more sophisticated applications there is a strong demand for functional modificated antibodies.

A further development in this field is the genetic fusion of recombinant antibodies to effector molecules. Unfortunately each of the resulting fusion proteins is limited to one or a few applications. For each new application field the antibody has to be coupled to another suited effector molecule resulting in loborious proces optimizations for each case. Additionally this approach is limited to peptidic effector molecules.

An object of the invention is to avoid as far as possible the loss of binding activity and specificity due to chemical modification of full length antibodies in e.g. fusion proteins used for cell targeting.

A further object of the present invention is providing a compound which enables the skilled person to use a similar or same tool both for diagnosis and therapy of diseases. Still another object of the invention is providing a compound avoiding immunogenicity/strong side effects of immunotherapeutics.

Yet another object of the invention is to provide a missing link between classical therapeutic approaches and more specific new technologies

### SUMMARY OF THE INVENTION

The invention relates to novel compounds, in particular fusion proteins, comprising at least one antigen specific binding moiety and at least one enzyme type protein which reacts covalently with a specific substrate.

The objects of the invention are solved by a compound comprising three components A, B, and C, which components are covalently bound forming the compound having the structure A-B-C wherein
component A has a specific binding affinity for antigens,
component B is covalently linked to component A
component C is a compound having an alkylated purine or pyrimidine moiety such as guanine, cytosine or a Coenzyme A moiety and linked thereto a moiety having a physiological effect with the proviso that
component B has a catalytical or acceptor activity to couple component C with covalently coupled components A-B.

In one particular embodiment of the invention the compound can be rearded as a complex with the generic structure:

Antigen binding moiety (A)−enzyme type protein (B)−C

The compound of the invention is in particular a heterologous complex comprising at least one recombinant fusion protein comprising at least one specific binding component A in particular cell-specific binding component and one enzyme type protein B and at least one additional component C that is covalently coupled to B.

In another embodiment of the invention the compound is a heterologous complex comprising at least one recombinant fusion protein comprising at least one component A binding to a soluble antigen and one enzyme type protein B and at least one additional component C that is covalently coupled to B.

In a specific embodiment, the compound of the invention has a covalent modification of component A with component C through component B.

In a further embodiment of the invention component A of the compound of the invention is a chemical moiety having a polypeptidic antigen binding structure and component B is an enzyme type protein linked to component A.

In yet another embodiment, component B of the compound of the invention is capable of reacting with component C in a substrate specific manner, thereby connecting covalently the complex AB with component C.

In particular, component A of the compound of the invention belongs to the group of antigen binding polypeptides/proteins targeting celltype specific markers, in particular component A is directed against disease specific structures of pathogenic substances or pathogenic matter. Some representatives of component A comprise moieties which are affinity moieties from affinity substances or affinity substances in their entirety selected from the group consisting of antibodies, receptor/receptor ligands, including protein A/IgG, avidin/biotin and the like, enzyme substrates, lectins, interleukins, cytokines, chemokines, lymphokines, allergens, peptidic allergens, recombinant allergens, allergen-idiotypical antibodies, autoimmune-provoking structures, tissue-rejection-inducing structures, immunoglobulin constant regions and their derivatives, mutants or combinations thereof. Furthermore component A can bind to soluble markers of disease/environment/food and feed safety or biodefense (e.g. toxins).

Component A may have a specific binding affinity also to antigens, which are immunologically relevant only when coupled to immunogenic molecules. These compounds typically addressed as haptenes are for instance low molecular substances, which—as individuals—are not provoking an immune response, but when inked to an immunogenic compound such as a protein.

In an embodiment of the invention the component B of the compound of the invention is a polypeptide that reacts covalently with a specific substrate. In particular, component B may be a derivative of the human DNA repair protein O⁶-alkylguanine-DNA alkyltransferase (AGT). The component B can be derived from the Acyl Carrier Protein (ACP). A person skilled in the art recognizes that there may be multiple alterations and modifications on the DNA or the amino acid level which lead to components with functional equivalence.

In a specific embodiment of the invention the substrate for component B consists of O6-benzylguanine, O2-benzylcytosine or a coenzyme A (CoA).

Advantageously in the compound of the invention components A-B are covalently coupled polypeptides.

Component C holds physico-chemical or physiological properties to be transferred to the complex AB.

In an embodiment of the invention component C of the compound of the invention is a drug, a detectable label or other components mediating biological activity in a targeted cell or organism.

In another embodiment of the invention component C of the compound of the invention is a solid phase or a support.

In a further embodiment of the invention component C of the compound of the invention comprises a moiety which serves as a substrate for component B.

In particular, component C can have the structure

(X)ₙ₁-(Y)-(Z)ₙ₂

with X being a for component B specific substrate and n1 being one or more preferentially 1-3 and Z being a drug, a detectable label or other components mediating biological activity in a targeted cell or organism and n2 being 1 or more.

The structural element Y of component C may fullfill the following fuctions: a spacer mediating the desired flexibility between X and Z (and this way between B and C) ensuring the functionality of each component within the assembled complex.

Further the linker structural element may contain structures enabling the controlled release of Z under certain environmental conditions during interactions like chemical reactions (e.g. pH sensitive or reducable structures for release in endosomes or the cytosol).

The linker structural element Y may also have linear, branched, tree like or polymeric structure.

Subject matter of the invention is also a nucleic acid molecule coding for polypeptides of the invention.

In an additional embodiment of the present invention there are provided expression cassettes comprising a polynucleotide encoding the polypeptide, in particular a chimeric polypeptide, comprising components A and B in the order AB or BA. Further different versions are possible: AAB, ABB, AAAB, AAAAB, BAA; BBA, BAAA, BAAAA, BAB and ABA.

The nucleic acid molecule of the invention and expression cassette of the invention may further be a part of a vector or vector system suitable for expression of the complexes AB (BA) in a host cell. Therefor also the vector is subject matter of the invention.

In a further embodiment there are provided methods for the expression of the recombinant genes encoding the recombinant compounds of the invention.

In a further embodiment the present invention provides for a method using a host cell comprising an afore mentioned expression vector of the invention and culturing the host cell under conditions suitable for the expression of the invention related complexes.

The host cell is further defined as a procaryotic host cell or a eucaryotic host cell like mammalian, plant or yeast cells.

Moreover the invention relates to methods of reacting a complex AB (BA) with a compound C comprising one ore more enzyme substrates for which B is specific and further carrying one or more copies of a drug, a detectable label or other components mediating biological activity in a targeted cell or organism.

Furthermore the invention relates to methods of preparing and administering the invention related complexes to cells in vitro and in vivo, with C carrying one or more copies of a drug, a detectable label or other components mediating biological activity in a targeted cell or organism.

Applications of the invention related complex include in vitro and in vivo diagnostic approaches in the field of human and animal disorders as well as analytic approaches in the field of environmental monitoring, ecotoxicology and biosensor applications, with C being or containing a detectable label.

In a certain embodiment of the afore mentioned applications the complex will be used in therapy for human and animal disorders, with C being or containing a drug or components mediating biological activity in a targeted cell or organism.

In a specific embodiment component A is directly immobilized via component C to a given surface (planar, bead) allowing to enrich the marker at a distinct location.

In another specific embodiment, component A is used to detect an enriched marker (pref. soluble, e.g. soluble CD30/CEA/PSA/sIL-2R/sFAS/sCD23/sCD26/sCD40L/sCD40/CRP/sVCAM-1/MCP1/thrombomodulin/plasma C4bBP/Protein C/activated proteinC/proteinS/von willebrand factor/TNFR/p55/p75/Fas (CD95)/Nerve growth factor R/CD27/CD30/Growth hormone R/GM-CSF/Erythropoietin-R/Thrombopoietin/G-CSF/IL-IRI/IL-IRII/IL-2Ra (Tac, CD25) IL-4R/IL-5Ra/IL-7R/IL/CNTFR/LIFR/Leptin R/IL-11R/IL-12/Stem cell factor R (c-kit)/Interferon R/Lipopolysaccharide R(CD14)/Complement receptor Type I/Hyaluronate R(CD44)/CD58/IgER (FceRII, CD23)/IgGR (FcgRII)/ICAM-1 (CD54)/ICAM-3 (CD50)/Transforming growth factor bRIII/Epidermal growth factor R (c-erb B)/Vascular endothelial growth factor R/Platelet derived growth factor R/Fibroblast growth factor/Colony stimulating factor-1R (MCFR, c-fms)/ARK/Tie/Insulin R/Insulin-like growth factor-IIR/mannose 6-phosphate R) at a distinct location (spot, bead) via component C having the following characteristics: optical including fluorescence, magnetic including resp. beads (e.g. FeOH-based), radiolabel including gamma ray emitting nuclides like Technetium-99m, Thallium-201, Gallium-67, Fluorine-18, Indium-111, ultrasound including resp. bubbles, electrochemical including enzymes like alkaline phosphatase, oligonucleotides like hybridization probes for PCR.

In vitro/in vivo detection of the distinct cells is via component C having the above-mentioned characteristics.

In an additional embodiment, the component A is binding to a cell surface marker being internalized (EGFR, CD30R, BCR, and the like); component C is an agent selected from the group of small molecules having cytotoxic/cytostatic activities.

In another specific embodiment, the component A is binding to a cell surface marker (MUC1, Syndecan1), not being internalized; component C is an agent delivering CpG motives, beta ray emitting nuclides like Iodine-131, Yttrium-90, Lutetium-177, or enzymes activating cytotoxic agents (directed enzyme prodrug therapy: DEPT using e.g. carboxypeptidase as enzyme).

In another embodiment component A contains or is composed of D-amino acids in an artificial process copying the above mentioned proteins/peptides which naturally are synthesized with L-amino acids.

In specific embodiments components A and B may be modified with or contain chemically modified azido and alkynyl monosaccharide precursors for labeling glycans, unnatural amino acids bearing azides and alkynes for residue-specific protein labeling or azido lipid substrates for probing lipidated proteins.

The process, called bioorthogonal labeling enables a site-specific modification of components A or B via click chemistry like described by Baskin, J. and Bertozzi, C. (2007).

### BRIEF DESCRIPTION OF THE DRAWINGS

FIG. 1 depicts Tris{[2-(tert butoxycarbonyl)ethoxy]methyl}methylamine;

FIG. 2 dpeicts N-Tris{[2-(tert-butoxycarbonyl)ethoxy]methyl}methyl trifluoroacetamide;

FIG. 3 depicts N-Tris{[2-(carboxy)ethoxy]methyl}methyl trifluoroacetamide;

FIG. 4 depicts N-Tris(BG-PEG4-NH-carbonylethyloxymethyl)methyl trifluoroacetamide;

FIG. 5 depicts Tris(BG-PEG4-NH-carbonylethyloxymethyl)methylamine;

FIG. 6 depicts N-Tris(BG-PEG4-NH-carbonylethyloxymethyl)methyl fluorescein-5-carboxamide;

FIG. 7 depicts the fluorescein-6-carboxamide corresponding to the embodiment of FIG. 6;

FIG. 8 depicts N-Tris(BG-PEG4-NH-carbonylethyloxymethyl)methyl chlorambucil-carboxamide;

FIG. 9 depicts N-Tris(BG-PEG4-NH-carbonylethyloxymethyl)methyl 5-maleimidopentane-carboxamide;

FIG. 10 depicts Tris(BG-PEG4-NH-carbonylethyloxymethyl)methyl 6-maleimido-hexanoic amide siRNA conjugate;

FIG. 11 depicts N-2-(2-(2-(2-azidoethoxy)ethoxy)ethoxy)ethyl-N′-tris[2-(tert-butoxy- [symbol] carbonyl)ethyl]methyl-urea;

FIG. 12 depicts 4-[2-carboxyethyl]-4-(2-(2-(2-(2-azidoethoxy)ethoxy)-ethoxy)ethylamino [symbol] carbonylamino)-1,7-heptanedicarboxylic acid;

FIG. 13 depicts N-Tris(BG-PEG4-NH-carbonylethyl)methyl-N′-2-(2-(2-(2-azidoethoxy) [symbol] ethoxy)ethoxy)ethyl-urea;

FIG. 14 depicts N-Tris-(BG-PEG4-NH-carbonylethyl)methyl-N′-2-(2-(2-(2-aminoethoxy) [symbol] ethoxy)ethoxy)ethyl-urea;

FIG. 15 depicts N-Tris-(BG-PEG4-NH-carbonylethyl)methyl-N′-2-(2-(2-(2-fluorescein-5-carboxamido-ethoxy)ethoxy)ethoxy)ethyl-urea;

FIG. 16 depicts the 6-fluorescein derivative corresponding to the embodiment of FIG. 15;

FIG. 17 depicts N-Tris(BG-PEG4-NH-carbonylethyl)methyl-N′-2-(2-(2-(2-chlorambucil-carboxamido-ethoxy)ethoxy)ethoxy)ethyl-urea;

FIG. 18 depicts N-Tris(BG-PEG4-NH-carbonylethyl)methyl-N′-2-(2-(2-(2-(6-maleimido-hexanoylamido)ethoxy)ethoxy)ethoxy)ethyl-urea;

FIG. 19 depicts N-Tris(BG-PEG4-NH-carbonylethyl)methyl-N′-2-(2-(2-(2-(6-maleimido [symbol] hexanoylamido)ethoxy)ethoxy)ethoxy)ethyl-urea siRNA conjugate;

FIG. 20 depicts Azido-PEG12-propionic acid 2-maleimidoethylamide;

FIG. 21 depicts Azido-PEG12-propionic acid 2-maleimidoethylamide CoA-SH conjugate;

FIG. 22 depicts Amino-PEG12-propionic acid 2-maleimidoethylamide CoA-SH conjugate;

FIG. 23 depicts N-Tris{2-(2-(2-(CoA-S-succinimido)ethylaminocarbonyl-PEG12)ethyl [symbol] aminocarbonyl)ethoxymethyl}methyl trifluoroacetamide;

FIG. 24 depicts N-Tris-{2-(2-(2-(CoA-S-succinimido)ethylaminocarbonyl-PEG12)ethyl [symbol] aminocarbonyl)ethoxymethyl}methylamine;

FIG. 25 depicts N-Tris{2-(2-(2-(CoA-S-succinimido)ethylaminocarbonyl-PEG12)ethyl [symbol] aminocarbonyl)ethoxymethyl}methyl fluorescein-5-carboxamide;

FIG. 26 depicts fluorescein-6-carboxamide corresponding to the embodiment of FIG. 25;

FIG. 27 depicts N-Tris-{2-(2-(2-(CoA-S-succinimido)ethylaminocarbonyl-PEG12)ethyl [symbol] aminocarbonyl)ethoxymethyl}methyl chlorambucil-carboxamide;

FIG. 28 depicts N-Tris{2-(2-(2-(CoA-S-succinimido)-ethyl-aminocarbonyl-PEG12)ethylamino-carbonyl)ethoxymethyl}methyl 6-maleimido-hexanoyl-amide;

FIG. 29 depicts N-Tris{2-(2-(2-(CoA-S-succinimido)ethylamino-carbonyl-PEG12)-ethylamino-carbonyl)ethoxymethyl}methyl 6-maleimidohexanoylamide siRNA conjugate;

FIG. 30 depicts N-Tris{2-(2-(2-(CoA-S-succinimido)ethylaminocarbonyl-PEG12)ethyl [symbol] aminocarbonyl)ethoxymethyl}methyl-N′-2-(2-(2-(2-azidoethoxy)-ethoxy) [symbol] ethoxy)ethyl-urea;

FIG. 31 depicts N-Tris-{2-(2-(2-(CoA-S-succinimido)ethylaminocarbonyl-PEG12)ethyl [symbol] aminocarbonyl)ethoxymethyl}methyl-N′-2-(2-(2-(2-aminoethoxy)-ethoxy) [symbol] ethoxy)ethyl-urea;

FIG. 32 depicts N-Tris{2-(2-(2-(CoA-S-succinimido)ethyl [symbol] aminocarbonyl-PEG12)ethyl [symbol] aminocarbonyl)ethoxymethyl}methyl-N′-2-(2-(2-(2-fluorescein-5-carbox [symbol] amidoethoxy)ethoxy)ethoxy)ethyl-urea;

FIG. 33 depicts a 6-fluorescein derivative corresponding to the embodiment of FIG. 32;

FIG. 34 depicts N-Tris{2-(2-(2-(CoA-S-succinimido)ethylaminocarbonyl-PEG12)ethyl [symbol] aminocarbonyl)ethoxymethyl}methyl-N′-2-(2-(2-(2-chlorambucil-carboxamido-ethoxy)ethoxy)ethoxy)ethyl-urea;

FIG. 35 depicts N-Tris-{2-(2-(2-(CoA-S-succinimido)ethylaminocarbonyl-PEG12)ethyl [symbol] aminocarbonyl)ethoxymethyl}methyl-N′-2-(2-(2-(2-(6-maleimido-hexanoyl [symbol] amido)ethoxy)ethoxy)ethoxy)ethyl-urea;

FIG. 36 depicts N-Tris{2-(2-(2-(CoA-S-succinimido)ethylaminocarbonyl-PEG12)ethyl [symbol] aminocarbonyl)ethoxymethyl}methyl-N′-2-(2-(2-(2-(6-maleimido-hexanoyl [symbol] amido)ethoxy)ethoxy)ethoxy)ethyl-urea siRNA conjugate;

FIG. 37 depicts a 5-Fluorescein-Lys-Fmoc-OH;

FIG. 38 depicts a 6-fluorescein-Lys-Fmoc-OH;

FIG. 39 depicts a 5-Fluorescein-Lys-OH;

FIG. 40 depicts a 6-fluorescein-Lys-OH;

FIG. 41 depicts a N-5-Fluorescein-N′-chlorambucil-Lys-OH;

FIG. 42 depicts a N-6-fluorescein-N′-chlorambucil-Lys-OH;

FIG. 43 depicts N-Tris(BG-PEG4-NH-carbonylethyloxymethyl)-methyl N′-5-fluorescein-N″-chlorambucil-Lys-amide;

FIG. 44 depicts a 6-fluorescein derivative corresponding to the embodiment of FIG. 43;

FIG. 45 depicts N-5-Fluorescein-N′-6-maleimidohexanoyl-Lys-OH;

FIG. 46 depicts N-6-fluorescein-N′-6-maleimidohexanoyl-Lys-OH;

FIG. 47 depicts N-Tris(BG-PEG4-NH-carbonylethyloxymethyl)methyl N′-5-fluorescein-N″-6-maleimidohexanoyl-Lys-amide;

FIG. 48 depicts a 6-fluorescein derivative corresponding to the embodiment of FIG. 47;

FIG. 49 depicts N-Tris(BG-PEG4-NH-carbonylethyloxymethyl)methyl N′-5-fluorescein-N″-6-maleimidohexanoyl-Lys-amide siRNA conjugate;

FIG. 50 depicts a 6-fluorescein derivative corresponding to the embodiment of FIG. 49;

FIG. 51 depicts a N-Tris(BG-PEG4-NH-carbonylethyloxymethyl)methyl N′-2-(2-(2-(2-(N″-5-fluorescein-N′″-chlorambucil-Lys-amido)ethoxy)ethoxy)ethoxy)-ethyl-urea;

FIG. 52 depicts a 6-fluorescein derivative corresponding to the embodiment of FIG. 51;

FIG. 53 depicts N-Tris(BG-PEG4-NH-carbonylethyloxymethyl)methyl N′-2-(2-(2-(2-(N″-5-fluorescein-N′″-6-maleimidohexanoyl-Lys-amido)ethoxy)ethoxy)ethoxy)ethyl-urea;

FIG. 54 depicts a 6-fluorescein derivative corresponding to the embodiment of FIG. 53;

FIG. 55 depicts N-Tris(BG-PEG4-NH-carbonylethyloxymethyl)methyl N′-2-(2-(2-(2-(N″-5-fluorescein-N′″-6-maleimidohexanoyl-Lys-amido)ethoxy)ethoxy)-ethoxy) [symbol] ethyl-urea siRNA conjugate;

FIG. 56 depicts a 6-fluorescein derivative corresponding to the embodiment of FIG. 55;

FIG. 57 depicts N-Tris-{2-(2-(2-(CoA-S-succinimido)ethylaminocarbonyl-PEG12)ethyl [symbol] aminocarbonyl)ethoxymethyl}-methyl N′-5-fluorescein-N″-chlorambucil-Lys-amide;

FIG. 58 depicts a 6-fluorescein derivative corresponding to the embodiment of FIG. 57;

FIG. 59 depicts N-Tris-{2-(2-(2-(CoA-S-succinimido)ethylaminocarbonyl-PEG12)ethyl [symbol] aminocarbonyl)ethoxymethyl}methyl N′-5-fluorescein-N″-6-maleimido [symbol] hexanoyl-Lys-amide;

FIG. 60 depicts a 6-fluorescein derivative corresponding to the embodiment of FIG. 59;

FIG. 61 depicts N-Tris-{2-(2-(2-(CoA-S-succinimido)ethylaminocarbonyl-PEG12)ethyl [symbol] aminocarbonyl)ethoxymethyl}methyl N′-5-fluorescein-N″-6-maleimido [symbol] hexanoyl-Lys-amide siRNA conjugate;

FIG. 62 depicts a 6-fluorescein derivative corresponding to the embodiment of FIG. 61;

FIG. 63 depicts N-Tris{2-(2-(2-(CoA-S-succinimido)ethylaminocarbonyl-PEG12)ethyl [symbol] aminocarbonyl)ethoxymethyl}methyl N′-2-(2-(2-(2-(N″-5-fluorescein-N′″-chlorambucil-Lys-amido)ethoxy)ethoxy)ethoxy)ethyl-urea;

FIG. 64 depicts a 6-fluorescein derivative corresponding to the embodiment of FIG. 63;

FIG. 65 depicts N-Tris{2-(2-(2-(CoA-S-succinimido)ethylaminocarbonyl-PEG12)ethyl [symbol] aminocarbonyl)ethoxymethyl}methyl N′-2-(2-(2-(2-(N″-5-fluorescein-N′″-6-maleimidohexanoyl-Lys-amido)ethoxy)ethoxy)ethoxy)ethyl-urea;

FIG. 66 depicts a 6-fluorescein derivative corresponding to the embodiment of FIG. 66;

FIG. 67 depicts N-Tris{2-(2-(2-(CoA-S-succinimido)ethylaminocarbonyl-PEG12)ethyl [symbol] aminocarbonyl)ethoxymethyl}methyl N′-2-(2-(2-(2-(N″-5-fluorescein-N′″-6-maleimidohexanoyl-Lys-amido)ethoxy)ethoxy)ethoxy)ethyl-urea siRNA conjugate;

FIG. 68 depicts a 6-fluorescein derivative corresponding to the embodiment of FIG. 67;

FIG. 69 depicts 2-Phthalimido-N-(BG-PEG4)-succinic acid monoamide;

FIG. 70 depicts N-4-((4-Aminopyrimidin-2-yloxy)methyl)benzyl-N′-2-(2-(2-(2-azidoethoxy) [symbol] ethoxy)ethoxy)ethyl-urea;

FIG. 71 depicts N-4-((4-Aminopyrimidin-2-yloxy)methyl)benzyl-N′-2-(2-(2-(2-aminoethoxy) [symbol] ethoxy)ethoxy)ethyl-urea;

FIG. 72 depicts N-4-((4-Aminopyrimidin-2-yloxy)methyl)benzyl-N′-2-(2-(2-(2-(3-BG-PEG4-NH-carbonyl-2-phthalimido-propionylaminoethoxy)ethoxy)-ethoxy)ethyl-urea;

FIG. 73 depicts N-4-((4-Aminopyrimidin-2-yloxy)methyl)benzyl-N′-2-(2-(2-(2-(3-BG-PEG4-NH-carbonyl-2-amino-propionylaminoethoxy)ethoxy)ethoxy)ethyl-urea;

FIG. 74 depicts N-4-((4-Aminopyrimidin-2-yloxy)methyl)benzyl-N′-2-(2-(2-(2-(3-BG-PEG4-NH-carbonyl-2-(fluorescein-5-carboxamido)propionylamino-ethoxy) [symbol] ethoxy)ethoxy)ethyl-urea;

FIG. 75 depicts a fluorescein-6-carboxamide corresponding to the embodiment of FIG. 74;

FIG. 76 depicts N-4-((4-Aminopyrimidin-2-yloxy)methyl)benzyl-N′-2-(2-(2-(2-(3-BG-PEG4-NH-carbonyl-2-(6-maleimidohexanoylamino)propionyl-amino-ethoxy)ethoxy) [symbol] ethoxy)ethyl-urea;

FIG. 77 depicts N-4-((4-Aminopyrimidin-2-yloxy)methyl)benzyl-N′-2-(2-(2-(2-(3-BG-PEG4-NH-carbonyl-2-(6-maleimidohexanoylamino)propionylamino-ethoxy)ethoxy) [symbol] ethoxy)ethyl-urea siRNA conjugate;

FIG. 78 depicts N-4-((4-Aminopyrimidin-2-yloxy)methyl)benzyl-N′-2-(2-(2-(2-(3-BG-PEG4-NH-carbonyl-2-chlorambucilcarboxamino-propionylaminoethoxy)ethoxy) [symbol] ethoxy)ethyl-urea;

FIG. 79 depicts BG-PEG12-NH Fmoc;

FIG. 80 depicts BG-PEG12-NH₂;

FIG. 81 depicts Tris{[2-carboxyethoxy]methyl}methylamine;

FIG. 82 depicts N-Tris[(2-carboxyethoxy)methyl]methyl 7-(diethylamino)coumarin-3-carboxamide;

FIG. 83 depicts N-Tris{[2-(tertbutoxycarbonyl)ethoxy]methyl}methyl ATTO-495-carboxamide;

FIG. 84 depicts N-Tris[(2-carboxyethoxy)methyl]methyl ATTO-495-carboxamide;

FIG. 85 depicts N-Tris{[2-(tert-butoxycarbonyl)ethoxy]methyl}methyl nile red-oxyacetamide;

FIG. 86 depicts N-Tris[(2-carboxyethoxy)methyl]methyl nile red-oxyacetamide;

FIG. 87 depicts N-Tris{[2-(tert-butoxycarbonyl)ethoxy]methyl}methyl 5-maleimidopentanecarboxamide;

FIG. 88 depicts N-Tris[(2-carboxyethoxy)methyl]methyl 5-maleimido-pentanecarboxamide;

FIG. 89 depicts N-Tris-{[2-(BG-PEG12-NH)-carbonylethoxy]methyl}methyl 7-(diethylamino) [symbol] coumarin-3-carboxamide;

FIG. 90 depicts N-Tris-{[2-(BG-PEG12-NH)-carbonylethoxy]methyl}-methyl ATTO-495-carboxamide;

FIG. 91 depicts N-Tris-{[2-(BG-PEG12-NH)-carbonylethoxy]methyl}-methyl nile red-oxyacetamide;

FIG. 92 depicts N-Tris-{[2-(BG-PEG12-NH)-carbonylethoxy]methyl}-methyl 5-maleimido [symbol] pentanecarboxamide;

FIG. 93 depicts 3-[2-(2-maleimidoethyl)disulfanyl]propanoic acid;

FIG. 94 depicts N-Tris-{[2-(tert-butoxycarbonyl)ethoxy]methyl}methyl 3-[2-(2-maleimido [symbol] ethyl)disulfanyl]propanoylamide;

FIG. 95 depicts N-Tris[(2-carboxyethoxy)methyl]methyl 3-[2-(2-male-imidoethyl)disulfanyl] [symbol] propanoylamide;

FIG. 96 depicts N-Tris-{[2-(BG-PEG12-NH)-carbonylethoxy]methyl}-methyl-3-[2-(2-maleimidoethyl)disulfanyl]propanoylamide;

FIG. 97A depicts a vector;

FIG. 97B depicts an alternative embodiment of a vector;

FIG. 97C depicts an alternative embodiment of a vector;

FIG. 97D depicts alternative embodiments of a vector;

FIG. 97E depicts an alternative embodiment of a vector;

FIG. 97F depicts an alternative embodiment of a vector;

FIG. 97G depicts an alternative embodiment of a vector;

FIG. 98a exemplifies an embodiment of an open reeading frame;

FIG. 98b exemplifies an embodiment of an open reeading frame;

FIG. 98c exemplifies an embodiment of an open reeading frame;

FIG. 98d exemplifies an embodiment of an open reeading frame;

FIG. 98e exemplifies an embodiment of an open reeading frame;

FIG. 98f exemplifies an embodiment of an open reeading frame;

FIG. 98g exemplifies an embodiment of an open reeading frame;

FIG. 98h exemplifies an embodiment of an open reeading frame;

FIG. 98i exemplifies an embodiment of an open reeading frame;

FIG. 98j exemplifies an embodiment of an open reeading frame;

FIG. 99a depicts the result of a SDS-PAGE and Coomassie staining test;

FIG. 99b depicts an embodiment of a crosslinker;

FIG. 99c depicts a result of a labeling reaction;

FIG. 99d depicts a result of a labeling reaction;

FIG. 100 depicts a composite picture of Hai-SNAP fusion protein labeled with three different fluorophor labeled homotrimeric crosslinkers and visualized by an in vivo imager, (a) composite picture, (b) stained gel, (c) the gel analyzed densiometricly, (d) a confocal microscopy done with the SV305 crosslinked version of Hai-SNAP;

FIG. 101 shows a 12% SDS-PAGE gel (A: UV light, B: Coomassie stained) which was loaded with 5 μg of the different mammalian expressed and IMAC (Immobilized Metal Affinity Chromatography) purified. The Gel contains: 1:Ki4-SNAP; 2: SNAP-EGF; 3: Hai-SNAP; 4: H22-SNAP; 5: SNAP-CD30L; M: prestained protein marker (NEB);

FIG. 102A shows a confocal microscopy of Ki4-SNAP functionalized Nanobeads binding CD30-positive L540 cells. Rhodamine based emission of beads in red (A) and Draq5 emission in blue pseudocolour (B), overlay (D) with grayscale picture (C).

FIG. 102B shows the flow cytometric analysis of cD30 overexpressing L540cy cells incubated with different amounts (0.5 and 5 μl) of Ki4-SNAP coupled rhodamine doted nanobeads. As control 5 μl uncoupled beads were applied to L540cy cells.

FIG. 102C shows the flow cytometric analysis of the CD30 negative U937 cells incubated with different amounts (0.5 and 5 μl) of Ki4-SNAP coupled rhodamine doted nanobeads. As control 5 μl uncoupled beads were applied to the U937 cells.

FIG. 103 depicts a schematic overview of a sandwich ELISA protocol for an embodiment of the invention;

FIG. 104A shows a schematic procedure for si-RNA coupling to SNAP-tag fusion proteins;

FIG. 104B is an ethidiumbromide stained gel analyzed under a standard UV transilluminator;

FIG. 104C is the same ge as in FIG. 104Bl subsequently Coomassie stained;

FIG. 105A shows an evaluation of flow cytometric analysis of Ki-SNAP labeled with SNAP-vista Green (Covalys) binding on the CD30 overexpressing cell line L540cy;

FIG. 105B shows an evaluation of flow cytometric analysis of SNAP-CD30L labeled with SNAP-vista Green (Covalys) binding on the CD30 overexpressing cell line L540cy;

FIG. 105C shows an evaluation of flow cytometric analysis of Hai-SNAP labeled with SNAP-vista Green (Covalys) binding on the EGFR overexpressing cell line A431;

FIG. 105D shows an evaluation of flow cytometric analysis of SNAP-EGF labeled with SNAP-vista Green (Covalys) binding on the EGFR overexpressing cell line A431;

FIG. 105E shows an evaluation of flow cytometric analysis of H22-SNAP labeled with SNAP-vista Green (Covalys) binding on the CD64 overexpressing cell line U937 and not binding on the CD64 negative cell line L540;

FIG. 105F depicts a negative control for the embodiment of FIG. 105E;

FIG. 106 shows confocal pictures of L540cy cells stained with BG505 (Covalys) labeled Ki4-SNAP;

FIG. 107 shows infrared pictures from the whole mouse that were taken and analyzed by spectral unmixing the signal from background with the Intas Cri-Maestro In vivo imager;

FIG. 108 shows infrared pictures from the whole mouse that were taken and analyzed by spectral unmixing the signal from background with the Intas Cri-Maestro In vivo imager;

FIG. 109 shows confocal pictures of L3.6pl cells stained with BG505 (Covalys) labeled Hai-SNAP. There is a clear higher internalization rate of bound Hai-SNAP into the cells when incubated at 37° in comparison to the 4° C. sample; Panel A shows A shows internalized HaiSNAP-BG505; Panel B shows clathrin-mediated internalization of transferrin-ALEXA594, Panel C shows an overlay of A and B; Panel D is an overlay of C with transmission light picture; Panel E is a magnification of Panel D: arrows depict vesicles harboring both labeled transferrin and HaiSNAP-BG505. There is a high degree of overlapping localization of transferrin and HaiSNAP-BG505;

FIG. 110 shows the colocalization of Hai-SNAP BG505 labeled and transferrin ALEXA 594 labeled after internalization;

FIG. 111 shows the TMR staining of HEK293 cells expressing the Hai-SNAP fusion protein together with the EGFP reporter protein which is encoded 3′ on the biscistronic mRNA. Panel A shows the signal of the EGFP reporter, Panel B shows the TMR signal belonging to the SNAP-Tag fusion protein, Panel C shows the Draq5 nuclear counterstain and Panel D is a transmission light picture of the same cells;

### DETAILED DESCRIPTION OF THE INVENTION

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

The invention also relates to diagnostic, therapeutic or analytical compositions of the heterologous complex, methods of producing such complexes and methods of using the same in vitro and in vivo.

As used herein, the specification, “a” or “an” may mean one ore more. As used herein in the claim(s), when used in conjunction with the word “comprising”, the words “a” and “an” may mean one or more than one. As used herein “another” may mean at least a second or more.

As used herein, the term “component A” of the complex represents the actively binding structure of the complex of present invention. The component A is selected from the group of actively binding structures consisting of antibodies or their derivatives or fragments thereof, synthetic peptides such as scFv, mimotopes, etc. or chemical molecules such as carbohydrates, lipids, nucleic acids, peptides, vitamins, etc., and/or small molecules with up to 100 atoms with receptor-binding activity like ligands, in particular single atoms, peptidic molecules, non-peptidic molecules, etc., and/or cell surface carbohydrate binding proteins and their ligands such as lectins, in particular calnexins, c-type lectins, l-type lectins, m-type lectins, p-type lectins, r-type lectins, galectins and their derivatives, and/or receptor binding molecules such as natural ligands to the cluster of differentiation (CD) antigens, like CD30, CD40, etc., cytokines such as chemokines, colony stimulating factors, type-1 cytokines, type-2 cytokines, interferons, interleukins, lymphokines, monokines, etc., and/or adhesion molecules including their derivatives and mutants, and/or derivatives or combinations of any of the above listed of actively binding structures, which bind to CD antigens, cytokine receptors, hormone receptors, growth factor receptors, ion pumps, channel-forming proteins. The component A may also be selected from the group of passively binding structures consisting of allergens, peptidic allergens, recombinant allergens, allergen-idiotypical antibodies, autoimmune-provoking structures, tissue-rejection-inducing structures, immunoglobulin constant regions and their derivatives, mutants or combinations thereof. Combining at least two identical or different binding structures selected from the above-mentioned groups may generate a component A with higher valency.

In an additional object of the present invention, component A is binding to a cell surface marker of a healthy or diseased cell belonging to the cluster of differentiation antigens (CD-antigens, Table 1).

In another specific embodiment, the component A is a chemokine or a specifically binding fragment thereof like those provided in table 2 binding to its specific cellular receptors.

In another embodiment, component A is an interleukin or a specifically binding fragment thereof like those provided in table 3 binding to its specific cellular receptor.

In another embodiment, component A is the extracellular or intracellular part of a cluster of differentiation antigens as listed in table 1 specifically binding to soluble factors and being used to detect a soluble antigen or a family of soluble antigens.

In another specific embodiment, the component A is an angiogenic factor modulating growth, chemotactic behavior and/or functional activities of vascular endothelial cells or a specifically-binding fragment thereof including AcSDKP, aFGF, ANF, Angiogenin, angiomodulin, Angiotropin, AtT20-ECGF, B61, bFGF, bFGF inducing activity, CAM-RF, ChDI, CLAF, ECGF, ECI, EDMF, EGF, EMAP-2, Neurothelin (see: EMMPRIN), Endostatin, Endothelial cell growth inhibitor, Endothelial cell-viability maintaining factor, Epo, FGF-5, IGF-2 (see: Growth-promoting activity for vascular endothelial cells), HBNF, HGF, HUAF, IFN-gamma, IL1, K-FGF, LIF, MD-ECI, MECIF, NPY, Oncostatin M, PD-ECGF, PDGF, PF4, PIGF, Prolactin, TNF-alpha, TNF-beta, Transferrin, VEGF. Some of these factors are protein factors detected initially due to some other biological activities and later shown to promote angiogenesis. The list of protein factors angiogenically active in vivo includes fibroblast growth factors (see: FGF), Angiogenin, Angiopoietin-1, EGF, HGF, NPY, VEGF, TNF-alpha, TGF-beta, PD-ECGF, PDGF, IGF, IL8, Growth hormone. Fibrin fragment E has been shown also to have angiogenic activity. In addition there are factors such as Angiopoietin-1, which do not behave as classical growth factors for endothelial cells but play a prominent role in vasculogenic and angiogenic processes.

In another embodiment, the component A is a virulence factor or the corresponding part of it binding to a subset of human cells such as 121R, 14.7 kDa orf virus protein, 145R, 16 kDa orf virus protein, 2C, 38K gene of Cowpox virus, 3a, 5EL, 5-HL, 7a, A224L, A238L, A39R, A41L, AcMNPV ORF32, Actinobacillus actinomycetem comitans Cytolethal distending toxin, Actinobacillus actinomycetem comitans leukotoxin, Adenovirus Death Protein, Adenovirus E1B 19 kDa protein, Adenovirus E3 10.4K/14.5 kDa protein, Adenovirus E3 14.7 kDa protein, Adenovirus E3 19 kDa protein, Aerolysin, AgMNPV IAP3, AHV-Sema, AIP56, Alpha-Hemolysin, alpha-HL, Alpha-toxin, Anti-cytokines, Apoptin, Apoptosis, B13R, B15R, B18R, B8R, Bacillus anthracis toxin, Bacteriokine, baculovirus p35 protein, baculovirus P49 protein, BAD1, BALF-1, BARF1, BCK, BCL2, BCRF-1, Beta-Hemolysin, Beta-toxin, BHRF-1, Bm-MIF, BmNPV FGF, Bordetella dermonecrotic toxin, BORFE2, BPV-1 E6, BZLF1, C12L, C21L, CADD, Campylobacter Cytolethal distending toxin, caspase-7-like protein, Caspases, CDT, Ce-MIF, Chemokines, Circovirus type 2 ORF3, CLAP, Clostridium perfringens alpha-toxin, Clostridium perfringens beta-toxin, CMV IL10, CMV RR1, CNF1, CNF2, COPE version 15.8, COPE version 8.7, COPE, crmA, crmB, crmC, crmD, crmE, Cytokine assays, Cytokine Inter-species Reactivities, Cytokines, D7L, Delta-hemolysin, Delta-toxin, E1.1, E1B-55K, E2, E3-6.7, E3L, E3L-like protein, E4orf4, E5, E6, E7, E8, Early response gene, EBNA-LP, ECRF-3, ectromelia poxvirus p13, Ectromelia virus p28 protein, EHV-2 E1 ORF, EHV-2 IL10, EP153R, EP402R, Erns, Escherichia coli Cytolethal distending toxin, F1L, FLIP, FPV016 protein, Fractalkine, Fumonisins, Fusobacterium necrophorum leukotoxin, G4R, G5R, GAM-1, Gamma-hemolysin, GIF, glycoprotein G, gp120, GPCMV-MIP, H3L, H4R, H83, Haemophilus ducreyi Cytolethal distending toxin, HBx, Helicobacter Cytolethal distending toxin, hemolysin BL, Herpesvirus saimiri BCL2, HJ1, HP1118, HP-NAP, HSGF-2, HVP IL10, HVS13, IAP, ICP0, ICP10PK, ICP22, ICP27, ICP34.5, IE1, IE2, IE2579aa, IMP, Influenza A virus NS1 protein, IpaB, ITA, K13, K2, K2R, K3R, K4.1, K6, KSHV ORF4, KSHV, L*, LANA-2, Leishmania mexicana cysteine protease CPB2.8, LMP2A, M11L, m131/129, M3, M33, M78, MALP-404, Mannheimia haemolytica leukotoxin, MC148R, MC159, MC53L, MC54L, MDM, MDV003, MDV078, MEQ, MGF, Microcystin-LR, Microkine, Modulins, M-T1, M-T7, MyD, N1R, Nipah virus P protein, Nipah virus V protein, Nipah virus W protein, Npro, NS1, NS2, NS5A, orf virus IL10, orf virus, ORF, ORF13, ORF152, ORF16, ORF390, ORF45, ORF50, ORF74, ORFK2, ORFK4.1, ORFK4, ORFK5, ORFK6, ORFK7, ORFK9, ORFV2-VEGF, p13, Panton-Valentine leukocidin, Pasteurella multocida toxin, PB1-F2, Poxvirus growth factor, PRGF, Pseudomonas aeruginosa exotoxin A, RK-BARF0, RRV ORF74, RSV Glycoprotein G, RTA, SARS coronavirus E protein, SARS coronavirus N protein, SARS coronavirus non-structural protein-1, SCMV IL10, SERP1, SERP2, SERP3, SFGF, Shigella Cytolethal distending toxin, sigmaC, SipB, sis, Sliap, Slp49, SPI-2, SPV146, Staphylococcus aureus alpha-toxin, Staphylococcus aureus delta-toxin, Staphylococcus aureus gamma-toxin, STI, Streptolysin O, SV40 large T antigen, SV5 V protein, swinepox virus SPV003/148 protein, T2, TAIP, Tanapoxvirus 2L protein, Tanapoxvirus 38 kDa protein, Thogoto virus ML protein, Trypanokine, U12, U51, U83, U83A, UL111.5A, UL111a, UL119-UL118, UL141, UL144, UL146, UL147, UL18, UL3 protein, UL36, UL37, UL69 protein, UL82, US27, US28, US3, Us5, V protein, VacA, Vaccinia 19 kDa protein, Vaccinia growth factor, Vaccinia virus growth factor, Vaccinia virus protein phosphatase VH1, vBCK, vBCL2, vC4bBP, vCCI, vCCL1, vCKBP, vCKBP-1, vCKBP-2, vCKBP-3, vCKBP-4, VCP, vCSF1BP, VEGF-E, vFGF, VG71, vGPCR, vICA, vIL17, vIL18BP, vIL6, vIL8, viral BCK, viral BCL2, viral C4b binding protein, viral CCL1, viral CD30, viral chemokine binding protein, viral chemokine binding protein-i, viral chemokine binding protein-2, viral chemokine binding protein-3, viral chemokine binding protein-4, viral chemokine inhibitor, viral CSF1 binding protein, viral cytokine receptors, viral cytokines, viral EGF, viral Fc-gamma R2, viral Fc-gamma R3, Viral FLICE-inhibitory proteins, viral G-protein-coupled receptor, viral IFN-gamma/IL2/IL5 binding protein, viral IL10, viral IL17, viral IL18 binding protein, viral IL6, viral IL8, viral inhibitor of apoptosis protein, viral inhibitor of caspase activation, viral interferon regulatory factor, viral interferon regulatory factor-1, viral interferon regulatory factor-2, viral interferon regulatory factor-3, viral M-CSF binding protein, viral MIP-1, viral MIP-1-alpha, viral MIP-1-beta, viral MIP-2, viral NGF-beta, viral OX2, viral semaphorin, viral TGF-beta, viral VEGF, vIRF, vIRF1, vIRF2, vIRF3, Viroceptor, Virokine, vMCC-1, vM-CSFBP, vMIA, vMIP-1, vMIP-1-alpha, vMIP-1-beta, vMIP-2, vMIP-3, vNGF-beta, vOX2, VP35 protein, VP5, vTGF-beta, vTNFR, VVGF, Y134R, Yaba monkey tumor virus 2L protein, YLDV IL10, YopJ, ZmpB, Zta.

As used herein, the term “antibody” refers to polyclonal antibodies, monoclonal antibodies, humanized antibodies, single-chain antibodies, and fragments thereof such as Fab, F(ab′)2, Fv, and other fragments which retain the antigen binding function and specificity of the parent antibody.

As used herein, the term “monoclonal antibody” refers to an antibody composition having a homogeneous antibody population. The term is not limited regarding the species or source of the antibody, nor is it intended to be limited by the manner in which it is made. The term encompasses whole immunoglobulins as well as fragments such as Fab, F(ab′)2, Fv, and others which retain the antigen binding function and specificity of the antibody. Monoclonal antibodies of any mammalian species can be used in this invention. In practice, however, the antibodies will typically be of rat or murine origin because of the availability of rat or murine cell lines for use in making the required hybrid cell lines or hybridomas to produce monoclonal antibodies.

As used herein, the term “human antibodies” means that the framework regions of an immunoglobulin are derived from human immunoglobulin sequences.

As used herein, the term “single chain antibody fragments” (scFv) refers to antibodies prepared by determining the binding domains (both heavy and light chains) of a binding antibody, and supplying a linking moiety, which permits preservation of the binding function. This forms, in essence, a radically abbreviated antibody, having only that part of the variable domain necessary for binding to the antigen. Determination and construction of single chain antibodies are described in U.S. Pat. No. 4,946,778 to Ladner et al.

The component B is an enzyme like protein derived from the Alklguanine-DNA-alkyltransferase (AGT), which has a substrate specificity for O⁶-benzylguanine or O⁶ heteroarylmethylguanine. The enzyme like protein is able to transfer a certain label from the substrate in a reaction previously described in WO/2005/085470.

In a specific embodiment the enzyme like protein has been modified to recognize 2-amino-4-benzyloxypyrimidines as described in WO/2006/114409.

The component B may also be an enzyme like protein derived from the protein Alkylcytosine transferase (ACT), which has the substrate specificity for O²-benzylcytosine derivatives and related O² heteroarylmethyl-cytosine derivatives described previously in WO/2008/012296.

In an alternate embodiment of the invention B consists of an Acyl carrier protein or fragments thereof. Coenzym A derivatives are able to transfer their label to the ACP or part of the ACP in the presence of the modifying enzyme holo-acyl carrier protein (ACPS) or modification or mutants thereof as previously described in WO/2004/104588.

The DNA sequences of the invention may be engineered in order to alter a chimeric coding sequence for a variety of modifications, including but not limited to alterations, which modify processing, and expression of the gene product. For example, mutations may be introduced by techniques which are well known in the art, for example site directed mutagenesis or SOE-PCR to insert or remove restriction sites, to alter glycosylation or phosphorylation pattern or to alter the substrate specificity of the active center.

As used herein, the term “component C” of the complex represents a specific additional function added to the complex AB through covalent coupling. Component C is a drug, a detectable label or other components mediating biological activity in a targeted cell or organism. C can also be a solid phase.

C further contains a moiety which serves as a substrate for component B.

Component C can have the structure (X)ₙ₁-(Y)-(Z)ₙ₂ with X being a component B specific substrate and n1 being one or more preferentially 1-3 and Z being a drug, a detectable label or other components mediating biological activity in a targeted cell or organism and n2 being 1 or more.

Y is a linker structure designed to functionally connect X and Z. Y may fullfill the following functions: a spacer mediating the desired flexibility between X and Z (and this way between B and C) ensuring the functionality of each component within the assembled complex.

Further the linker may contain structures enabling the controlled release of Z under certain environmental conditions (e.g. pH sensitive or reducable structures for release in endosomes or the cytosol or enzyme degradable linkers). Such linker structures may be e.g. cis-Aconityl linkages, linkers containing an ester bond, acid sensitive hydrazone linkers, lysosomally degradable peptide linkers, self eliminating spacers, sulphhydryl linkers, light sensitive linkers (reviewed in Dyba et al.)

Further the linker may contain chelating agents such as DOTA (1,4,7,10-tetraazacyclododecane-1,4,7,10-tetraacetic acid) or DTAP (Diethylene triamine pentaacetic acid) that can be used for complexing e.g. radioisotopes.

Y may have linear, branched, tree like or polymeric structure.

Drugs considered as component C include all kinds of substances that can display their mode of action on the targeted cell and that are likely to be more effective when transported to a particular site within the body. Preferentially these are compounds with proven efficacy e.g. as chemotherapeutical agents. They may be selected from the group of alkylating agents (e.g. cyclophosphamide, chlorambucil), anthracyclins (doxorubicin, daunomycin), maytansinoids (maytansinoid DM1), anti-metabolites, plant alkaloids and terpenoids as the Vinca alkaloids (vinblastine, vincristine vinorebline, vindesin) Podophyllotoxin and derivatives hereof and taxanes (paclitaxel, docetaxel, taxotere) or topoisomerase inhibitors (camptothecins), synthetic toxins as ellipticine analogs or synthetic analogs of tumor antibiotics as duocarmycin or CC1065, other tubulin binding agents as halichondrin B, hemiasterlins and dolastatins or analogs as monomethyl-auristatin E; component C may also be selected from the group of small molecules having cytotoxic/cytostatic activities like alkylating agents (like Cyclophosphamide, Mechlorethamine, Chlorambucil, Melphalan) or anthracyclines (like Danorubicin, Doxorubicin, Epirubicin, Idarubicin, Mitoxantrone, Valrubicin) or cytoskeletal disruptors (like Paclitaxel, Docetaxel) or Epothilones (like) or Inhibitors of topoisomerase II (like Etoposide, Teniposide, Tafluposide) or nucleotide analogs and precursor analogs (like azacididine, azathioprine, capecitabine, cytarabine, doxofluridine, fluorouracil, gemcitabine, mercaptopurine, methotrexate, tioguanine) or peptide antibiotics (like bleomycin) or platinum-based agents (like carboplatin, cisplatin, oxaliplatin) or retinoids (like all-trans retinoic acid) or vinca alkaloids and derivatives (like vinblastine, vincristine, vindestine, vinorelbine), beta ray emiting nuclides like Iodine-131, Yttrium-90, Lutetium-177, from the group of Aromatase Inhibitors (like Aminoglutethimide, Anastrozole, Letrozole, Vorozole, Exemestane, 4-androstene-3,6,17-trione, 1,4,6-androstatrien-3,17-dione, Formestane, Testolactone), Carbonic Anhydrase Inhibitors (like Acetazolamide, Methazolamide, Dorzolamide, Topiramate), Cholinesterase Inhibitors (Organophosphates like Metrifonate, Carbamates like Physostigmine, Neostigmine, Pyridostigmine, Ambenonium, Demarcarium, Rivastigmine, Phananthrine like Galantamine, Piperidine like Donepezil, Tacrine, Edophonium, or Phenothiazines), Cyclooxygenase Inhibitors (like Celecoxib, Rofecoxib, Etoricoxib, Acetaminophen, Diclofenac, Ibuprofen), Folic Acid Antagonists (like Methotrexate), Hydroxymethylglutaryl-CoA Reductase Inhibitors (like Atorvastatin, Cerivastatin, Fluvastatin, Lovastatin, Mevastatin, Pitavastatin, Pravastatin, Rosuvastatin, Simvastatin, Vytorin, Advicor, Caduet), Integrase Inhibitors (like Raltegravir, Elvitegravir), Lipoxygenase Inhibitors (like Zileutron), Monoamine Oxidase Inhibitors (like Isocarboxazid, Moclobemide, Phenelzine, Tranylcypromine, Selegiline, Rasagiline, Nialamide, Iproniazid, Iproclozide, Toloxatone, Linezolid, Tryptamines, Dienolide, Detxtroamphetamine), Nucleic Acid Synthesis Inhibitors, Phosphodiesterase Inhibitors (like Caffeine, Theopyline, 3-isobutyl-1-methylxanthine, Vinpocetine, EHNA, Enoximone, Lirinone, PDE3, Mesembrine, Rolipram, Ibudilast, Sildenafil, Tadalafil, Vardenafil, Udenafil, Avanafil), Protease Inhibitors (like Saquinavir, Ritonavir, Idinavir, Nelfinavir, Amprenavir, Lopinavir, Atazanavir, Fosamprenavir, Tipranavir, Darunavir), Protein Kinase Inhibitors (like Imatinib, Geftinib, Pegaptanib, Sorafenib, Dasatinib, Sunitinib, Erlotinib, Nilotinib, Lapatinib), Protein Synthesis Inhibitors (like Anisomycin, Cycloheximide, Chloramphenicol, Tetracycline, Streptomycin, Erythromycin, Puromycin, etc.), Proton Pump Inhibitors (like Omeprazole, Lansoprazole, Esomeprazole, Pantoprazole, Rabeprazole), from the group of oligonucleotides nucleic acids like small interfering RNAs (siRNAs) or a short hairpin RNA (shRNA), an antisense DNA or RNA, a double stranded RNA (dsRNA) or a micro RNA (miRNA) might be used to down-regulate specific key elements of regulative pathways within a cell.

In a specific embodiment component C is a polymer or dendrimer carrying several cytostatic/cytotoxic agents as exemplified above like e.g. paclitaxel or methothrexat molecules carrying a Benzylguanine (BG)/Benzylcytosine(BC)-group and is modified to improve biocompatibility e.g. by pegylation.

Further the drug can be a radioisotope selected from the group of beta emiting isotopes that can be used for radiotherapy (e.g. iodine-131, lutetium-177, yttrium 90).

In another example the drug can be a nucleic acid or a nucleic acid analog, which can exert biological activity in the targeted cell. More specifically the nucleic acid molecule can be designed to allow the expression of an encoded protein in the targeted cell (in the sense of a gene therapy) or to mediate RNA interference (RNAi) including small interfering RNAs (siRNAs) or a short hairpin RNA (shRNA), an antisense DNA or RNA, a double stranded RNA (dsRNA) or a micro RNA (miRNA).

In a specific embodiment component C is a siRNA or a linker structure as defined hereinbefore with one or more functionally attached siRNAs of a single specificity or several different specificities. The RNAi mediating compound may be directed against any desired cellular mRNA. The RNA interference (RNAi) mediating compound may be designed to directly or indirectly downregulate the expression of factors that are essential for the survival of the targeted cell (e.g. siRNA mediated knock down of elongation factor II (eEFII or a variety of anti-apoptotic factors as BCL2, BCL-xL or other oncogenes) or may be designed to alter the gene expression profile in a targeted cell in a way that has a therapeutic effect.

In a concrete example the complex AB-C comprises an EGFR specific single chain antibody or human EGF fused to the SnapTag, to which a siRNA directed against the human elongation factor II, laminA/C, or GFP modified with BG is coupled.

Component C may further be a prodrug that is activated e.g. by cellular proteases upon entry into the target cell.

The drug may further be a peptide or polypeptide that has toxic activity in the targeted cell.

Examples are the ADP ribosylating enzymes pseudomonas exotoxin A, diphteria-, cholera-, pertussis- and botulinotoxin. The ribosome inactivating proteins diathin, saporin, bryodin, gelonin, ricin, abrin or restrictocin. ribonucleases (Phosphodiesterases) RNAse H, angiogenin, eosinophil-derived neurotoxin (EDN), eosinophilic cationic protein, onconase and bullfrog lektin. Additional proteins that can be represented by C include prodrug activating enzymes as caliceamicin, glucoseoxidase, carboxypeptidase, alkaline phosphatase, cytosindeaminase, beta-glycosidase, beta-glucoronidase, beta-lactamase, nitroreductase, thymidinkinase or purine-nucleoside phosphorylase. Further cathepsines, granzymes and combinations and possible variations of the afore mentioned protein families.

Preferred are validated toxins as ricin A, alpha sarcin (family of lectins), diphteriatoxin and pseudomonas exotoxin A. They have been subject of several clinical studies and their efficasy is well documented.

Component C may also represent toxic peptides as denfensines, anti-fungal peptides or e.g. several peptides isolated from lumpfish or sponges.

Detectable labels are fluorescent dyes such as fluorescein, rhodamine, courmarine, and cyanine and derivatives hereof. Preferred fluorophores emitt in the near infra red (NIR) range between 680 and 950 nm. This wavelength results in very low background fluorescence and excellent tissue penetration and is therefore ideally suited for fluorescence detection in vivo. In a specific embodiment a tumour specific antibody or other ligand in fusion with the Snap-tag is labeled with a BG derivative of a NIR dye. The labeled antibody or ligand serves as an imaging tool that can be used to visualize tumor growth and/or treatment in vivo.

In a concrete example a BG derivative of an NIR dye emitting at 782 nm was coupled to a single chain antibody fragment SNAP-tag fusion protein targeting EGFR. The resulting in vivo imaging probe was used to detect EGFR expression in a pancreatic carcinoma xenograft model. In other concrete examples several fluorophore coupled complexes AB were used for flow cytometry and confocal microscopy applications.

Further the detectable label can be gamma emitting radioisotopes as e.g. iodine-131, lutetium-177, yttrium 90 or any other diagnostically relevant isotope usually combined with a complexing agent as DOTA or DTAP.

Further the detectable label can be a quantum dot composed of heavy metals like CdSe or InGaP. Quantum dots are favourable optical imaging agents due to their high quantum yield and photostability. Another possibility for a fluorescent label represented by component C may be noble metal nanoclusters composed of a few (8-12) gold or silver atoms, or synthetic fluorophores captured in nanoparticles made from silicon dixode.

Further detectable labels are superparamagnetic iron oxid particles for MRI based molecular imaging.

Fluorescent proteins like GFP or dsRED or derivatives hereof can serve as detectable label coupled to the complexes AB. Fluorescent proteins today cover a wide range of the visible spectrum as well as the near infrared.

Further detectable labels can be enzymes like alkaline phosphatase, peroxidases and galactosidases that are commonly applied in a variety of immunoassays.

Component C can also be a solid phase in the sense of a bead, a biochip surface or an ELISA-plate.

As used herein the term “antigen” is describing any target structure being bound by any component A.

As used herein the term “complex” is a chemical entity which may be constructed from different chemical structures forming a chemical compound, the different chemical structures linked to each other by covalent and/or ionic bonds, as well as hydrophobic and/or hydrophilic interactions.

As used herein the term “therapeutic” represents any use of the complex ABC that leads to at least stabilization of diseases.

As used herein the term “diagnostic” represents any use of the complex ABC which leads to the identification of the nature of problem in medicine, science, engineering, environment, food & feed, business, trade.

The term “target cell” and or “target tissue” refers to cells or tissues carrying an extracellular surface structure to which the component A of the complex actively or passively binds. Target cells and target tissues are thus cells and tissues to which the component A of the complex can bind.

The term “recombinant” refers to the preparation of molecules, in particular the covalent joining of molecules from different sources, by any one of the known methods of molecular biology. As used in the present invention, the term “recombinant” refers in particular to the fusion of the antibody or ligand part A to the enzyme like protein part B by any one of the known methods of molecular biology, such as through production of single chain antibodies. The recombinant DNA molecule encoding the recombinant fusion protein comprising the antibody/ligand part and the enzyme type protein part are recombinantly expressed. Recombinant invention related complexes produced in this way may be isolated by any technique known in the field of recombinant DNA expression technology suitable for this purpose.

The term “derivative” refers to a mutated or modified protein, which has retained its characterizing activity, i.e. binding activity or enzymatic activity. Particular preferred are constitutively active derivatives. The term derivative comprises proteins, which carry at least one amino acid substitution, deletion, addition, a swapping of a single domain or at least one modification of at least one amino acid. In particular derivatives having as many modifications as possible but not destroying the function of the compound of the invention are within the scope of the present invention more particularly those proteins which carry about 20 such changes or those with about 10 such changes or those with 1 to 5 such changes.

A further meaning of “derivative” is a chemical modification of a protein in its side chain, e.g. by glycosylation, phosphorylation, modification of carboxyl groups, such as amidation, esterification, modification of thiol or hydroxyl groups, e.g. by alkylation or oxidation or disulfide linking, modification of amino groups which may act as nucleophilic moiety, such as acylation, alkylation or other electrophilic attacks.

Further the term “derivative” refers to chemical structures analogous to a parent structure, which is extended or modified by another more or less complex group, e.g. a fluorophore being the parent structure extended by one or more reactive groups, e.g. a maleimido group.

As used herein, the term “As used herein, the term “vector” comprises DNA and RNA forms of a plasmid, a cosmid, a phage, phagemid, derivatives of them, or a virus. A vector comprises control sequences and coding sequences.

The term “expression of the recombinant genes encoding the recombinant complex”, wherein the recombinant complex is a single chain antibody/ligand-enzyme type protein fusion polypeptide, refers to the transformation and/or transfection of a host cell with a nucleic acid or vector encoding such a complex, and culturing said host cells selected from the group of bacteria, such as E. coli, and/or in yeast, such as in S. cerevisiae, and/or in established mammalian or insect cell lines, such as CHO, NS0, COS, BHK, 293T and MDCK cells, and/or in primary cells, such as human cells, non-human vertebrate cells, and/or in invertebrate cells such as insect cells, and the synthesis and translation of the corresponding mRNA, finally giving rise to the recombinant protein, the recombinant complex. In more detail, the term “expression of the recombinant genes encoding the recombinant complex”, comprises the following steps:

Transformation of an appropriate cellular host with a recombinant vector, in which a nucleotide sequence coding for the fusion protein had been inserted under the control of the appropriate regulatory elements, particularly a promoter recognized by the polymerases of the cellular host. In the case of a prokaryotic host, an appropriate ribosome-binding site (RBS) also precedes the nucleotide sequence coding for the fusion protein, enabling the translation in said cellular host. In the case of a eukaryotic host any artificial signal sequence or pre/pro sequence may be provided, or the natural signal sequence may be employed. The transformed cellular host is cultured under conditions enabling the expression of said insert.

Also claimed are cells or in vitro translation systems, which synthesize complete complexes according to the invention or individual components thereof, after transformation and/or transfection with, or addition of the nucleic acid molecules or vectors according to the invention.

One further embodiment of the present invention is a cellular compartment or an organism except a human being which compartment or organism being transformed or transfected with the nucleic acid according to the invention. The cellular compartment may be of prokaryotic origin in particular from E. coli, B. subtilis, S. carnosus S. coelicolor, and/or Marinococcus sp., or a lower eukaryote, such as Saccharomyces sp., Aspergillus sp., Hansenula polymorpha, Arxula adeninivorans, Spodoptera sp. and/or P. pastoris, a higher non-human eukaryote such as a plant and/or an animal, and the cell is a primary or cultivated mammalian cell, such as a freshly isolated human cell or a eukaryotic cell line such as CHO, NS0, COS, BHK, 293T and MDCK.

Cells or organisms according to the invention are either of prokaryotic origin, especially from E. coli, B. subtilis, S. carnosus, S. coelicolor, Marinococcus sp., or eukaryotic origin, especially from Saccharomyces sp., Aspergillus sp., Spodoptera sp., P. pastoris, primary or cultivated mammalian cells, eukaryotic cell lines (e.g., CHO, Cos or 293), plants (e.g. N. tabacum), or yeasts (e.g. S. cerevisiae, H. polymorpha, A. adenivorans).

The invention also relates to medicaments and analytical/diagnostic tools comprising the complex of the present invention and/or the nucleic acid or vectors encoding the complex of present invention. Typically, the complexes according to the invention are administered in physiologically acceptable dosage forms. These include, for example, Tris, NaCl, phosphate buffers and all approved buffer systems, especially including buffer systems, which are characterized by the addition of approved protein stabilizers. The administration is effected, in particular, by parenteral, intravenous, subcutaneous, intramuscular, intratumoral, transnasal administrations, and by transmucosal application.

The dosage of the complexes according to the invention to be administered must be established for each application in each disease to be newly treated by clinical phase I studies (dose-escalation studies).

The complex according to the invention, nucleic acid molecules coding therefore and/or cells or in vitro translation systems can be used for the preparation of a medicament for treating tumor diseases, allergies, autoimmune diseases, and chronic/acute inflammation reactions or for the preparation of a diagnostic tool for the same. Furthermore malignant diseases and tissue/graft rejection reactions can be treated.

Further details of recombinant protein engineering are either well known to the skilled person or become evident from Rosenblum in (US 2006/0280749 A1) incorporated herein by reference.

### EXAMPLES

The following is an illustration of preferred embodiments for practicing the present invention. However, they are not limiting examples. Other examples and methods are possible in practicing the present invention.

### I Chemical Synthesis of Component C

### ABBREVIATIONS

BC-NH2=2-(4-aminomethylbenzyloxy)-4-aminopyrimidine (aminomethylbenzylcytosine)
BG-PEG4-NH2=6-(4-((2-(2-(2-(2-aminoethoxy)ethoxy)ethoxy)ethoxy)methyl)benzyloxy)-9H-purin-2-amine (pegylated O⁶-benzylguanine)
CDI=N,N′-carbonyl diimidazole
CoA-SH=coenzyme A
DCC=dicyclohexylcarbodiimide
DCU=dicyclohexylurea
DIPEA=diisopropylethylamine
DMF=dimethylformamide
DMSO=dimethyl sulfoxide
DTT=dithiothreitol
EDC=1-(3-(dimethylamino)propyl)-3-ethylcarbodiimide
eq=equivalent
ESI-MS=electrospray ionization mass spectrometry
Et₃N=triethylamine
EtOAc=ethyl acetate
EtOH=ethanol
FAB-MS=fast atom bombardment mass spectrometry
HOBT=1-hydroxybenzotriazole
HPLC=high pressure liquid chromatography
Lys=lysine
MeNH₂=methylamine
MeOH=methanol
NHS=N-hydroxy succinimide
NMP=N-methylpyrrolidine
PEG12=—(CH₂CH₂O)₁₂—
PMe₃=trimethylphosphine
PYBOP=(benzotriazol-1-yloxy)-tripyrrolidino-phosphonium hexafluorophosphate
TFA=trifluoroacetic acid
Tris=tris(hydroxymethyl)methylamine

### Abbreviations for Molecular Biology Related Terms:

BG O6-Benzylguanine (derivative)
CT O2-Benzylcytosine (derivative)
LB Luria broth
TB terrific brith
IMAC Immbilized metal affinity chromatography
μ Mikro
M Milli
M Molar
mAk monoclonal antibody
Min Minute
mRNA (“messenger”) ribonucleic acid (RNA)
siRNA short interfering ribonucleic acid
DNA desoxyribonucleic acid
Mw molecular weight
N Nano
N-term Amino terminal (for proteins/oligo peptides)
C-term carboxy-terminal (for proteins/oligo peptides)
ORF open reading frame
PAA Polyacrylamid
PAGE Polyacrylamide gelelectrophoresis
pAk polyclonal antibody
PBS phosphate buffered saline
PBS-T PBS+0.05% (v/v) Tween-20
PCR polymerase chain reaction
PEG Polyethylenglycol
pelB bacterial leader-peptide for periplasmatic targeting in E. coli
RT reverse transkriptase
RT-PCR reverse transkriptase PCR
s Second
scFv single-chain variable fracment
SDS Natriumdodecylsulfat
Taq Thermus aquaticus
Tris Tris(hydroxymethyl)aminomethan
Tween 20 Polyoxyethylensorbitanmonolaurate
U Unit
o.n. over night
RPM rounds per minute
UV Ultra-violett
V Volt
v/v volume per volume
V_{H}/V_{L} variable region of heavy (H) or light (L) immunglobuline
Vol. Volume
W Watt
w/v weight per volume
scFv H22 Humanized scFv against human CD64
scFv 40 Murine antibody against apple scrap spores
CD40L natural ligand for CD40
CD30L natural ligand for CD30 murine scFv against human CD30
scFv Ki4
scFvKi3 murine scFv against human CD30
scFvKi2 murine scFv against human CD30
scFv 425 (Hai) murine scFv against human EGF receptor (EGFR)
hEGF Human epidermal growth factor binding to human EGF receptor
Adapter3 Adapter3 consists of an endosomal cleavable+membrane transfer peptide
scFv 14.1 murine scFv against pancreatic cancer cells
MOG Myelin Oligodendrocyte Glycoprotein
scFv35 human scFv against fetal acteylcholine receptor Trans-Activator of Transcription taken from HIV genome
TAT
scFvM12 human scFv against CEA (carcinoembryogenic antigen)
PIGF Phosphatidylinositol glycan, class F protein
VEGF Vascular endothelial growth factor
mSNAP SfiI restriction endonuclease recognition site depleted version of SNAP-Tag (SNAP 26m)
SNAP SNAP-Tag (SNAP26m/SNAP26b gene)
IL1-IL31 interleukin 1-interleukin 31
CXCL9 (MIG Chemokine CXC motif ligand 9
CXCL10 (IP10) Interferon-gamma-inducible protein 10
CXCL11 Chemokine CXC motif ligand 11
CXCL13 Chemokine CXC motif ligand 13
CXCL16 Chemokine CXC motif ligand 16
CCL11 (Eotaxin-1) Chemokine CC motif ligand 11
CCL14 Chemokine CC motif ligand 14
CCL16 Chemokine CC motif ligand 16
CCL18 Chemokine CC motif ligand 18
CCL27 Chemokine CC motif ligand 27
CCL28 Chemokine CC motif ligand 28
XCL1 (Lymphotatcin) Chemokine C motif ligand 1
CX3CL1(Neurotactin) Chemokine CX3C motif ligand 1
TGFbeta TGF beta receptor, type I
G-CSF Granulocyte-Colony Stimulating Factor
NGF Nerve growth factor
HGF Hepatocyte growth factor/scatter factor
sCD64 soluble CD64 (FC gamma receptor I)

### Example 1

### Chemical Synthesis of Tris{[2-(tert-butoxycarbonyl)ethoxy]methyl}methylamine (FIG. 1)

Tris(hydroxymethyl)methylamine (Tris, 2.42 g, 20.0 mmol) in 4.0 mL of a newly opened bottle of DMSO is cooled to 15.0° C. Then, 0.4 mL of 5.0 M NaOH is injected while stirring, followed by tert-butyl acrylate (10.0 mL, 68 mmol), which is injected dropwise. A solvent mixture of 5-10% water in DMSO is optimal for this reaction. The reaction mixture is allowed to reach room temperature and left stirring for 24 h. Then the crude mixture is poured onto water and extracted with ethyl acetate, the organic phase is dried over MgSO₄, and evaporated under reduced pressure to afford (FIG. 1). The compound is directly used for next step without further purification. FAB-MS: m/z 506 [M+H]⁺.

### Example 2

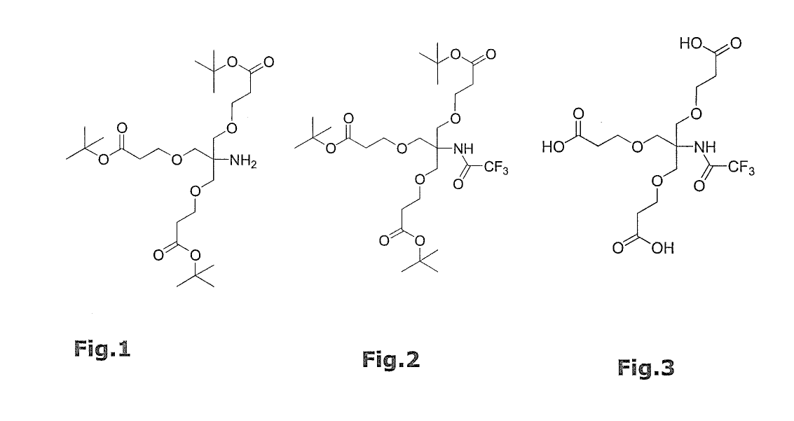

### Chemical Synthesis of N-Tris{[2-(tert-butoxycarbonyl)ethoxy]methyl}methyl trifluoroacetamide (FIG. 2)

To a solution of tris{[2-(tert-butoxycarbonyl)ethoxy]methyl}methylamine (FIG. 1) (10 mmol, 5.05 g) in MeOH (30 mL) is added triethylamine (1 eq, 10 mmol, 1.39 mL) at rt. Then, ethyl trifluoroacetate (1.3 eq, 13 mmol, 1.55 mL) is slowly added over 20 min at rt. The reaction mixture is stirred overnight at rt. Then, the solvent is evaporated, the residue is diluted with EtOAc (100 mL) and washed with a saturated solution of NaCl. The organic layer is dried over MgSO₄ and concentrated under reduce pressure. Flash chromatography (cyclohexane/EtOAc, 2/1→1/1) gives the desired compound (FIG. 2).

ESI-MS: m/z 602.31 [M+H]⁺.

### Example 3

### Chemical Synthesis of N-Tris{[2-(carboxy)ethoxy]methyl}methyl trifluoroacetamide (FIG. 3)

N-Tris{[2-(tert-butoxycarbonyl)ethoxy]methyl}methyl trifluoroacetamide (FIG. 2) (4.81 g, 8 mmol) is stirred in 80 mL of 96% formic acid for 18 h. Then, the formic acid is removed at reduced pressure at 50° C. to produce a colorless oil in quantitative yield.

ESI-MS: m/z 434.12 [M+H]⁺.

### Example 4

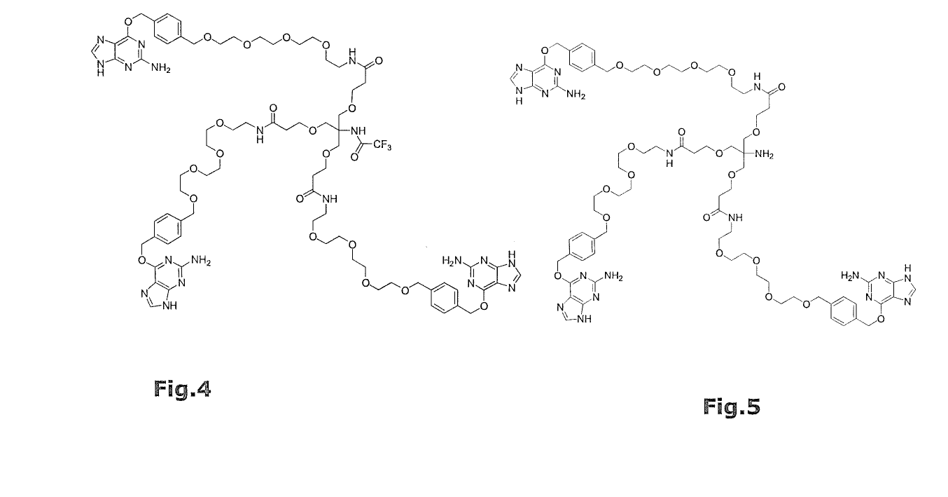

### Chemical Synthesis of N-Tris(BG-PEG4-NH-carbonylethyloxymethyl)methyl trifluoroacetamide (FIG. 4)

To a solution of (FIG. 3) (433 mg, 1 mmol, 1 eq) and BG-PEG4-NH2 (1.34 g, 3 mmol, 3 eq) in DMF (10 mL) are successively added DIPEA (495 μL, 3 mmol, 3 eq), HOBT (1 M in NMP, 3 mL, 3 mmol, 3 eq) and DCC (620 mg, 3 mmol, 3 eq) at rt. The resulting mixture is stirred overnight. Then the solvent is removed under reduced pressure and the mixture is diluted with 250 mL of EtOAc. The organic layer is washed with water, dried over MgSO₄ and evaporated under reduced pressure. Flash chromatography (CH₂Cl₂/MeOH, 10/1→5/1) gives the desired compound (FIG. 4). ESI-MS: m/z 1718.77 [M+H]⁺.

### Example 5

### Chemical Synthesis of Tris(BG-PEG4-NH-carbonylethyloxymethyl)methylamine (FIG. 5)

To a solution of compound (FIG. 4) (1.03 g, 0.6 mmol) in EtOH (15 mL) is added a solution of MeNH₂ (30% in EtOH, 30 mL). The corresponding solution is stirred overnight at rt. A cloudy mixture is obtained. The solid is removed by filtration and evaporation of the resulting clean solution affords the desired compound (FIG. 5). No further purification is required. ESI-MS: m/z 1621.79 [M+H]⁺.

### Example 6

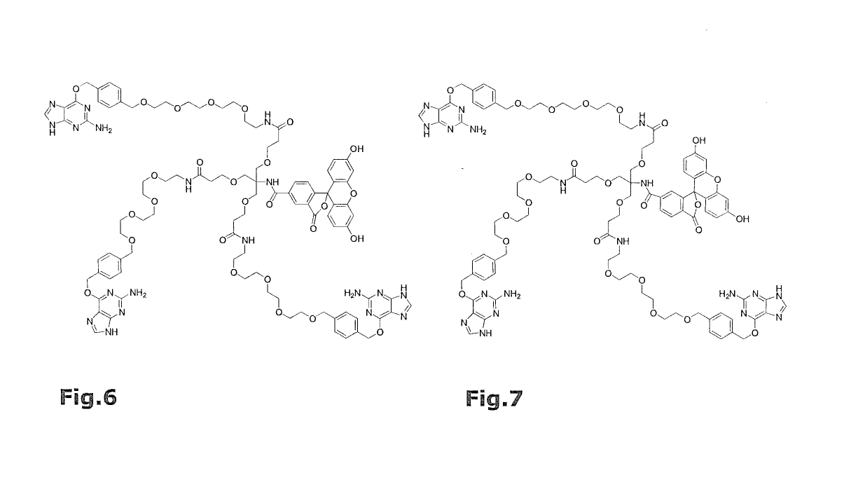

### Chemical Synthesis of N-Tris(BG-PEG4-NH-carbonylethyloxymethyl)methyl fluorescein-5-carboxamide (FIG. 6) and corresponding fluorescein-6-carboxamide (FIG. 7)

Compound (FIG. 5) (29 mg, 0.018 mmol) and 5(6)-carboxyfluorescein succinimidyl ester (8.5 mg, 0.018 mmol) are dissolved in 1 mL of DMF with Et₃N (2.7 μL, 0.018 mmol) and heated overnight at 31° C. The solvent is evaporated under vacuum and the compounds (FIG. 6) and (FIG. 7) isolated by reversed phase HPLC on a C18 column using a linear gradient of water:acetonitrile 95:5 to 20:80 in 20 min, 0.08% TFA). ESI-MS: m/z 1980.84 [M+H]⁺.

### Example 7

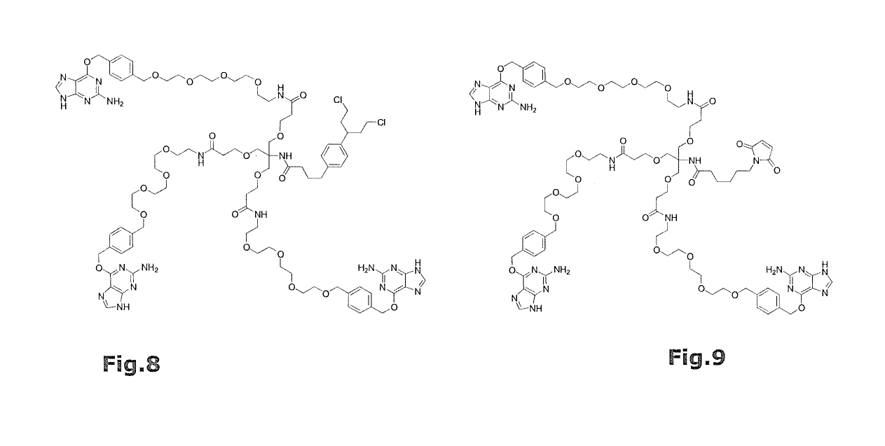

### Chemical Synthesis of N-Tris(BG-PEG4-NH-carbonylethyloxymethyl)methyl chlorambucil-carboxamide (FIG. 8)

To a solution of chlorambucil (22 mg, 0.072 mmol) in DMF (2 mL) is added PYBOP (38 mg, 0.072 mmol) at rt. The solution is stirred at room temperature for 20 min. Then, compound (FIG. 5) (116 mg, 0.072 mmol) and DIPEA (12 μL, 0.072 mmol) are added and the solution is heated at 50° C. for 5 min. The solution is stirred at room temperature overnight. Then the solvent is removed under reduced pressure and the mixture is diluted with 150 mL of EtOAc. The organic layer is washed with water, dried over MgSO₄ and evaporated under reduced pressure. Flash chromatography (CH₂Cl₂/MeOH, 10/1→5/1) gives the desired compound (FIG. 8).

ESI-MS: m/z 1906.86 [M+H]⁺.

### Example 8

### Chemical Synthesis of N-Tris(BG-PEG4-NH-carbonylethyloxymethyl)methyl 5-maleimidopentanecarboxamide (FIG. 9)

To a solution of 6-maleimido-hexanoic acid (8 mg, 0.036 mmol) in DMF (2 mL) is added PYBOP (19 mg, 0.036 mmol) at rt. The solution is stirred at room temperature for 20 min. Then compound (FIG. 5) (58 mg, 0.036 mmol) and DIPEA (6 μL, 0.036 mmol) are added and the solution is heated at 50° C. for 5 min. The solution is stirred at room temperature overnight. Then the solvent is removed under reduced pressure and the mixture is diluted with 150 mL of EtOAc. The organic layer is washed with water, dried over MgSO₄ and evaporated under reduced pressure. Flash chromatography (CH₂Cl₂/MeOH, 10/1→5/1) gives the desired compound (FIG. 9).

ESI-MS: m/z 1815.86 [M+H]⁺.

### Example 9

### Chemical Synthesis of Tris(BG-PEG4-NH-carbonylethyloxymethyl)methyl 6-maleimido-hexanoic amide siRNA Conjugate (FIG. 10)

5′-Thiol modified oligonucleotide (43 nmol) is reduced by incubation for 1 h at room temperature with 200 mM DTT in 200 μL Tris-buffer pH 8.5. The DTT is removed by gel filtration and the oligonucleotide eluted in PBS (pH 7.4). The most concentrated fractions are combined giving a total of 800 μL. 300 μL of a solution of compound (FIG. 9) (2.5 mM in DMF) is added and the reaction mixture incubated at room temperature for 1 h. The reaction mixture is diluted with water to a total volume of 2 mL and excess maleimide removed by gel filtration. The tris(BG-PEG4-NH-carbonylethyloxymethyl)methylamide-maleimide-oligonucleotide conjugate (FIG. 10) is then purified by HPLC (solvent A: 0.1 M tetraethylammonium acetate pH 6.9 in water; solvent B: acetonitrile).

### Example 10

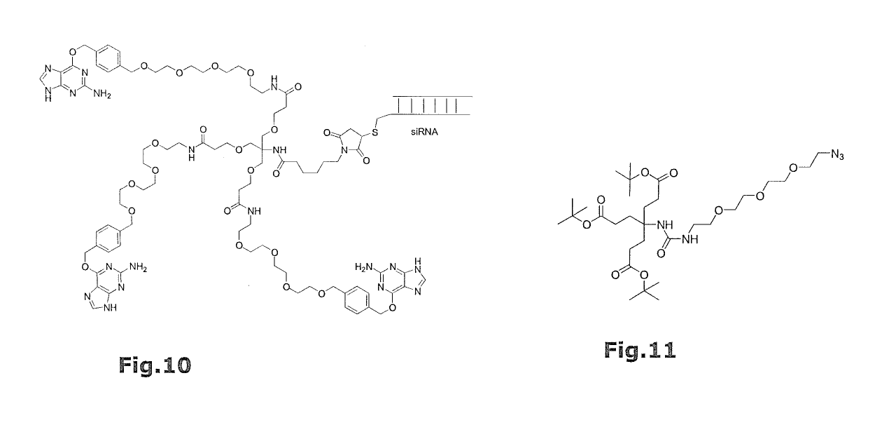

### Chemical Synthesis of N-2-(2-(2-(2-azidoethoxy)ethoxy)ethoxy)ethyl-N′-tris[2-(tert-butoxycarbonyl)ethyl]methyl-urea (FIG. 11)

To a solution of 11-azido-3,6,9-trioxaundecan-1-amine (1.55 g, 1 eq, 7.1 mmol) in DMF (2 mL) is added tris[2-(tert-butoxycarbonyl)ethyl]methyl isocyanate (3.1 g, 1 eq, 7.1 mmol) and Et₃N (988 μL, 1 eq, 7.1 mmol). The solution is stirred overnight at 31° C. Then the crude mixture is poured onto water and extracted with ethyl acetate, the organic phase is dried over MgSO₄, and evaporated under reduced pressure to afford (FIG. 11). No further purification is required.

FAB-MS: m/z 660.41 [M+H]⁺.

### Example 11

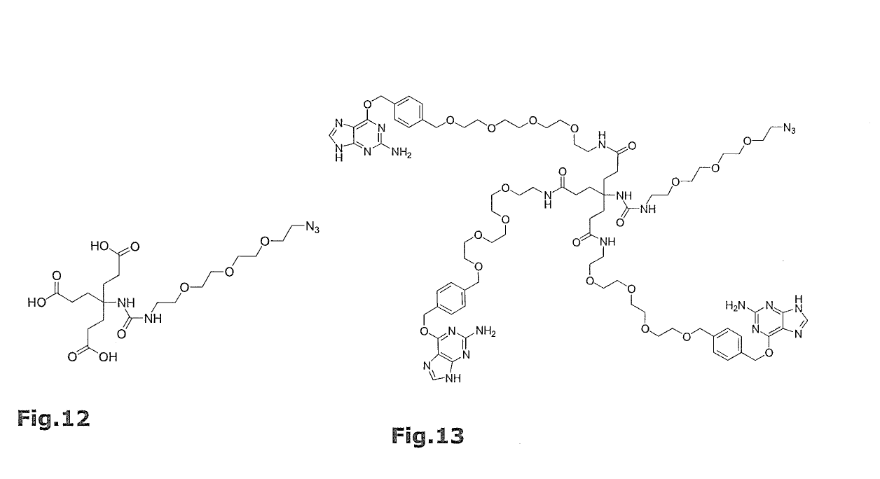

### Chemical Synthesis of 4-[2-carboxyethyl]-4-(2-(2-(2-(2-azidoethoxy)ethoxy)-ethoxy)ethylaminocarbonylamino)-1,7-heptanedicarboxylic acid (FIG. 12)

Compound (FIG. 11) (3.3 g, 5 mmol) is stirred in 50 mL of 96% formic acid for 18 h. Then, the formic acid is removed at reduced pressure at 50° C. to produce a colorless oil, compound (FIG. 12). ESI-MS: m/z 492.22 [M+H]⁺.

### Example 12

### Chemical Synthesis of N-Tris(BG-PEG4-NH-carbonylethyl)methyl-N′-2-(2-(2-(2-azidoethoxy)ethoxy)ethoxy)ethyl-urea (FIG. 13)

To a solution of compound (FIG. 12) (491 mg, 1 mmol, 1 eq) and BG-PEG4-NH2 (1.34 g, 3 mmol, 3 eq) in DMF (50 mL) are successively added DIPEA (495 μL, 3 mmol, 3 eq), HOBT (1 M in NMP, 3 mL, 3 mmol, 3 eq) and DCC (620 mg, 3 mmol, 3 eq) at rt. The resulting mixture is stirred overnight. Then the solvent is removed under reduced pressure and the mixture is diluted with 250 mL of EtOAc. The organic layer is washed with water, dried over MgSO₄ and evaporated under reduced pressure. Flash chromatography (CH₂Cl₂/MeOH, 10/1 5/1) gives the desired compound (FIG. 13). ESI-MS: m/z 1776.87 [M+H]⁺.

### Example 13

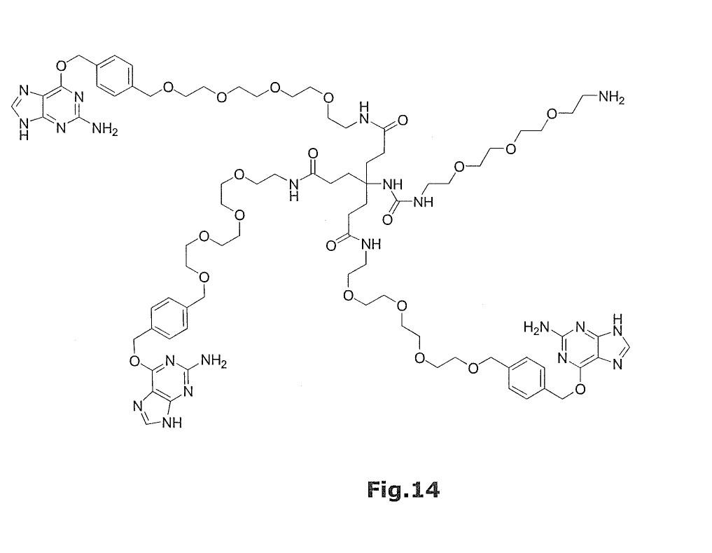

### Chemical Synthesis of N-Tris-(BG-PEG4-NH-carbonylethyl)methyl-N′-2-(2-(2-(2-aminoethoxy)ethoxy)ethoxy)ethyl-urea (FIG. 14)

To a solution of compound (FIG. 13) (708 mg, 0.4 mmol) in dioxane (10 mL) is added water (1 mL). Then PMe₃ (2.40 mL 1 M in THF solution, 6 eq) is added and the solution is stirred at room temperature for 2 h. The solvent is removed under reduced pressure, and the compound (FIG. 14) is obtained by purification with preparative HPLC. ESI-MS: m/z 1750.88 [M+H]⁺.

### Example 14

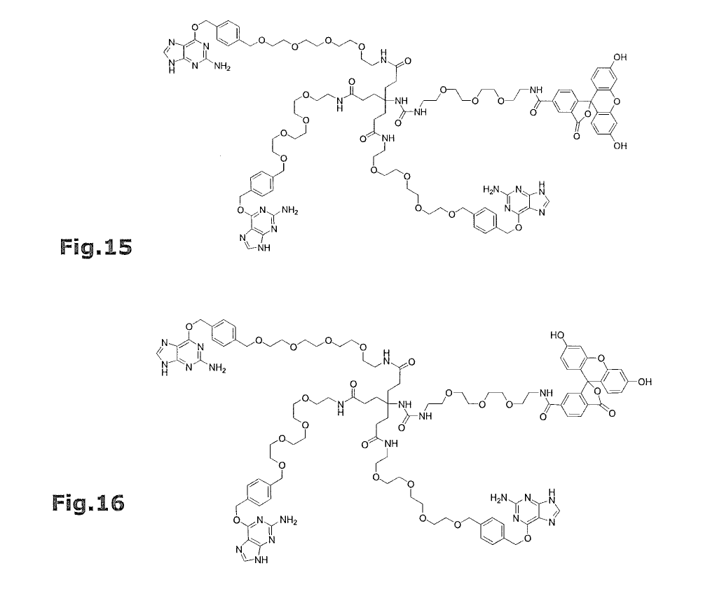

### Chemical Synthesis of N-Tris-(BG-PEG4-NH-carbonylethyl)methyl-N′-2-(2-(2-(2-fluorescein-5-carboxamido-ethoxy)ethoxy)ethoxy)ethyl-urea (FIG. 15) and Corresponding 6-fluorescein Derivative (FIG. 16)

Compound (FIG. 14) (18 mg, 0.01 mmol) and 5(6)-carboxyfluorescein succinimidyl ester (5 mg, 0.01 mmol) are dissolved in 800 μL DMF with Et₃N (1.6 μL, 0.01 mmol) and heated overnight at 31° C. The solvent is evaporated under vacuum and the compounds (FIG. 15) and (FIG. 16) isolated by reversed phase HPLC on a C18 column using a linear gradient of water:acetonitrile (from 95:5 to 20:80 in 20 min, 0.08% TFA). ESI-MS: m/z 2108.93 [M+H]⁺.

### Example 15

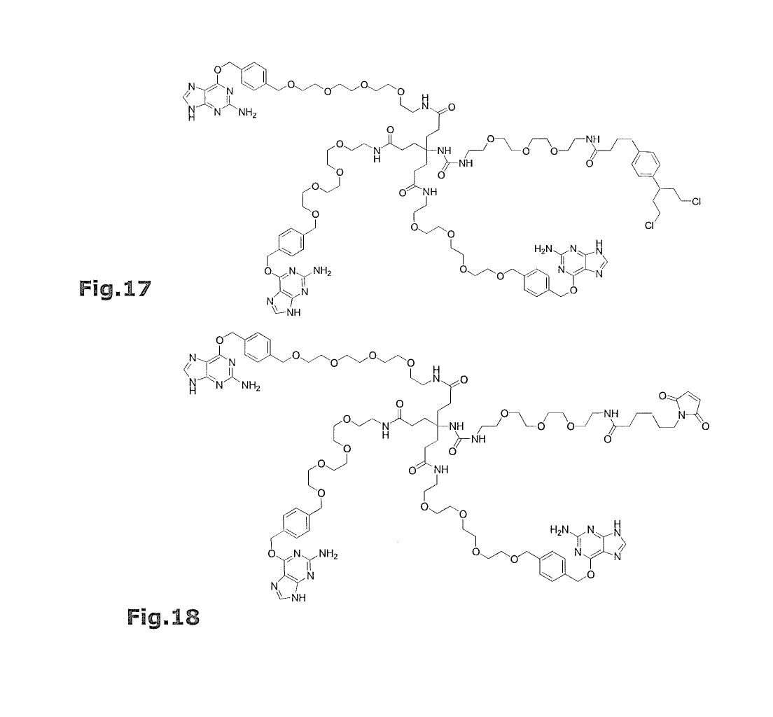

### Chemical Synthesis of N-Tris(BG-PEG4-NH-carbonylethyl)methyl-N′-2-(2-(2-(2-chlorambucil-carboxamido-ethoxy)ethoxy)ethoxy)ethyl-urea (FIG. 17)

To a solution of chlorambucil (18 mg, 0.06 mmol) in DMF (3 mL) is added PYBOP (31 mg, 0.06 mmol) at rt. The solution is stirred at room temperature for 20 min. Then compound (FIG. 14) (103 mg, 0.06 mmol) and DIPEA (10 μL, 0.06 mmol) are added and the solution is heated at 50° C. for 5 min. The solution is stirred at room temperature overnight. Then the solvent is removed under reduced pressure and the mixture is diluted with 150 mL of EtOAc. The organic layer is washed with water, dried over MgSO₄ and evaporated under reduced pressure. Flash chromatography (CH₂Cl₂/MeOH, 10/1□5/1) gives the desired compound (FIG. 17). ESI-MS: m/z 2050.99 [M+H]⁺.

### Example 16

### Chemical Synthesis of N-Tris(BG-PEG4-NH-carbonylethyl)methyl-N′-2-(2-(2-(2-(6-maleimido-hexanoylamido)ethoxy)ethoxy)ethoxy)ethyl-urea (FIG. 18)

To a solution of 6-maleimidohexanoic acid (10 mg, 0.046 mmol) in DMF (2 mL) is added PYBOP (24 mg, 0.046 mmol) at rt. The solution is stirred at room temperature for 20 min. Then compound (FIG. 14) (80 mg, 0.046 mmol) and DIPEA (7.7 μL, 0.046 mmol) are added and the solution is heated at 50° C. for 5 min. The solution is stirred at room temperature overnight. Then the solvent is removed under reduced pressure and the mixture is diluted with 150 mL of EtOAc. The organic layer is washed with water, dried over MgSO₄ and evaporated under reduced pressure. Flash chromatography (CH₂Cl₂/MeOH, 10/1 5/1) gives the desired compound (FIG. 18).

ESI-MS: m/z 1959.99 [M+H]⁺.

### Example 17

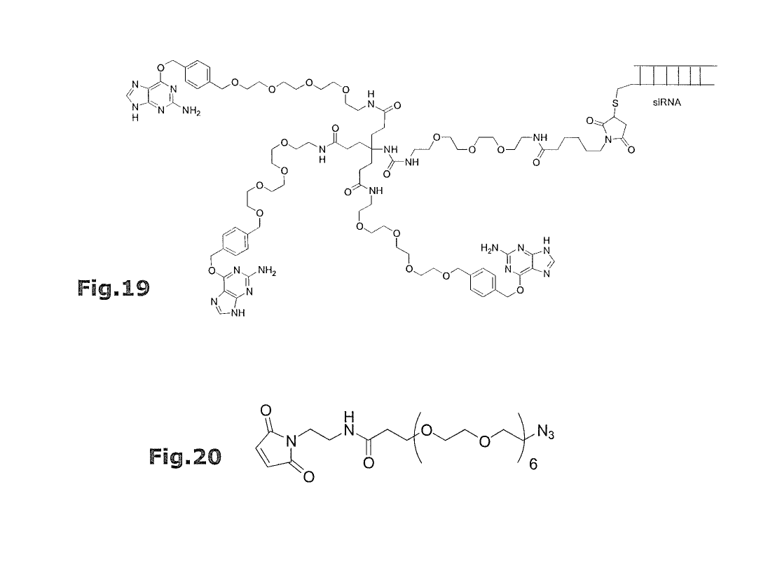

### Chemical Synthesis of N-Tris(BG-PEG4-NH-carbonylethyl)methyl-N′-2-(2-(2-(2-(6-maleimidohexanoylamido)ethoxy)ethoxy)ethoxy)ethyl-urea siRNA Conjugate (FIG. 19)

The 5′-thiol modified oligonucleotide (43 nmol) is reduced by incubation for 1 h at room temperature with 200 mM DTT in 200 μL Tris-buffer pH 8.5. The DTT is removed by gel filtration and the oligonucleotide eluted in PBS (pH 7.4). The most concentrated fractions are combined giving a total of 800 μL. 300 μL of a solution of compound (FIG. 18) (2.5 mM in DMF) is added and the reaction mixture incubated at room temperature for 1 h. The reaction mixture is diluted with water to a total volume of 2 mL and excess maleimide removed by gel filtration. The siRNA conjugate (FIG. 19) is then purified by HPLC (solvent A: 0.1 M tetraethylammonium acetate pH 6.9 in water; solvent B: acetonitrile).

### Example 18

### Chemical Synthesis of Azido-PEG12-propionic acid 2-maleimidoethylamide (FIG. 20)

N-(2-aminoethyl)maleimide trifluoroacetate (343 mg, 1.35 mmol) and azido-PEG12-propionic NHS ester (1 g, 1.35 mmol) are dissolved in 5 mL DMF with Et₃N (188 μL, 1.35 mmol) and heated overnight at 31° C. The solvent is evaporated under vacuum and the product is isolated by reversed phase HPLC on a C18 column using a linear gradient of water:acetonitrile (from 95:5 to 20:80 in 20 min, 0.08% TFA). ESI-MS: m/z 766.40 [M+H]⁺.

### Example 19

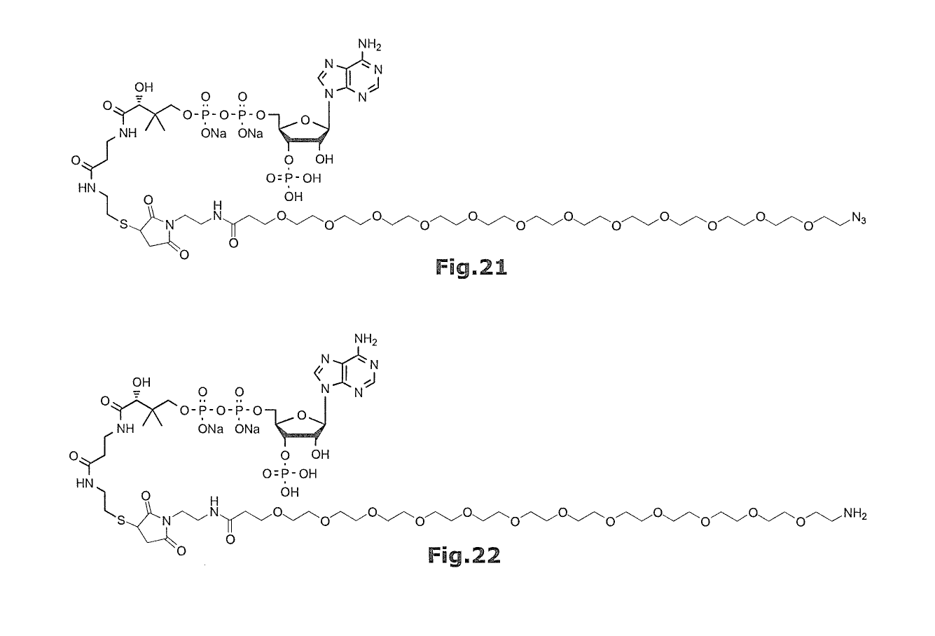

### Chemical Synthesis of Azido-PEG12-propionic acid 2-maleimidoethylamide CoA-SH Conjugate (FIG. 21)

A solution of maleimide derivative (FIG. 20) (192 mg, 1 eq, 252 μmol) in DMF (2 mL) is added to a solution of CoA-SH (248 mg, 1.2 eq, 304 μmol) in Tris-buffer (pH 7.5, 200 μL). The reaction mixture is shaken overnight at 31° C. Then the solvent is removed under vacuum and the crude mixture is purified via preparative HPLC. ESI-MS: m/z 1554.48 [M−Na]⁻.

### Example 20

### Chemical Synthesis of Amino-PEG12-propionic acid 2-maleimidoethylamide CoA-SH Conjugate (FIG. 22)

To a solution of compound (FIG. 21) (204 mg, 0.13 mmol) in dioxane (3 mL) is added water (450 μL). Then PMe₃ (800 μL 1 M in THF solution, 6 eq) is added and the solution is stirred at room temperature for 2 h. The solvent is removed under reduced pressure the compound is obtained by purification with preparative HPLC. ESI-MS: m/z 1527.48 [M−Na]⁻.

### Example 21

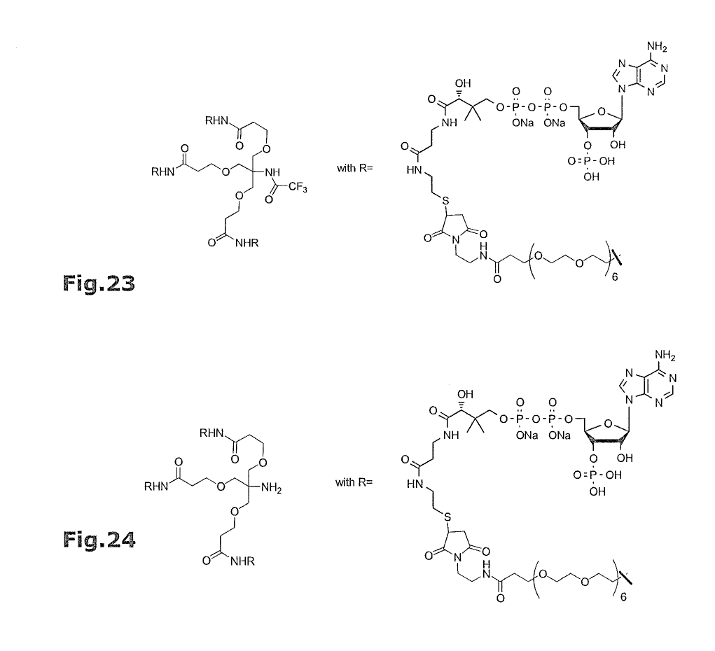

### Chemical Synthesis of N-Tris{2-(2-(2-(CoA-S-succinimido)ethylaminocarbonyl-PEG12)ethylaminocarbonyl)ethoxymethyl}methyl trifluoroacetamide (FIG. 23)

To a solution of (FIG. 3) (21 mg, 0.05 mmol, 1 eq) and (FIG. 22) (232 mg, 0.15 mmol, 3 eq) in DMF (1 mL) are successively added DIPEA (25 μL, 0.15 mmol, 3 eq), HOBT (1 M in NMP, 150 μL, 0.3 mmol, 3 eq) and DCC (31 mg, 0.15 mmol, 3 eq) at rt. The resulting mixture is stirred overnight. The solvent is removed under reduced pressure, and the compound (FIG. 23) is obtained by purification with preparative HPLC. ESI-MS: m/z 5010.4 [M−Na]⁻.

### Example 22

### Chemical Synthesis of N-Tris{2-(2-(2-(CoA-S-succinimido)ethylaminocarbonyl-PEG12)ethylaminocarbonyl)ethoxymethyl}methylamine (FIG. 24)

To a solution of compound (FIG. 21) (100 mg, 0.02 mmol) in EtOH (1.5 mL) is added a solution of MeNH₂ (3 mL, 30% in EtOH). The corresponding solution is stirred overnight at rt. Evaporation of the solvent affords the desired compound (FIG. 24). No further purification is required. ESI-MS: m/z 4914.4 [M−Na]⁻.

### Example 23

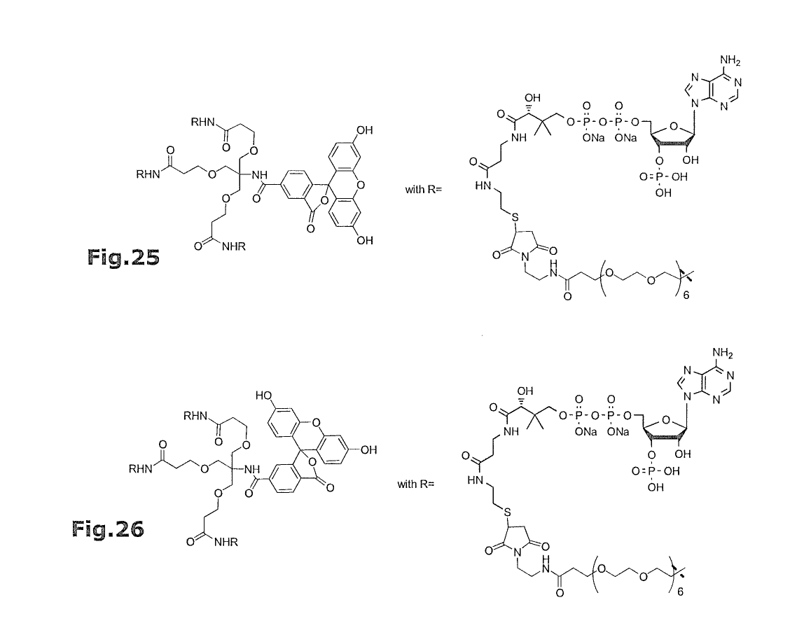

### Chemical Synthesis of N-Tris{2-(2-(2-(CoA-S-succinimido)ethylaminocarbonyl-PEG12)ethylaminocarbonyl)ethoxymethyl}methyl fluorescein-5-carboxamide (FIG. 25) and corresponding fluorescein-6-carboxamide (FIG. 26)

Compound (FIG. 24) (19 mg, 0.004 mmol) and 5(6)-carboxyfluorescein NHS ester (2 mg, 0.004 mmol) are dissolved in 600 μL DMF with Et₃N (0.6 μL, 0.004 mmol) and heated overnight at 31° C. The solvent is evaporated under vacuum and the compounds (FIG. 25) and (FIG. 26) isolated by reversed phase HPLC on a C18 column using a linear gradient of water:acetonitrile (from 95:5 to 20:80 in 20 min, 0.08% TFA). ESI-MS: m/z 5272.7 [M−Na]⁻.

### Example 24

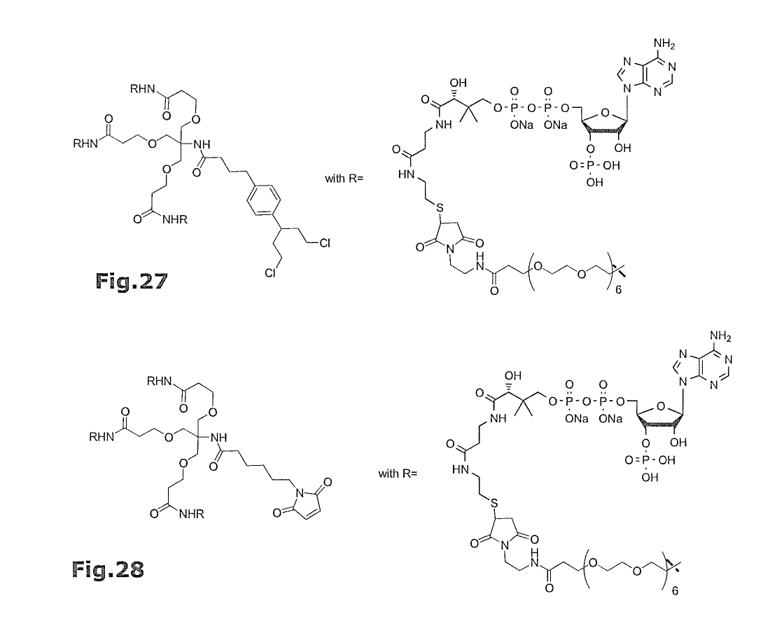

### Chemical Synthesis of N-Tris{2-(2-(2-(CoA-S-succinimido)ethylaminocarbonyl-PEG12)ethylaminocarbonyl)ethoxymethyl}methyl chlorambucil-carboxamide (FIG. 27)

To a solution of chlorambucil (1.8 mg, 0.006 mmol) in DMF (1 mL) is added PYBOP (3 mg, 0.006 mmol) at rt. The solution is stirred at room temperature for 20 min. Then compound (FIG. 24) (29 mg, 0.006 mmol) and DIPEA (0.9 μL, 0.006 mmol) are added and the solution is heated at 50° C. for 5 min. Then the solution is stirred at room temperature overnight. The solvent is removed under reduced pressure. Compound (FIG. 27) is isolated by reversed phase HPLC on a C18 column using a linear gradient of water:acetonitrile (from 95:5 to 20:80 in 20 min, 0.08% TFA).

ESI-MS: m/z 5200.6 [M−Na]⁻.

### Example 25

### Chemical Synthesis of N-Tris{2-(2-(2-(CoA-S-succinimido)-ethyl-aminocarbonyl-PEG12)ethylamino-carbonyl)ethoxymethyl}methyl 6-maleimido-hexanoyl-amide (FIG. 28)

To a solution of 6-maleimido-hexanoic acid (0.844 mg, 0.004 mmol) in DMF (1 mL) is added PYBOP (2.08 mg, 0.004 mmol) at rt. The solution is stirred at room temperature for 20 min. Then compound (FIG. 24) (19 mg, 0.004 mmol) and DIPEA (0.6 6 μL, 0.004 mmol) are added and the solution is heated at 50° C. for 5 min. The solution is stirred at room temperature overnight. Then the solvent is removed under reduced pressure. The compound is isolated by reversed phase HPLC on a C18 column using a linear gradient of water:acetonitrile (from 95:5 to 20:80 in 20 min, 0.08% TFA). ESI-MS: m/z 5107.7 [M−Na]⁻.

### Example 26

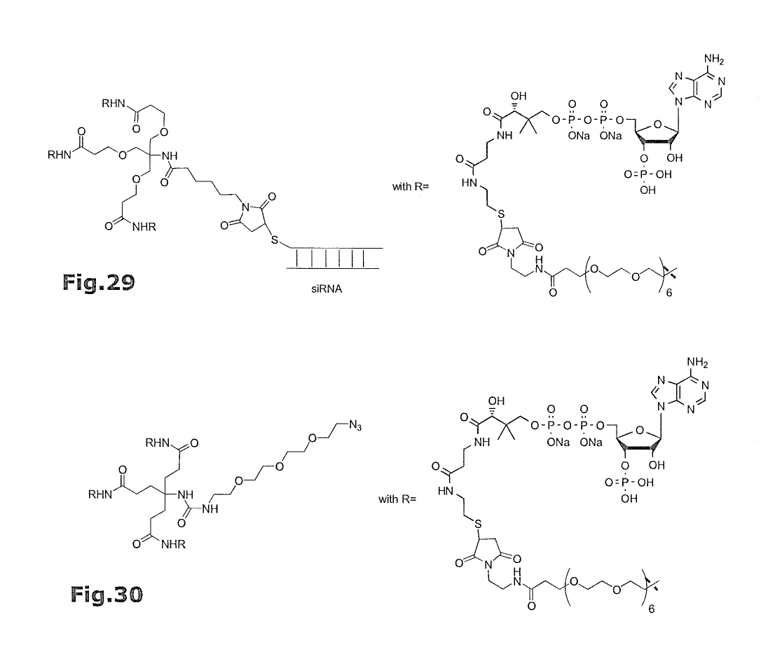

### Chemical Synthesis of N-Tris{2-(2-(2-(CoA-S-succinimido)ethylamino-carbonyl-PEG12)-ethylamino-carbonyl)ethoxymethyl}methyl 6-maleimidohexanoylamide siRNA Conjugate (FIG. 29)

The 5′-thiol modified oligonucleotide (43 nmol) is reduced by incubation for 1 h at room temperature with 200 mM DTT in 200 μL Tris-buffer pH 8.5. The DTT is removed by gel filtration and the oligonucleotide eluted in PBS (pH 7.4). The most concentrated fractions are combined giving a total of 800 μL. 300 μL of a solution of compound (FIG. 28) (2.5 mM in DMF) is added and the reaction mixture incubated at room temperature for 1 h. The reaction mixture is diluted with water to a total volume of 2 mL and excess maleimide removed by gel filtration. The conjugate (FIG. 29) is then purified by HPLC (solvent A: 0.1 M tetraethylammonium acetate pH 6.9 in water; solvent B: acetonitrile).

### Example 27

### Chemical Synthesis of N-Tris{2-(2-(2-(CoA-S-succinimido)ethylaminocarbonyl-PEG12)ethylaminocarbonyl)ethoxymethyl}methyl-N′-2-(2-(2-(2-azidoethoxy)-ethoxy)ethoxy)ethyl-urea (FIG. 30)

To a solution of compound (FIG. 12) (49 mg, 0.1 mmol, 1 eq) and compound (FIG. 22) (134 mg, 0.3 mmol, 3 eq) in DMF (5 mL) are successively added DIPEA (49 μL, 0.3 mmol, 3 eq), HOBT (1 M in NMP, 0.3 mL, 0.3 mmol, 3 eq) and DCC (62 mg, 0.3 mmol, 3 eq) at rt. The resulting mixture is stirred overnight. The solvent is removed under reduced pressure. The compound (FIG. 30) is isolated by reversed phase HPLC on a C18 column using a linear gradient of water:acetonitrile (from 95:5 to 20:80 in 20 min, 0.08% TFA). ESI-MS: m/z 5068.6 [M−Na]⁻.

### Example 28

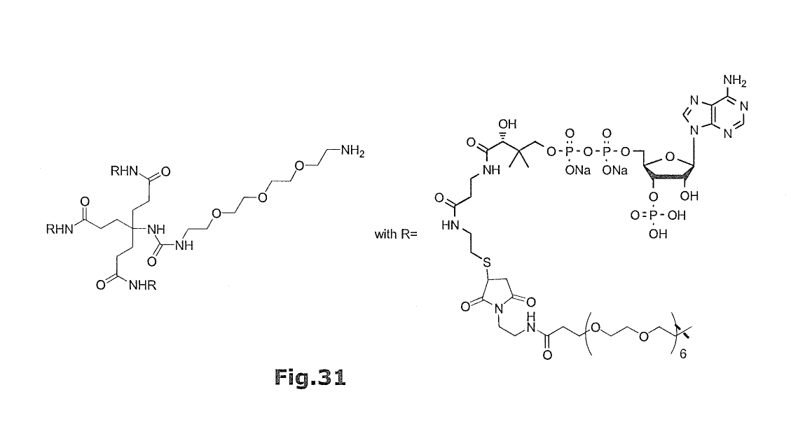

### Chemical Synthesis of N-Tris{2-(2-(2-(CoA-S-succinimido)ethylaminocarbonyl-PEG12)ethylaminocarbonyl)ethoxymethyl}methyl-N′-2-(2-(2-(2-aminoethoxy)-ethoxy)ethoxy)ethyl-urea (FIG. 31)

To a solution of compound (FIG. 30) (127 mg, 0.025 mmol) in dioxane (3 mL) is added water (450 μL). Then PMe₃ (154 μL of 1 M THF solution, 6 eq) is added and the solution is stirred at room temperature for 2 h. The solvent is removed under reduced pressure, and the compound (FIG. 31) is obtained by purification with preparative HPLC on a C18 column using a linear gradient of water:acetonitrile (from 95:5 to 20:80 in 20 min, 0.08% TFA). ESI-MS: m/z 5042.5 [M−Na]⁻.

### Example 29

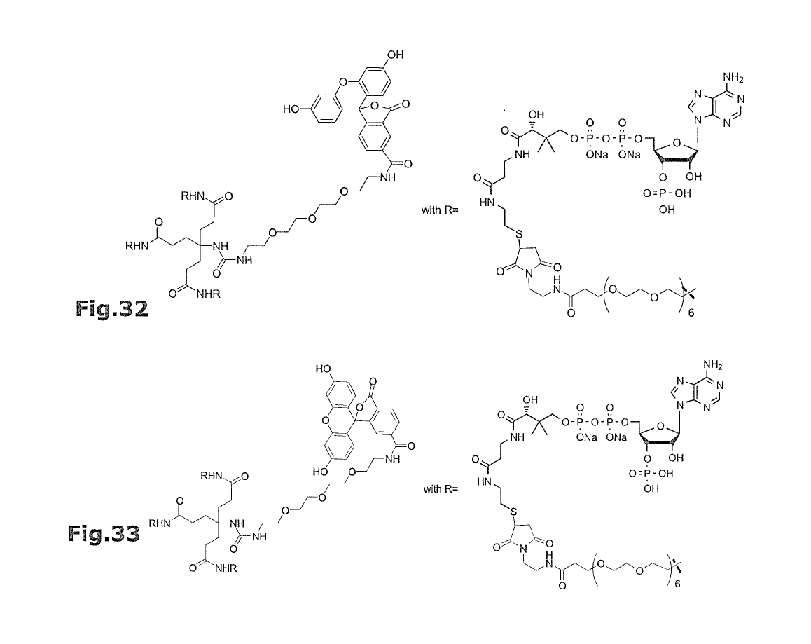

### Chemical Synthesis of N-Tris{2-(2-(2-(CoA-S-succinimido)ethyl-aminocarbonyl-PEG12)ethylaminocarbonyl)ethoxymethyl}methyl-N′-2-(2-(2-(2-fluorescein-5-carboxamidoethoxy)ethoxy)ethoxy)ethyl-urea (FIG. 32) and Corresponding 6-fluorescein Derivative (FIG. 33)

Compound (FIG. 31) (20 mg, 0.004 mmol) and 5(6)-carboxyfluorescein NHS ester (2 mg, 0.004 mmol) are dissolved in 600 μL DMF with Et₃N (0.6 μL, 0.004 mmol) and heated overnight at 31° C. The solvent is evaporated under vacuum and the compounds (FIG. 32) and (FIG. 33) isolated by reversed phase HPLC on a C18 column using a linear gradient of water:acetonitrile (from 95:5 to 20:80 in 20 min, 0.08% TFA). ESI-MS: m/z 5400.9 [M−Na]⁻.

### Example 30

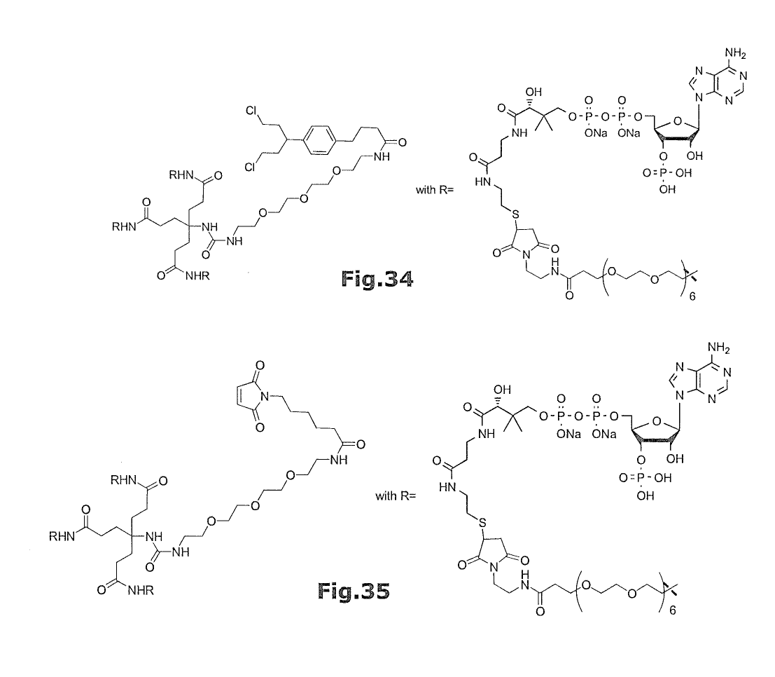

### Chemical Synthesis of N-Tris{2-(2-(2-(CoA-S-succinimido)ethylaminocarbonyl-PEG12)ethylaminocarbonyl)ethoxymethyl}methyl-N′-2-(2-(2-(2-chlorambucil-carboxamido-ethoxy)ethoxy)ethoxy)ethyl-urea (FIG. 34)

To a solution of chlorambucil (2.1 mg, 0.007 mmol) in DMF (1 mL) is added PYBOP (3.5 mg, 0.007 mmol) at rt. The solution is stirred at room temperature for 20 min. Then compound (FIG. 31) (35 mg, 0.007 mmol) and DIPEA (1.1 μL, 0.007 mmol) are added and the solution is heated at 50° C. for 5 min. The solution is stirred at room temperature overnight. Then the solvent is removed under reduced pressure. Compound (FIG. 34) is isolated by reversed phase HPLC on a C18 column using a linear gradient of water:acetonitrile (from 95:5 to 20:80 in 20 min, 0.08% TFA).

ESI-MS: m/z 5327.8 [M−Na]⁻.

### Example 31

### Chemical Synthesis of N-Tris-{2-(2-(2-(CoA-S-succinimido)ethylaminocarbonyl-PEG12)ethylaminocarbonyl)ethoxymethyl}methyl-N′-2-(2-(2-(2-(6-maleimido-hexanoylamido)ethoxy)ethoxy)ethoxy)ethyl-urea (FIG. 35)

To a solution of 6-maleimido-hexanoic acid (1 mg, 0.005 mmol) in DMF (1 mL) is added PYBOP (2.5 mg, 0.005 mmol) at rt. The solution is stirred at room temperature for 20 min. Then compound (FIG. 31) (24 mg, 0.005 mmol) and DIPEA (0.8 μL, 0.005 mmol) are added and the solution is heated at 50° C. for 5 min. The solution is stirred at room temperature overnight. Then the solvent is removed under reduced pressure. Compound (FIG. 35) is isolated by reversed phase HPLC on a C18 column using a linear gradient of water:acetonitrile (from 95:5 to 20:80 in 20 min, 0.08% TFA). ESI-MS: m/z 5235.7 [M−Na]⁻.

### Example 32

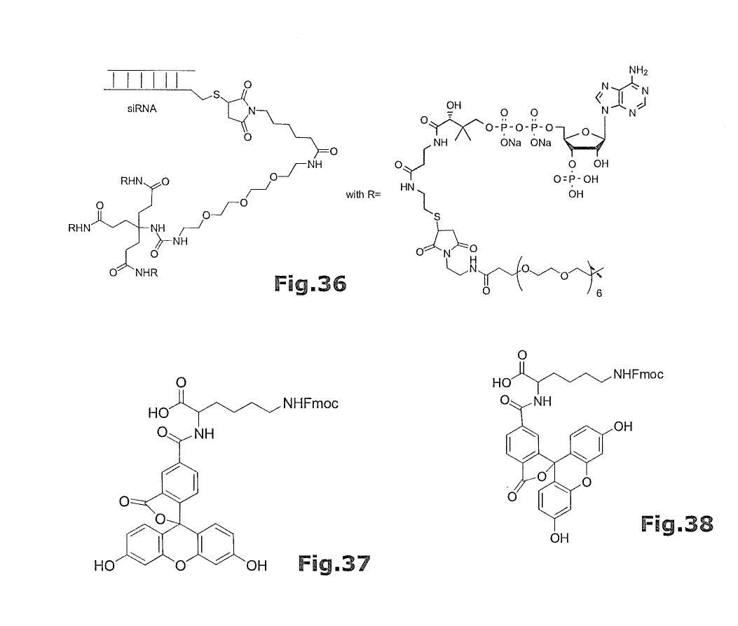

### Chemical Synthesis of N-Tris{2-(2-(2-(CoA-S-succinimido)ethylaminocarbonyl-PEG12)ethylaminocarbonyl)ethoxymethyl}methyl-N′-2-(2-(2-(2-(6-maleimido-hexanoylamido)ethoxy)ethoxy)ethoxy)ethyl-urea siRNA Conjugate (FIG. 36)

The 5′-thiol modified oligonucleotide (43 nmol) is reduced by incubation for 1 h at room temperature with 200 mM DTT in 200 μL Tris-buffer pH 8.5. The DTT is removed by gel filtration and the oligonucleotide eluted in PBS (pH 7.4). The most concentrated fractions are combined giving a total of 800 μL. 300 μL of a solution of compound (FIG. 35) (2.5 mM in DMF) is added and the reaction mixture incubated at room temperature for 1 h. The reaction mixture is diluted with water to a total volume of 2 mL, and excess maleimide removed by gel filtration. The conjugate (FIG. 36) is then purified by HPLC (solvent A: 0.1 M tetraethylammonium acetate pH 6.9 in water; solvent B: acetonitrile).

### Example 33

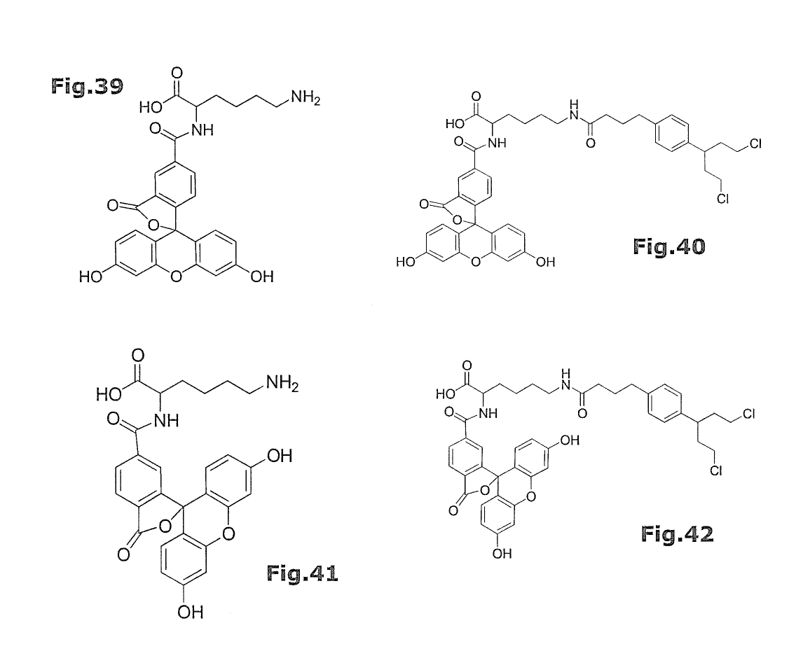

### Chemical Synthesis of 5-Fluorescein-Lys-Fmoc-OH (FIG. 37) and 6-fluorescein-Lys-Fmoc-OH (FIG. 38)

Fmoc-Lys-OH (184 mg, 0.5 mmol) and 5(6)-carboxyfluorescein NHS ester (237 mg, 0.5 mmol) are dissolved in 5 mL of DMF with Et₃N (70 μL, 0.5 mmol) and heated overnight at 31° C. Then the crude mixture is poured onto water (100 mL). The aqueous is basified (pH=9) with NaOH (1 M). The aqueous phase is washed with ethyl acetate. Upon acidification of the aqueous phase with acetic acid, a yellowish precipitate is formed. The solid is collected via filtration to afford the desired compound as a mixture of isomers (FIG. 37) and (FIG. 38).

ESI-MS: m/z 727.7 [M+H]⁺.

### Example 34

### Chemical Synthesis of 5-Fluorescein-Lys-OH (FIG. 39) and 6-fluorescein-Lys-OH (FIG. 40)

To a solution of mixture of compounds (FIG. 37) and (FIG. 38) (300 mg, 0.4 mmol) in DMF (3 mL) is added diethylamine (600 μL) at rt. The solution is stirred at room temperature for 3 h. The solvent is removed under reduced pressure and the desired mixture of compounds (FIG. 39) and (FIG. 40) is directly used for next step.

ESI-MS: m/z 505.15 [M+H]⁺.

### Example 35

### Chemical Synthesis N-5-Fluorescein-N′-chlorambucil-Lys-OH (FIG. 41) and N-6-fluorescein-N′-chlorambucil-Lys-OH (FIG. 42)

To a solution of chlorambucil (106 mg, 0.35 mmol) in DMF (3 mL) is added PYBOP (182 mg, 0.35 mmol) at rt. The solution is stirred at room temperature for 20 min. Then the mixture of isomers (FIG. 39) and (FIG. 40) (176 mg, 0.35 mmol) and DIPEA (58 μL, 0.35 mmol) are added and the solution is heated at 50° C. for 5 min. The solution is stirred at room temperature overnight. Then the crude mixture is poured onto water (60 mL). The aqueous solution is basified (pH=9) with NaOH (1 M). The aqueous phase is washed with ethyl acetate. Upon acidification of the aqueous phase with acetic acid, a yellowish precipitate is formed. The solid is collected via filtration to afford the desired compound as a mixture of isomers (FIG. 41) and (FIG. 42).

ESI-MS: m/z 789.23 [M+H]⁺.

### Example 36

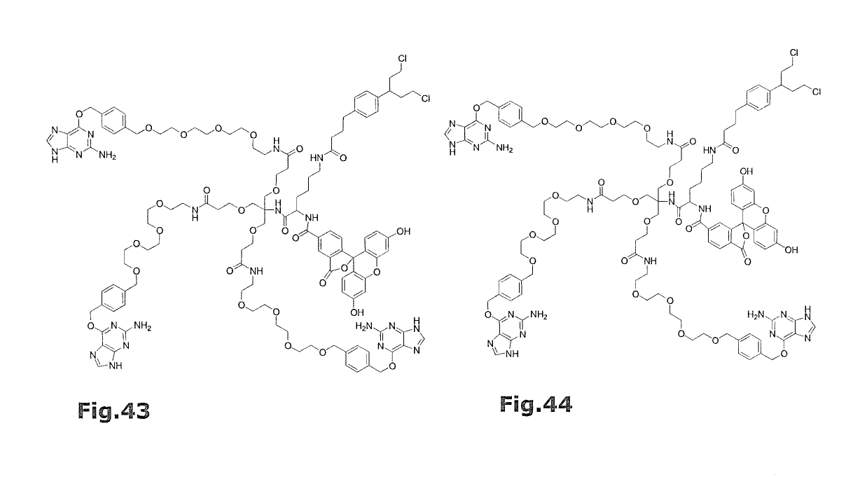

### Chemical Synthesis of N-Tris(BG-PEG4-NH-carbonylethyloxymethyl)-methyl N′-5-fluorescein-N″-chlorambucil-Lys-amide (FIG. 43) and Corresponding 6-fluorescein Derivative (FIG. 44)

To a solution of a mixture of isomers (FIG. 41) and (FIG. 42) (15 mg, 0.02 mmol) in DMF (2 mL) is added PYBOP (10 mg, 0.02 mmol) at rt. The solution is stirred at room temperature for 20 min. Then compound (FIG. 5) (32 mg, 0.02 mmol) and DIPEA (3.3 μL, 0.02 mmol) are added and the solution is heated at 50° C. for 5 min. The solution is stirred at room temperature overnight. The solution is poured onto water, the precipitate is filtered and washed with water. The desired compounds (FIG. 43) and (FIG. 44) are obtained as a solid. ESI-MS: m/z 2393.01 [M+H]⁺.

### Example 37

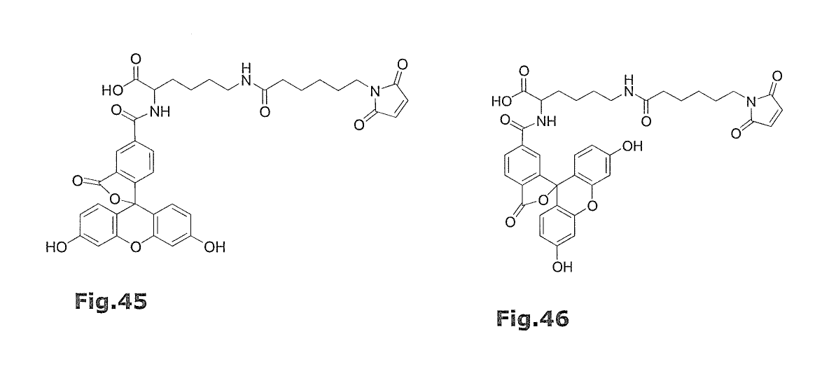

### Chemical Synthesis of N-5-Fluorescein-N′-6-maleimidohexanoyl-Lys-OH (FIG. 45) and N-6-fluorescein-N′-6-maleimidohexanoyl-Lys-OH (FIG. 46)

To a solution of 6-maleimido-hexanoic acid (66 mg, 0.31 mmol) in DMF (3 mL) is added PYBOP (161 mg, 0.31 mmol) at rt. The solution is stirred at room temperature for 20 min. Then the mixture of compounds (FIG. 39) and (FIG. 40) (156 mg, 0.31 mmol) and DIPEA (51 μL, 0.31 mmol) is added and the solution is heated at 50° C. for 5 min. The solution is stirred at room temperature overnight. Then the crude mixture is poured onto water (60 mL). The aqueous is basified (pH=9) with NaOH (1 M). The aqueous phase is washed with ethyl acetate. Upon acidification of the aqueous phase with acetic acid, a yellowish precipitate is formed. The solid is collected via filtration to afford the desired compound as a mixture of isomers (FIG. 45) and (FIG. 46). ESI-MS: m/z 699.23 [M+H]⁺.

### Example 38

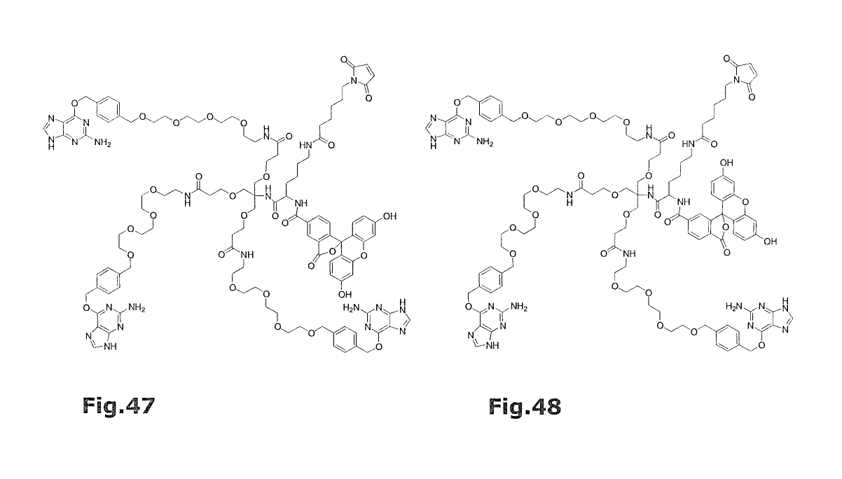

### Chemical synthesis of N-Tris(BG-PEG4-NH-carbonylethyloxymethyl)methyl N′-5-fluorescein-N″-6-maleimidohexanoyl-Lys-amide (FIG. 47) and Corresponding 6-fluorescein Derivative (FIG. 48)

To a solution of mixture of isomers (FIG. 45) and (FIG. 46) (9 mg, 0.013 mmol) in DMF (2 mL) is added PYBOP (6.5 mg, 0.013 mmol) at rt. The solution is stirred at room temperature for 20 min. Then compound (FIG. 5) (21 mg, 0.013 mmol) and DIPEA (2.1 μL, 0.013 mmol) are added and the solution is heated at 50° C. for 5 min. The solution is stirred at room temperature overnight. The solution is poured onto water, the precipitate is filtered and washed with water. The desired compound is obtained as a mixture of isomers (FIG. 47) and (FIG. 48) as a solid.

ESI-MS: m/z 2302.01 [M+H]⁺.

### Example 39

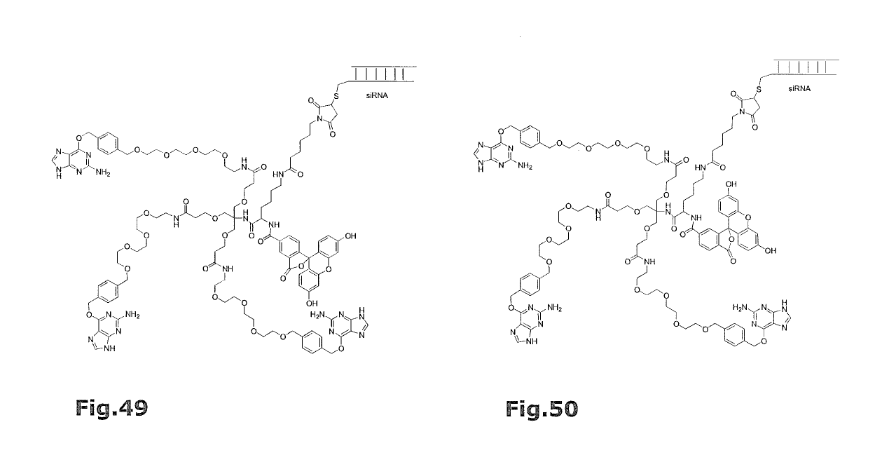

### Chemical Synthesis of N-Tris(BG-PEG4-NH-carbonylethyloxymethyl)methyl N′-5-fluorescein-N″-6-maleimidohexanoyl-Lys-amide siRNA Conjugate (FIG. 49) and Corresponding 6-fluorescein Derivative (FIG. 50)

The 5′-thiol modified oligonucleotide (43 nmol) is reduced by incubation for 1 h at room temperature with 200 mM DTT in 200 μL Tris-buffer pH 8.5. The DTT is removed by gel filtration and the oligonucleotide eluted in PBS (pH 7.4). The most concentrated fractions are combined giving a total of 800 μL. 300 μL solution of a mixture of isomers (FIG. 47) and (FIG. 48) (2.5 mM in DMF) is added and the reaction mixture incubated at room temperature for 1 h. The reaction mixture is diluted with water to a total volume of 2 mL and excess maleimide removed by gel filtration. The mixture of conjugates (FIG. 49) and (FIG. 50) is then purified by HPLC (solvent A: 0.1 M tetraethylammonium acetate pH 6.9 in water; solvent B: acetonitrile).

### Example 40

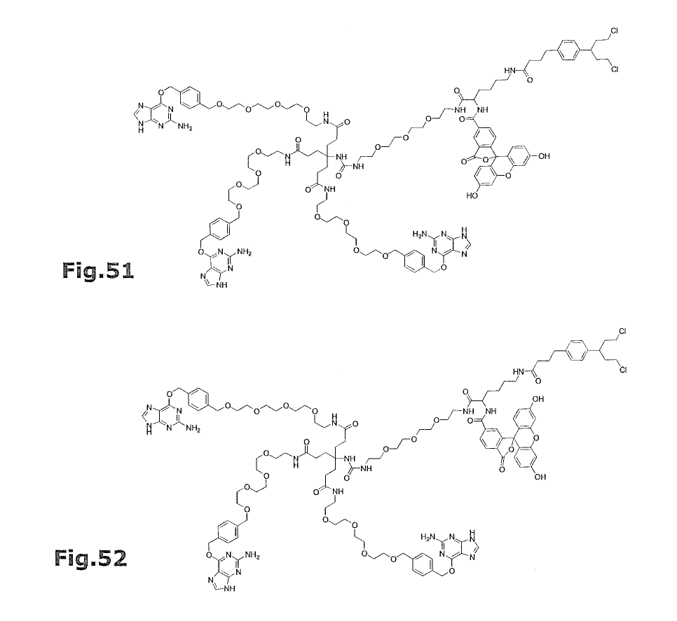

### Chemical Synthesis of N-Tris(BG-PEG4-NH-carbonylethyloxymethyl)methyl N′-2-(2-(2-(2-(N″-5-fluorescein-N′″-chlorambucil-Lys-amido)ethoxy)ethoxy)ethoxy)-ethyl-urea (FIG. 51) and Corresponding 6-fluorescein Derivative (FIG. 52)

To a solution of mixture of isomers (FIG. 41) and (FIG. 42) (19 mg, 0.024 mmol) in DMF (3 mL) is added PYBOP (13 mg, 0.024 mmol) at rt. The solution is stirred at room temperature for 20 min. Then compound (FIG. 14) (42 mg, 0.024 mmol) and DIPEA (4 μL, 0.024 mmol) are added and the solution is heated at 50° C. for 5 min. The solution is stirred at room temperature overnight. The solution is poured onto water, the precipitate is filtered and washed with water. The desired compound is obtained as a mixture of isomers (FIG. 51) and (FIG. 52).

ESI-MS: m/z 2521.10 [M+H]⁺.

### Example 41

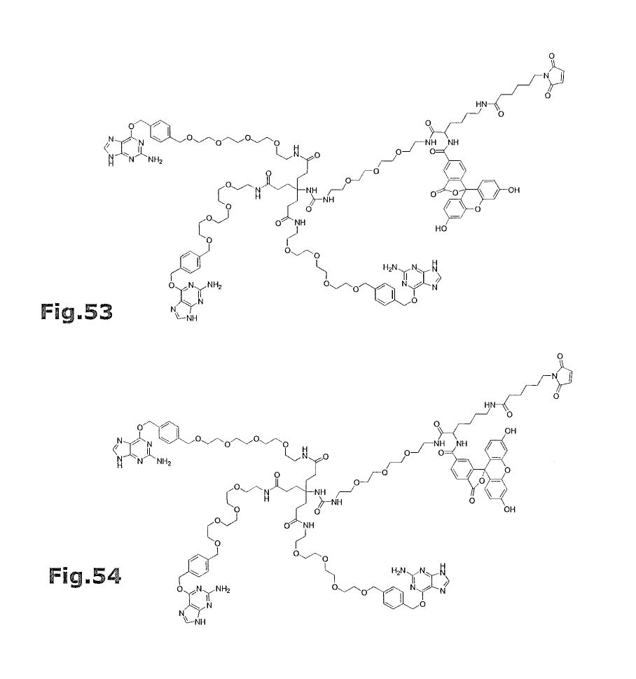

### Chemical Synthesis of N-Tris(BG-PEG4-NH-carbonylethyloxymethyl)methyl N′-2-(2-(2-(2-(N″-5-fluorescein-N′″-6-maleimidohexanoyl-Lys-amido)ethoxy)ethoxy)ethoxy)ethyl-urea (FIG. 53) and Corresponding 6-fluorescein Derivative (FIG. 54)

To a solution of a mixture of isomers (FIG. 45) and (FIG. 46) (21 mg, 0.03 mmol) in DMF (3 mL) is added PYBOP (16 mg, 0.03 mmol) at rt. The solution is stirred at room temperature for 20 min. Then compound (FIG. 14) (53 mg, 0.03 mmol) and DIPEA (5 μL, 0.03 mmol) are added and the solution is heated at 50° C. for 5 min. The solution is stirred at room temperature overnight. The solution is poured onto water, the precipitate is filtered and washed with water. The desired compound is obtained as a mixture of isomers (FIG. 53) and (FIG. 54).

ESI-MS: m/z 2430.10 [M+H]⁺.

### Example 42

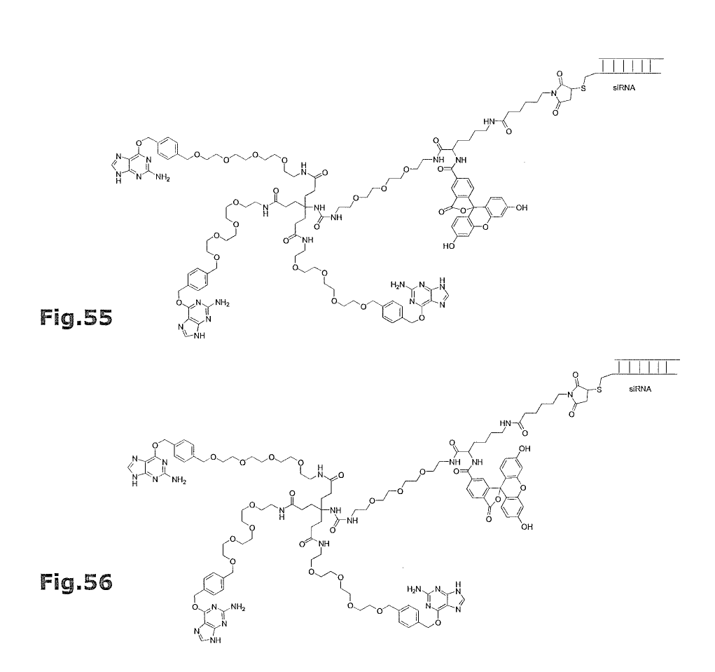

### Chemical Synthesis of N-Tris(BG-PEG4-NH-carbonylethyloxymethyl)methyl N′-2-(2-(2-(2-(N″-5-fluorescein-N′″-6-maleimidohexanoyl-Lys-amido)ethoxy)ethoxy)-ethoxy)ethyl-urea siRNA Conjugate (FIG. 55) and Corresponding 6-fluorescein Derivative (FIG. 56)

The 5′-thiol modified oligonucleotide (43 nmol) is reduced by incubation for 1 h at room temperature with 200 mM DTT in 200 μL Tris-buffer pH 8.5. The DTT is removed by gel filtration and the oligonucleotide eluted in PBS (pH 7.4). The most concentrated fractions are combined giving a total of 800 μL. 300 μL solution of a mixture of isomers (FIG. 53) and (FIG. 54) (2.5 mM in DMF) is added and the reaction mixture incubated at room temperature for 1 h. The reaction mixture is diluted with water to a total volume of 2 mL, and excess maleimide removed by gel filtration. The mixture of conjugates (FIG. 55) and (FIG. 56) is then purified by HPLC (solvent A: 0.1 M tetraethylammonium acetate pH 6.9 in water; solvent B: acetonitrile).

### Example 43

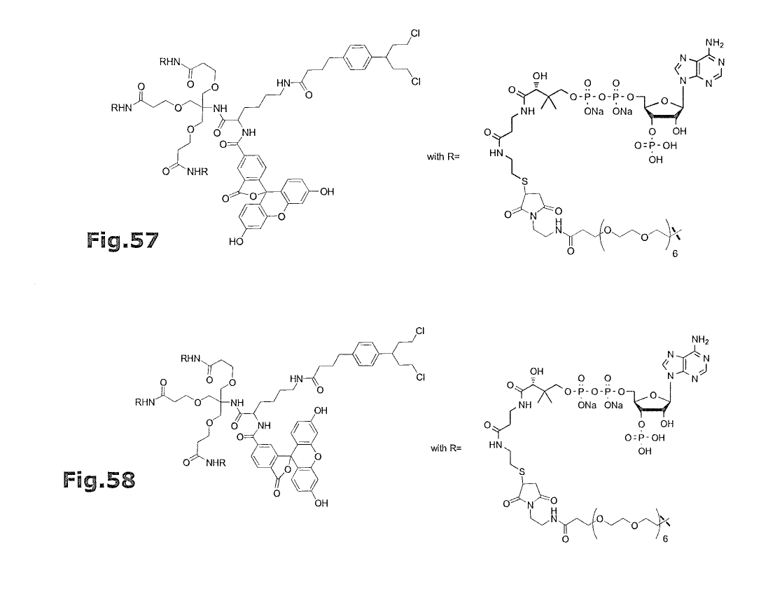

### Chemical Synthesis of N-Tris-{2-(2-(2-(CoA-S-succinimido)ethylaminocarbonyl-PEG12)ethylaminocarbonyl)ethoxymethyl}-methyl N′-5-fluorescein-N″-chlorambucil-Lys-amide (FIG. 57) and Corresponding 6-fluorescein Derivative (FIG. 58)

To a solution of mixture of isomers (FIG. 41) and (FIG. 42) (12 mg, 0.015 mmol) in DMF (2 mL) is added PYBOP (8 mg, 0.015 mmol) at rt. The solution is stirred at room temperature for 20 min. Then compound (FIG. 24) (73 mg, 0.015 mmol) and DIPEA (2.5 μL, 0.015 mmol) are added and the solution is heated at 50° C. for 5 min. The solution is stirred at room temperature overnight. The solvent is removed under reduced pressure, and the compound is obtained as a mixture of isomers (FIG. 57) and (FIG. 58) by purification with preparative HPLC.

ESI-MS: m/z 5686.1 [M−Na]⁻.

### Example 44

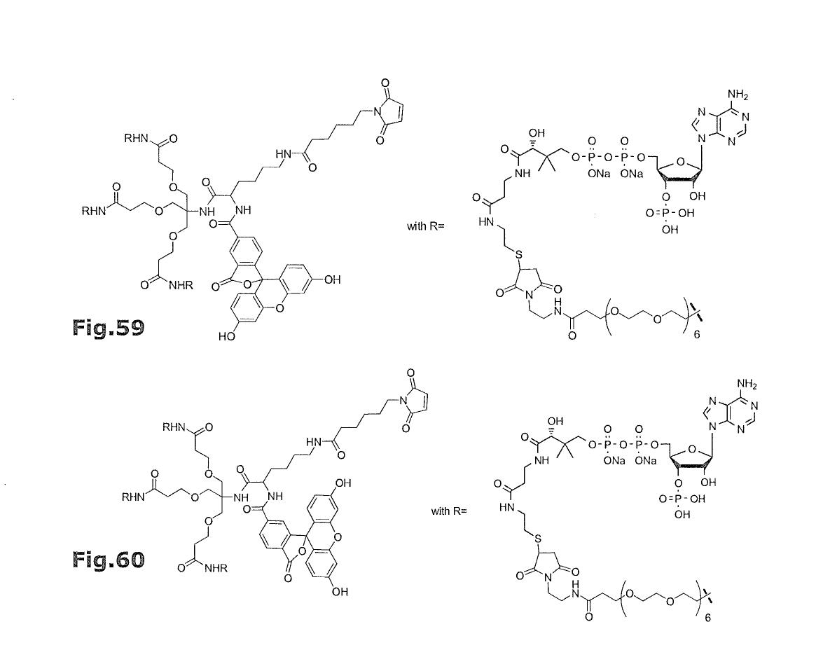

### Chemical Synthesis of N-Tris{2-(2-(2-(CoA-S-succinimido)ethylaminocarbonyl-PEG12)ethylaminocarbonyl)ethoxymethyl}methyl N′-5-fluorescein-N″-6-maleimido-hexanoyl-Lys-amide (FIG. 59) and Corresponding 6-fluorescein Derivative (FIG. 60)

To a solution of mixture of isomers (FIG. 45) and (FIG. 46) (7 mg, 0.01 mmol) in DMF (2 mL) is added PYBOP (5 mg, 0.01 mmol) at rt. The solution is stirred at room temperature for 20 min. Then compound (FIG. 24) (50 mg, 0.01 mmol) and DIPEA (1.65 μL, 0.01 mmol) are added and the solution is heated at 50° C. for 5 min. The solution is stirred at room temperature overnight. The solvent is removed under reduced pressure, and the compound is obtained as a mixture of isomers (FIG. 59) and (FIG. 60) by purification with preparative HPLC.

ESI-MS: m/z 5594.1 [M−Na]⁻.

### Example 45

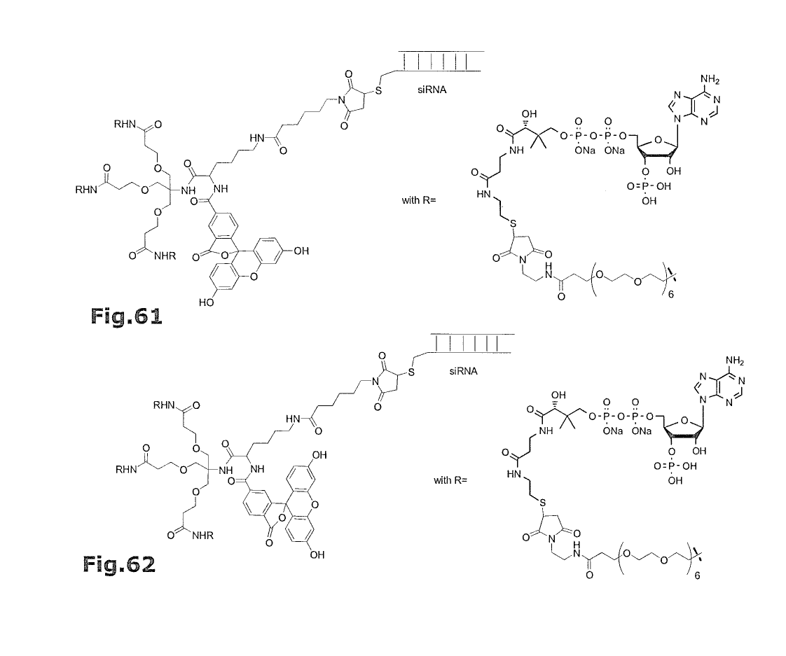

### Chemical Synthesis of N-Tris{2-(2-(2-(CoA-S-succinimido)ethylaminocarbonyl-PEG12)ethylaminocarbonyl)ethoxymethyl}methyl N′-5-fluorescein-N″-6-maleimido-hexanoyl-Lys-amide siRNA Conjugate (FIG. 61) and Corresponding 6-fluorescein Derivative (FIG. 62)

The 5′-thiol modified oligonucleotide (43 nmol) is reduced by incubation for 1 h at room temperature with 200 mM DTT in 200 μL Tris-buffer pH 8.5. The DTT is removed by gel filtration and the oligonucleotide eluted in PBS (pH 7.4). The most concentrated fractions are combined giving a total of 800 μL. 300 μL of a solution of mixture of isomers (FIG. 59) and (FIG. 60) (2.5 mM in DMF) is added and the reaction mixture incubated at room temperature for 1 h. The reaction mixture is diluted with water to a total volume of 2 mL, and excess maleimide removed by gel filtration. The mixture of conjugates (FIG. 61) and (FIG. 62) is then purified by HPLC (solvent A: 0.1 M tetraethylammonium acetate pH 6.9 in water; solvent B: acetonitrile).

### Example 46

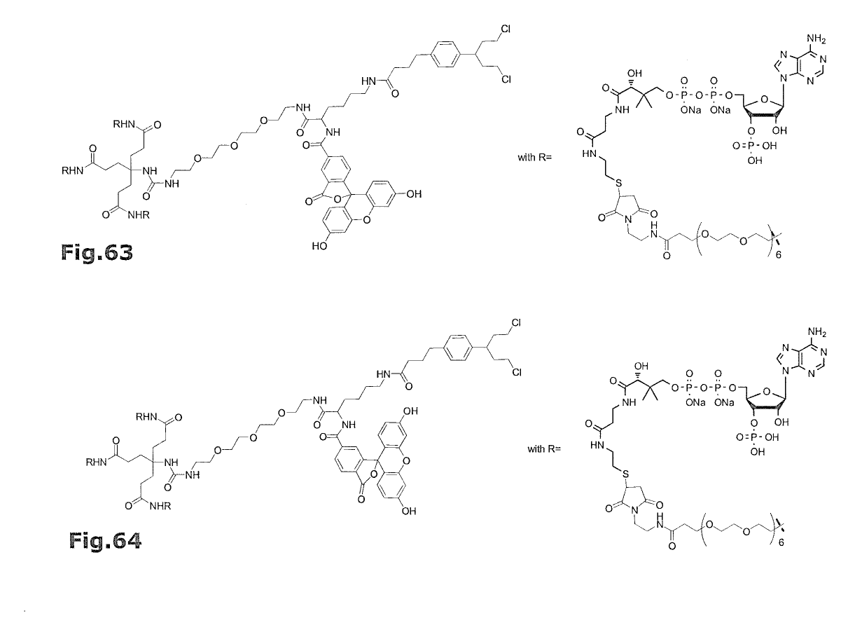

### Chemical Synthesis of N-Tris{2-(2-(2-(CoA-S-succinimido)ethylaminocarbonyl-PEG12)ethylaminocarbonyl)ethoxymethyl}methyl N′-2-(2-(2-(2-(N″-5-fluorescein-N′″-chlorambucil-Lys-amido)ethoxy)ethoxy)ethoxy)ethyl-urea (FIG. 63) and Corresponding 6-fluorescein Derivative (FIG. 64)

To a solution of mixture of isomers (FIG. 41) and (FIG. 42) (5 mg, 0.006 mmol) in DMF (1 mL) is added PYBOP (3 mg, 0.006 mmol) at rt. The solution is stirred at room temperature for 20 min. Then compound (FIG. 31) (30 mg, 0.006 mmol) and DIPEA (1 μL, 0.006 mmol) are added and the solution is heated at 50° C. for 5 min. The solution is stirred at room temperature overnight. The solvent is removed under reduced pressure, and the compound is obtained as a mixture of isomers (FIG. 63) and (FIG. 64) by purification with preparative HPLC.

ESI-MS: m/z 5814.9 [M−Na]⁻.

### Example 47

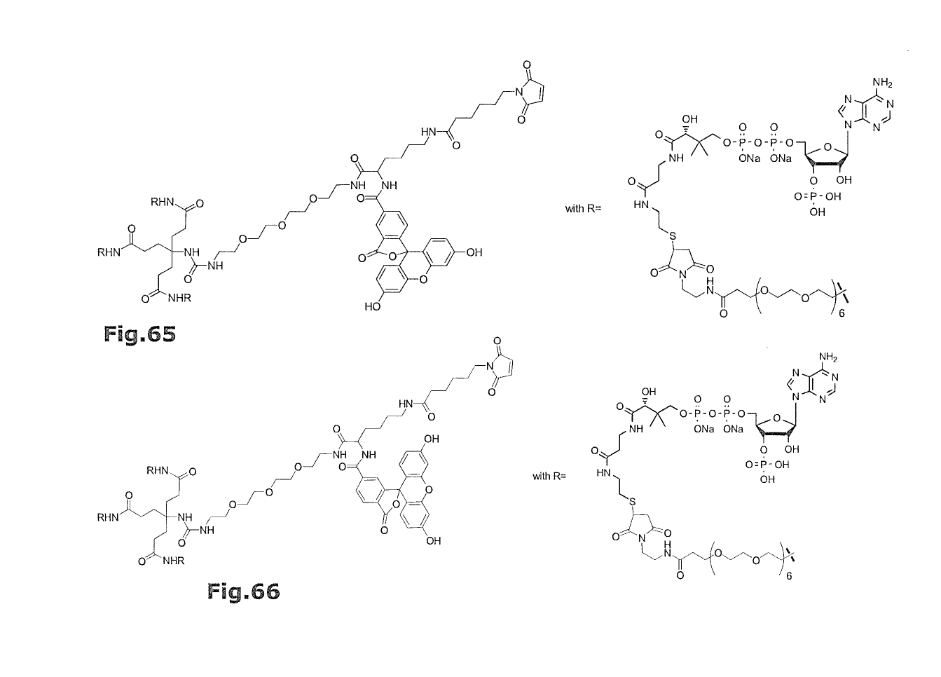

### Chemical Synthesis of N-Tris{2-(2-(2-(CoA-S-succinimido)ethylaminocarbonyl-PEG12)ethylaminocarbonyl)ethoxymethyl}methyl N′-2-(2-(2-(2-(N″-5-fluorescein-N′″-6-maleimidohexanoyl-Lys-amido)ethoxy)ethoxy)ethoxy)ethyl-urea (FIG. 65) and Corresponding 6-fluorescein Derivative (FIG. 66)

To a solution of mixture of isomers (FIG. 45) and (FIG. 46) (14 mg, 0.02 mmol) in DMF (2 mL) is added PYBOP (10 mg, 0.02 mmol) at rt. The solution is stirred at room temperature for 20 min. Then compound (FIG. 31) (100 mg, 0.02 mmol) and DIPEA (3.3 μL, 0.02 mmol) are added and the solution is heated at 50° C. for 5 min. The solution is stirred at room temperature overnight. The solvent is removed under reduced pressure, and the compound is obtained as a mixture of isomers (FIG. 65) and (FIG. 66) by purification with preparative HPLC. ESI-MS: m/z 5722.2 [M−Na]⁻.

### Example 48

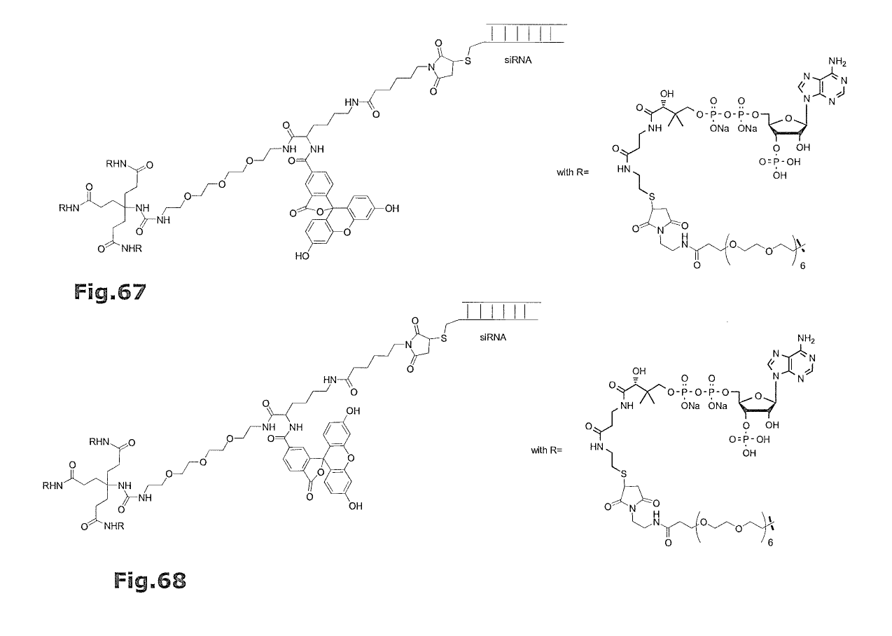

### Chemical Synthesis of N-Tris{2-(2-(2-(CoA-S-succinimido)ethylaminocarbonyl-PEG12)ethylaminocarbonyl)ethoxymethyl}methyl N′-2-(2-(2-(2-(N″-5-fluorescein-N′″-6-maleimidohexanoyl-Lys-amido)ethoxy)ethoxy)ethoxy)ethyl-urea siRNA Conjugate (FIG. 67) and Corresponding 6-fluorescein Derivative (FIG. 68)

The 5′-thiol modified oligonucleotide (43 nmol) is reduced by incubation for 1 h at room temperature with 200 mM DTT in 200 μL Tris-buffer pH 8.5. The DTT is removed by gel filtration and the oligonucleotide eluted in PBS (pH 7.4). The most concentrated fractions are combined giving a total of 800 μL. 300 μL solution of a mixture of isomers (FIG. 65) and (FIG. 66) (2.5 mM in DMF) is added and the reaction mixture incubated at room temperature for 1 h. The reaction mixture is diluted with water to a total volume of 2 mL and excess maleimide removed by gel filtration. The mixture of conjugates (FIG. 67) and (FIG. 68) is then purified by HPLC (solvent A: 0.1 M tetraethylammonium acetate pH 6.9 in water; solvent B: acetonitrile).

### Example 49

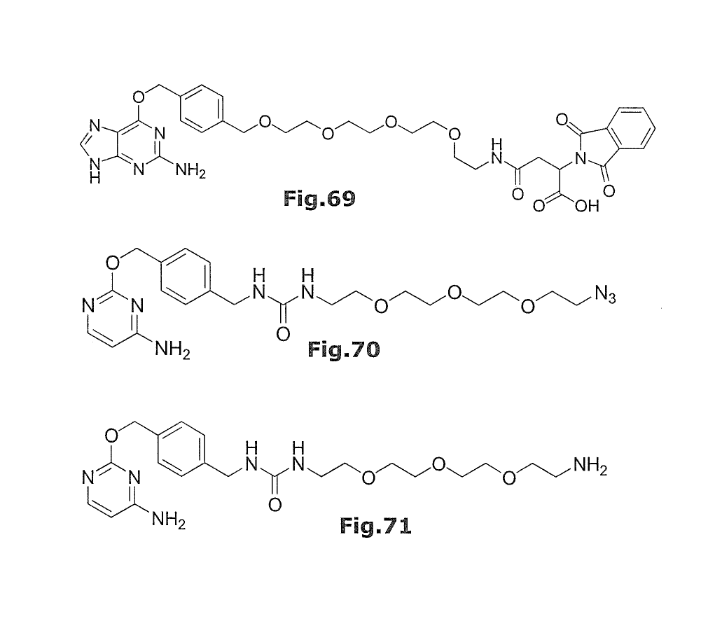

### Chemical Synthesis of 2-Phthalimido-N-(BG-PEG4)-succinic acid monoamide (FIG. 69)

To a solution of BG-PEG4-NH2 (620 mg, 1.3 mmol, 1 eq) in DMF (15 mL) is added 2-phthalimido-succinic anhydride (340 mg, 1.39 mmol, 1 eq) at rt. The reaction mixture is stirred at room temperature for 4 h, then the crude mixture is poured into water (225 mL). The pH of the water phase is adjusted to 8 with NaOH (1 M), and the precipitate disappears. The aqueous layer is washed with EtOAc (2 times 100 mL). Then the pH is adjusted to 4 and the precipitate is collected. ESI-MS: m/z 692.69 [M+H]⁺.

### Example 50

### Chemical Synthesis of N-4-((4-Aminopyrimidin-2-yloxy)methyl)benzyl-N′-2-(2-(2-(2-azidoethoxy)ethoxy)ethoxy)ethyl-urea (FIG. 70)

To a solution of 11-azido-3,6,9-trioxaundecan-1-amine (73 μL, 1 eq, 0.37 mmol) in DMF (3 mL) is added CDI (60 mg, 1 eq, 0.37 mmol). The solution is stirred overnight at rt. To the solution is added BC-NH2 (85 mg, 1 eq, 0.37 mmol) and the mixture is heated at 65° C. for 3 h. Then the crude mixture is poured onto water and extracted with ethyl acetate, the organic phase is dried over MgSO₄, and evaporated under reduced pressure to afford the desired compound. No further purification is required.

TLC(CH₂Cl₂/MeOH 10:1). ESI-MS: m/z 475.51 [M+H]⁺.

### Example 51

### Chemical Synthesis of N-4-((4-Aminopyrimidin-2-yloxy)methyl)benzyl-N′-2-(2-(2-(2-aminoethoxy)ethoxy)ethoxy)ethyl-urea (FIG. 71)

To a solution of compound (FIG. 70) (54 mg, 0.12 mmol) in dioxane (3 mL) is added water (360 μL). Then PMe₃ (720 μL 1 M in THF solution, 6 eq) is added and the solution is stirred at room temperature for 2 h. The solvent is removed under reduced pressure, and the compound (FIG. 71) is obtained by purification with preparative HPLC. ESI-MS: m/z 449.52 [M+H]⁺.

### Example 52

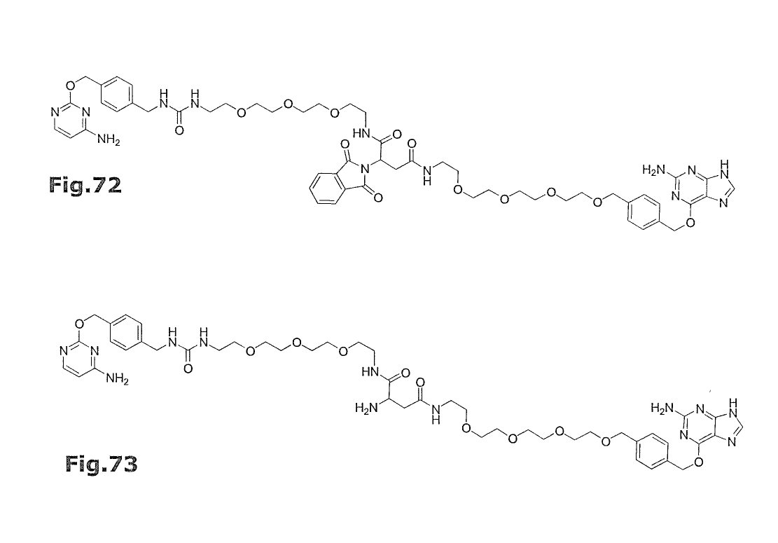

### Chemical Synthesis N-4-((4-Aminopyrimidin-2-yloxy)methyl)benzyl-N′-2-(2-(2-(2-(3-BG-PEG4-NH-carbonyl-2-phthalimido-propionylaminoethoxy)ethoxy)-ethoxy)ethyl-urea (FIG. 72)

To a solution of compound (FIG. 71) (45 mg, 0.1 mmol, 1 eq) and compound (FIG. 69) (69 mg, 0.1 mmol, 1 eq) in DMF (2 mL) are successively added DIPEA (17 μL, 0.1 mmol, 1 eq), HOBT (1 M in NMP, 0.1 mL, 0.1 mmol, 1 eq) and DCC (21 mg, 1 mmol, 1 eq) at rt. The resulting mixture is stirred overnight. Then the solvent is removed under reduced pressure and the mixture is diluted with 50 mL EtOAc. The organic layer is washed with water, dried over MgSO₄ and evaporated under reduced pressure. Flash chromatography (CH₂Cl₂/MeOH, 10/1□5/1) gives the desired compound (FIG. 72).

ESI-MS: m/z 1123.19 [M+H]⁺.

### Example 53

### Chemical Synthesis of N-4-((4-Aminopyrimidin-2-yloxy)methyl)benzyl-N′-2-(2-(2-(2-(3-BG-PEG4-NH-carbonyl-2-amino-propionylaminoethoxy)ethoxy)ethoxy)ethyl-urea (FIG. 73)

To a solution of compound (FIG. 72) (45 mg, 0.04 mmol) in EtOH (3 mL) is added methylamine (300 μl), and the solution is stirred at room temperature for 12 h. The solvent is removed under reduced pressure and the compound (FIG. 73) is obtained by purification with preparative HPLC. ESI-MS: m/z 993.09 [M+H]⁺.

### Example 54

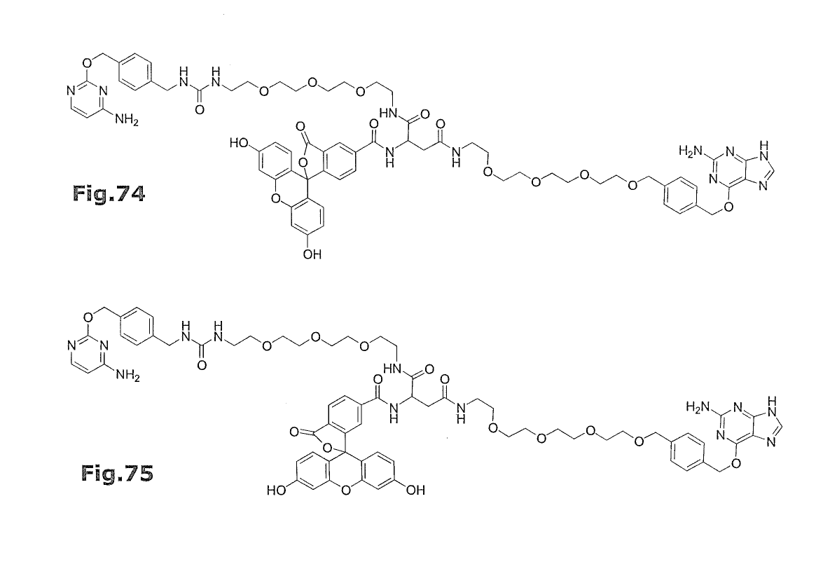

### Chemical Synthesis of N-4-((4-Aminopyrimidin-2-yloxy)methyl)benzyl-N′-2-(2-(2-(2-(3-BG-PEG4-NH-carbonyl-2-(fluorescein-5-carboxamido)propionylamino-ethoxy)ethoxy)ethoxy)ethyl-urea (FIG. 74) and Corresponding fluorescein-6-carboxamide (FIG. 75)

Compound (FIG. 73) (9 mg, 0.009 mmol) and 5(6)-carboxyfluorescein NHS ester (4 mg, 0.009 mmol) are dissolved in 800 μL DMF with Et₃N (1.35 μL, 0.009 mmol) and heated overnight at 31° C. The solvent is evaporated under vacuum and the compounds (FIG. 74) and (FIG. 75) isolated by reversed phase HPLC on a C18 column using a linear gradient of water:acetonitrile (from 95:5 to 20:80 in 20 min, 0.08% TFA).

ESI-MS: m/z 1351.39 [M+H]⁺.

### Example 55

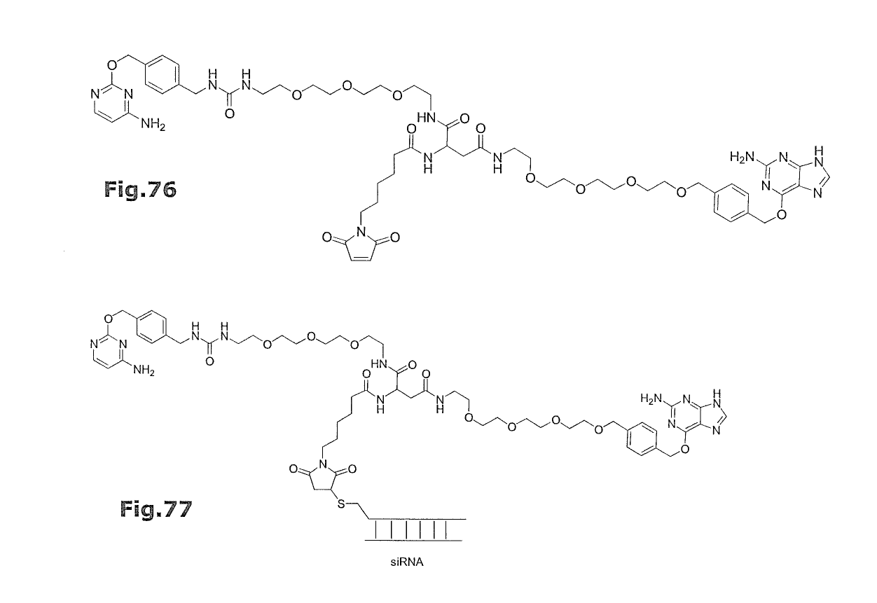

### Chemical Synthesis of N-4-((4-Aminopyrimidin-2-yloxy)methyl)benzyl-N′-2-(2-(2-(2-(3-BG-PEG4-NH-carbonyl-2-(6-maleimidohexanoylamino)propionyl-amino-ethoxy)ethoxy)ethoxy)ethyl-urea (FIG. 76)

To a solution of 6-maleimido-hexanoic acid (4.4 mg, 0.02 mmol) in DMF (1 mL) is added PYBOP (10 mg, 0.02 mmol) at rt. The solution is stirred at room temperature for 20 min. Then compound (FIG. 73) (20 mg, 0.02 mmol) and DIPEA (3.3 μL, 0.02 mmol) are added and the solution is heated at 50° C. for 5 min. The solution is stirred at room temperature overnight. Then the solvent is removed under reduced pressure and the mixture is diluted with 150 mL EtOAc. The organic layer is washed with water, dried over MgSO₄ and evaporated under reduced pressure. Flash chromatography (CH₂Cl₂/MeOH, 10/1 5/1) gives the desired compound (FIG. 76). ESI-MS: m/z 1186.29 [M+H]⁺.

### Example 56

### Chemical Synthesis N-4-((4-Aminopyrimidin-2-yloxy)methyl)benzyl-N′-2-(2-(2-(2-(3-BG-PEG4-NH-carbonyl-2-(6-maleimidohexanoylamino)propionylamino-ethoxy)ethoxy)ethoxy)ethyl-urea siRNA Conjugate (FIG. 77)

The 5′-thiol modified oligonucleotide (43 nmol) is reduced by incubation for 1 h at room temperature with 200 mM DTT in 200 μL Tris-buffer pH 8.5. The DTT is removed by gel filtration and the oligonucleotide eluted in PBS (pH 7.4). The most concentrated fractions are combined giving a total of 800 μL. 300 μL solution of compound (FIG. 76) (2.5 mM in DMF) is added and the reaction mixture incubated at room temperature for 1 h. The reaction mixture is diluted with water to a total volume of 2 mL and excess maleimide removed by gel filtration. The conjugate (FIG. 77) is then purified by HPLC (solvent A: 0.1 M tetraethylammonium acetate pH 6.9 in water; solvent B: acetonitrile).

### Example 57

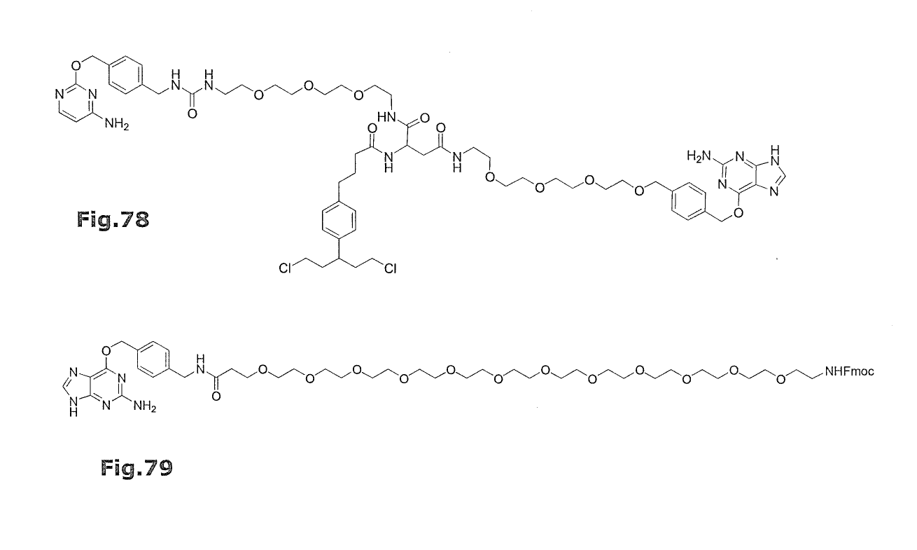

### Chemical Synthesis N-4-((4-Aminopyrimidin-2-yloxy)methyl)benzyl-N′-2-(2-(2-(2-(3-BG-PEG4-NH-carbonyl-2-chlorambucilcarboxamino-propionylaminoethoxy)ethoxy)ethoxy)ethyl-urea (FIG. 78)

To a solution of chlorambucil (6 mg, 0.02 mmol) in DMF (1 mL) is added PYBOP (10 mg, 0.02 mmol) at rt. The solution is stirred at room temperature for 20 min. Then compound (FIG. 73) (20 mg, 0.02 mmol) and DIPEA (3.3 μL, 0.02 mmol) are added and the solution is heated at 50° C. for 5 min. The solution is stirred at room temperature overnight. Then the solvent is removed under reduced pressure and the mixture is diluted with 150 mL of EtOAc. The organic layer is washed with water, dried over MgSO₄ and evaporated under reduced pressure. Flash chromatography (CH₂Cl₂/MeOH, 10/1□5/1) gives the desired compound (FIG. 78)

ESI-MS: m/z 1276.56 [M+H]⁺.

### Example 58

### Chemical Synthesis of BG-PEG12-NHFmoc (FIG. 79)

To a solution of Fmoc-amido-PEG12-acid (1 g, 1.19 mmol) in DMF (2 mL) is added PYBOP (619 mg, 1.19 mmol) at rt. The solution is stirred at room temperature for 20 min. Then O⁶-aminomethylbenzyl guanine (320 mg, 1.19 mmol) and DIPEA (196 μL, 1.19 mmol) are added and the solution is heated at 50° C. for 5 min. Then the solution is stirred at room temperature overnight. The crude mixture is poured onto diethyl ether. The precipitate is collected and washed with diethyl ether. The obtained solid is dissolved in MeOH and the solvent is concentrated until dryness. No further purification is required. MS (ESI) m/z 1093 [M+H]⁺.

### Example 59

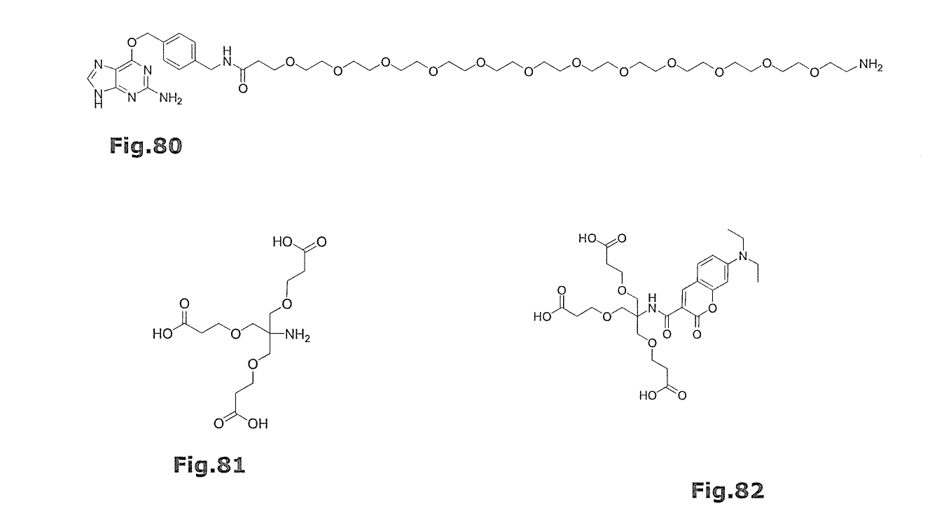

### Chemical Synthesis of BG-PEG12-NH₂ (FIG. 80)

To a solution of compound (FIG. 79) (1.5 g, 1.72 mmol) in dioxane (10 mL) is added diethylamine (2.5 mL) at rt. The solution is stirred at room temperature for 3 h. Then the solvent is removed under reduced pressure. The crude mixture is dissolved in DMF (1.5 mL) and poured into diethyl ether (10 mL). The resulting precipitate is collected. No further purification is required. MS (ESI) m/z 871 [M+H]⁺.

### Example 60

### Chemical Synthesis of Tris{[2-carboxyethoxy]methyl}methylamine (FIG. 81)

Tris-{[2-tert-butoxycarbonyl)ethoxy]methyl}methylamine (FIG. 1)(4.3 g, 8 mmol) is stirred in 80 mL of 96% formic acid for 18 h. Then the formic acid is removed at reduced pressure at 50° C. to produce a colorless oil in quantitative yield. ¹H NMR ((CD₃)₂SO, 400 MHz): 8.2 (m, 2H), 7.45 (m, 3H), 3.6 (m, 6H), 3.4 (m, 6H), 2.45 (m, 6H).

### Example 61

### Chemical Synthesis of N-Tris[(2-carboxyethoxy)methyl]methyl 7-(diethylamino)coumarin-3-carboxamide (FIG. 82)

Compound (FIG. 81) (66 mg, 0.195 mmol) and 7-(diethylamino)coumarin-3-carboxylic acid N-succinimidyl ester (70 mg, 0.195 mmol) are dissolved in 2 mL of DMF with Et₃N (28 μL, 0.195 mmol) and heated overnight at 40° C. The solvent is evaporated under vacuum and the compound (FIG. 82) isolated by reversed phase HPLC on a C18 column using a linear gradient of water:acetonitrile (from 95:5 to 20:80 in 20 min, 0.08% TFA). MS (ESI) m/z 581 [M+H]⁺.

### Example 62

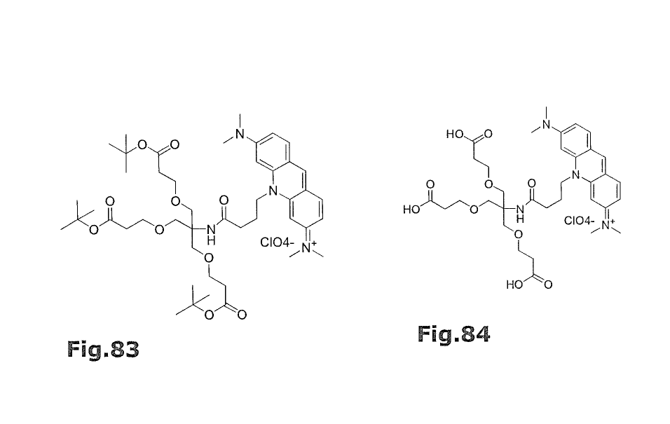

### Chemical Synthesis of N-Tris{[2-(tertbutoxycarbonyl)ethoxy]methyl}methyl ATTO-495-carboxamide (FIG. 83)

Tris{[2-(tert-butoxycarbonyl)ethoxy]methyl}methylamine (FIG. 1) (4.8 mg, 9.45 μmol) and ATTO-495 N-succinimidyl ester (5.2 mg, 9.45 μmol) are dissolved in 2 mL DMF with Et₃N (1.3 μL, 9.45 μmol) and heated overnight at 40° C. The solvent is evaporated under vacuum and the compound (FIG. 83) isolated by reversed phase HPLC on a C18 column using a linear gradient of water:acetonitrile (from 95:5 to 20:80 in 20 min, 0.08% TFA). MS (ESI) m/z 841 [M+H]⁺.

### Example 63

### Chemical Synthesis of N-Tris[(2-carboxyethoxy)methyl]methyl ATTO-495-carboxamide (FIG. 84)

Compound (FIG. 83) (210 mg, 0.25 mmol) is stirred in 250 μL of 96% formic acid for 18 h. Then the formic acid is removed at reduced pressure at 5° C. to produce a colorless oil in quantitative yield. MS (ESI) m/z 672 [M+H]⁺.

### Example 64

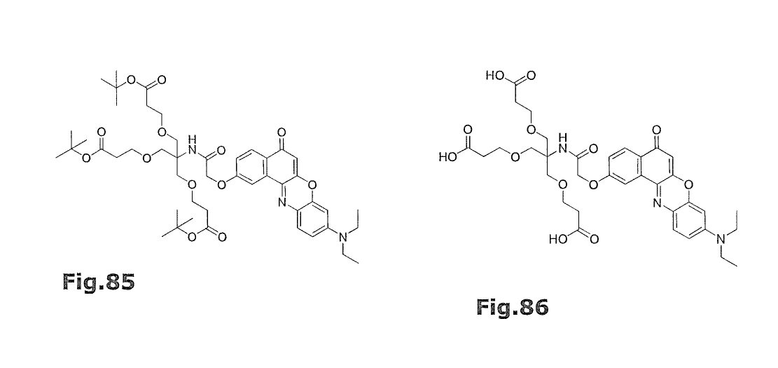

### Chemical Synthesis of N-Tris{[2-(tert-butoxycarbonyl)ethoxy]methyl}methyl nile red-oxyacetamide (FIG. 85)

To a solution of nile red-oxyacetic acid (9-diethylamino-5-oxo-benzo[a]phenoxazin-2-oxyacetic acid, 100 mg, 0.255 mmol, 1 eq) in DMF (50 mL) are successively added DCC (160 mg, 0.765 mmol, 3 eq) and NHS (90 mg, 0.765 mmol, 3 eq). The resulting mixture is stirred overnight. Then DCU salts are removed by centrifugation. Compound (FIG. 1) (130 mg, 0.255 mmol, 1 eq) and DIPEA (42 μL, 0.255 mmol, 1 eq) are added to the solution at rt. The resulting mixture is stirred overnight. Then the solvent is removed under reduce pressure. Flash chromatography (CH₂Cl₂/MeOH, 10/1□5/1) gives the desired compound (FIG. 85). MS (ESI) m/z 881 [M+H]⁺.

### Example 65

### Chemical Synthesis of N-Tris[(2-carboxyethoxy)methyl]methyl nile red-oxyacetamide (FIG. 86)

Compound (FIG. 85) (70 mg, 0.08 mmol) is stirred in 250 μL of 96% formic acid for 18 h. Then the formic acid is removed under reduced pressure at 50° C. to produce a colorless oil in quantitative yield. MS (ESI) m/z 712 [M+H]⁺.

### Example 66

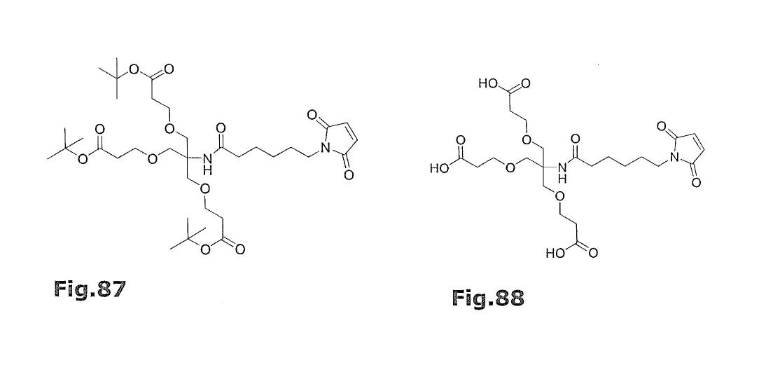

### Chemical Synthesis of N-Tris{[2-(tert-butoxycarbonyl)ethoxy]methyl}methyl 5-maleimidopentanecarboxamide (FIG. 87)

To a solution of 6-maleimido-hexanoic acid (106 mg, 0.5 mmol) in DMF (5 mL) is added PYBOP (260 mg, 0.5 mmol) at rt. The solution is stirred at room temperature for 20 min. Then compound (FIG. 1) (253 mg, 0.5 mmol) and DIPEA (83 μL, 0.5 mmol) are added and the solution is heated at 50° C. for 5 min. The solution is stirred at room temperature overnight. Then the solvent is removed under reduced pressure. Flash chromatography (cyclohexane/ethyl acetate, 1/1) gives the desired compound (FIG. 87). ¹H NMR ((CD₃)₂SO, 400 MHz): 6.7 (s, 2H), 3.7 (s, 6H), 3.65 (m, 6H), 3.5 (m, 2H), 2.45 (m, 6H), 2.1 (m, 2H), 1.6 (m, 4H), 1.45 (m, 27H), 1.35 (m, 2H).

### Example 67

### Chemical Synthesis of N-Tris[(2-carboxyethoxy)methyl]methyl 5-maleimido-pentanecarboxamide (FIG. 88)

Compound (FIG. 87) (214 mg, 0.305 mmol) is stirred in 3 mL of 96% formic acid for 18 h. Then the formic acid is removed at reduced pressure at 50° C. to produce a colorless oil in quantitative yield. The compound is directly used for next step.

### Example 68

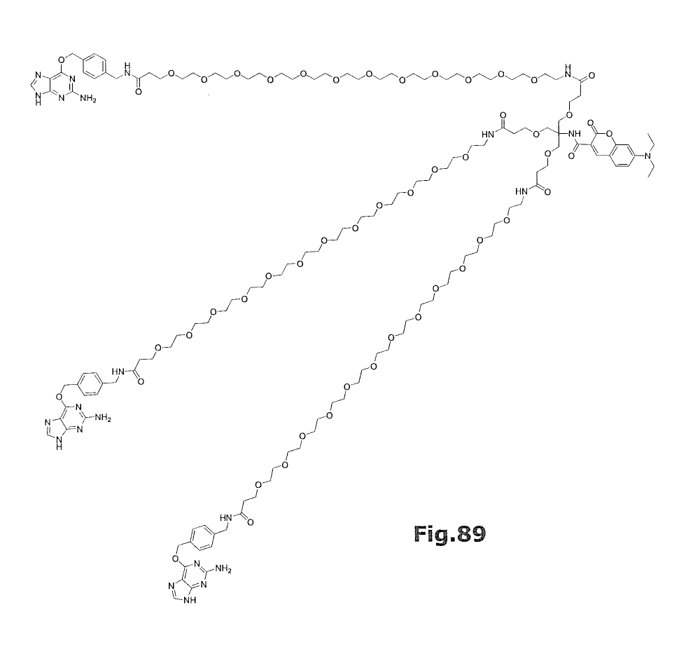

### Chemical Synthesis of N-Tris-{[2-(BG-PEG12-NH)-carbonylethoxy]methyl}methyl 7-(diethylamino)coumarin-3-carboxamide (FIG. 89)

To a solution of N-Tris[(2-carboxyethoxy)methyl]methyl 7-(diethylamino)-coumarin-3-carboxamide (FIG. 82) (10 mg, 0.018 mmol) and BG-PEG12-NH₂ (FIG. 80) (54 mg, 0.062 mmol, 3.6 eq) in DMF (1 mL) are successively added DIPEA (8 μL, 0.062 mmol, 3.6 eq), HOBT (1 M in NMP, 18 μL, 0.018 mmol, 1 eq) and EDC (12 mg, 0.062 mmol, 3.6 eq) at rt. The resulting mixture is stirred overnight. The solvent is evaporated under vacuum and the compound (FIG. 89) isolated by reversed phase HPLC on a C18 column using a linear gradient of water:acetonitrile (from 95:5 to 20:80 in 20 min, 0.08% TFA). The structural ability of compound (FIG. 89) to trigger the formation of a protein trimer is confirmed by in vitro experiments using the fusion protein SNAP-FKBP according to Example 76. The formation of the protein trimer is visualized by SDS-PAGE followed by coomassie staining of the proteins.

### Example 69

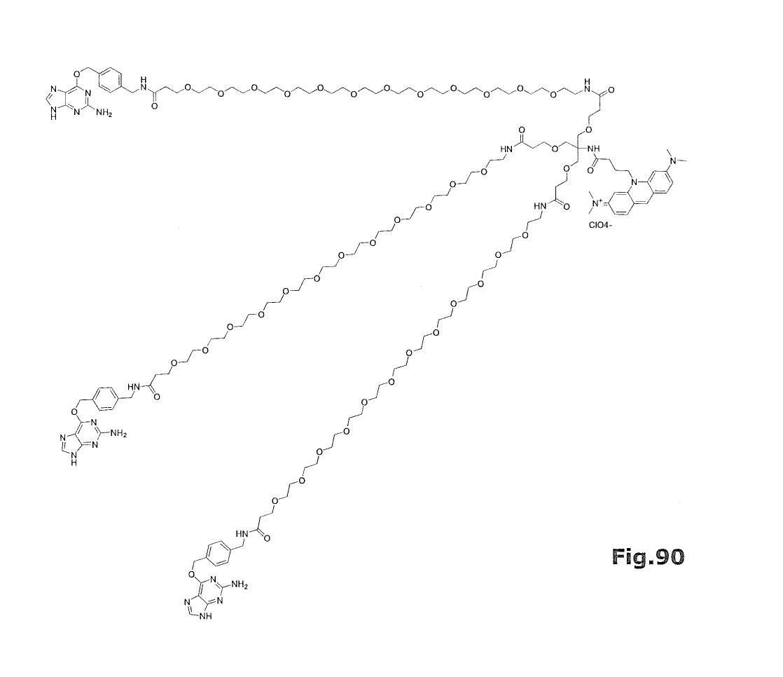

### Chemical Synthesis of N-Tris-{[2-(BG-PEG12-NH)-carbonylethoxy]methyl}-methyl ATTO-495-carboxamide (FIG. 90)

To a solution of N-Tris[(2-carboxyethoxy)methyl]methyl ATO-495-carboxamide (FIG. 84) (4 mg, 0.005 mmol) and BG-PEG12-NH₂ (FIG. 80) (15 mg, 0.0175 mmol, 3.6 eq) in DMF (1 mL) are successively added DIPEA (3 μL, 0.0175 mmol, 3.6 eq), HOBT (1 M in NMP, 5 μL, 0.005 mmol, 1 eq) and EDC (4 mg, 0.0175 mmol, 3.6 eq) at rt. The resulting mixture is stirred overnight. The solvent is evaporated under vacuum and the compound (FIG. 90) isolated by reversed phase HPLC on a C18 column using a linear gradient of water:acetonitrile (from 95:5 to 20:80 in 20 min, 0.08% TFA). The structural ability of compound (FIG. 90) to trigger the formation of a protein trimer is confirmed by in vitro experiments using the fusion protein SNAP-FKBP according to Example 76.

### Example 70

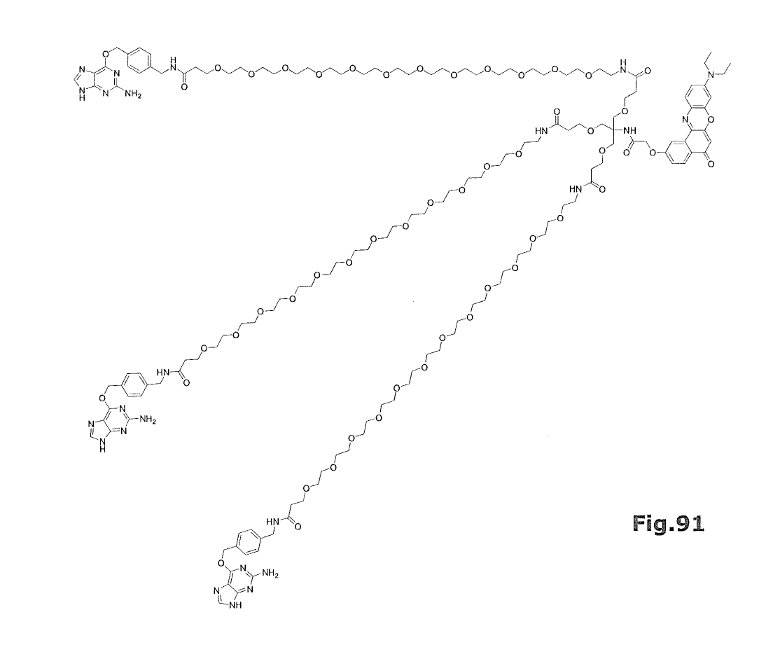

### Chemical Synthesis of N-Tris-{[2-(BG-PEG12-NH)-carbonylethoxy]methyl}-methyl nile red-oxyacetamide (FIG. 91)

To a solution of N-Tris[(2-carboxyethoxy)methyl]methyl nile red-oxyacetamide (FIG. 86) (8 mg, 0.011 mmol) and BG-PEG12-NH₂ (FIG. 80) (34 mg, 0.039 mmol, 3.6 eq) in DMF (1 mL) are successively added DIPEA (7 μL, 0.039 mmol, 3.6 eq), HOBT (1 M in NMP, 11 μL, 0.01 mmol, 1 eq) and EDC (8 mg, 0.039 mmol, 3.6 eq) at rt. The resulting mixture is stirred overnight. The solvent is evaporated under vacuum and the compound (FIG. 91) isolated by reversed phase HPLC on a C18 column using a linear gradient of water:acetonitrile (from 95:5 to 20:80 in 20 min, 0.08% TFA). The structural ability of compound (FIG. 91) to trigger the formation of a protein trimer is confirmed by in vitro experiments using the fusion protein SNAP-FKBP according to Example 76.

### Example 71

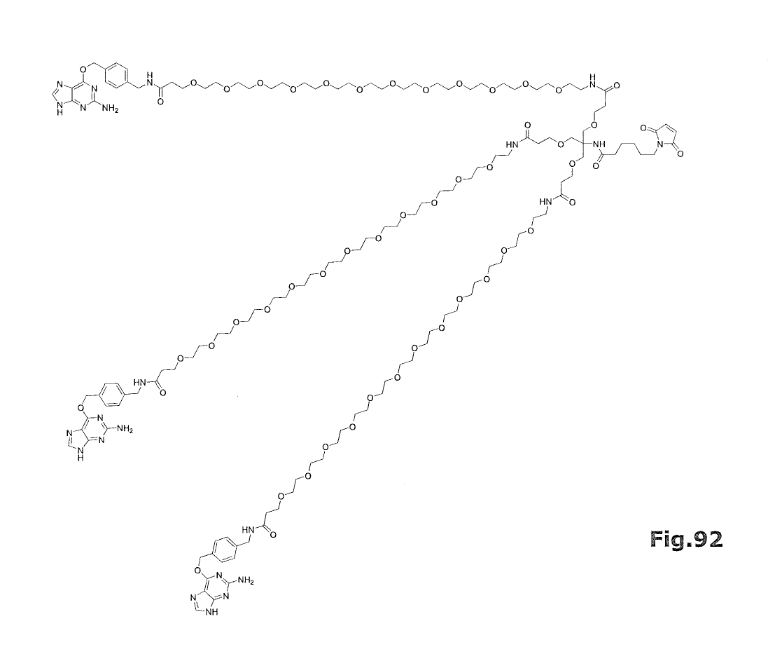

### Chemical Synthesis of N-Tris-{[2-(BG-PEG12-NH)-carbonylethoxy]methyl}-methyl 5-maleimidopentanecarboxamide (FIG. 92)

To a solution of N-Tris[(2-carboxyethoxy)methyl]methyl 5-maleimidopentanecarboxamide (FIG. 88) (8 mg, 0.016 mmol) and BG-PEG12-NH₂ (FIG. 80) (50 mg, 0.057 mmol, 3.6 eq) in DMF (1 mL) are successively added DIPEA (10 μL, 0.057 mmol, 3.6 eq), HOBT (1 M in NMP, 16 μL, 0.016 mmol, 1 eq) and EDC (2 mg, 0.057 mmol, 3.6 eq) at rt. The resulting mixture is stirred overnight. The solvent is evaporated under vacuum and the compound (FIG. 92) isolated by reversed phase HPLC on a C18 column using a linear gradient of water:acetonitrile (from 95:5 to 20:80 in 20 min, 0.08% TFA). The structural ability of compound (FIG. 92) to trigger the formation of a protein trimer is confirmed by in vitro experiments using the fusion protein SNAP-FKBP according to Example 76.

### Example 72

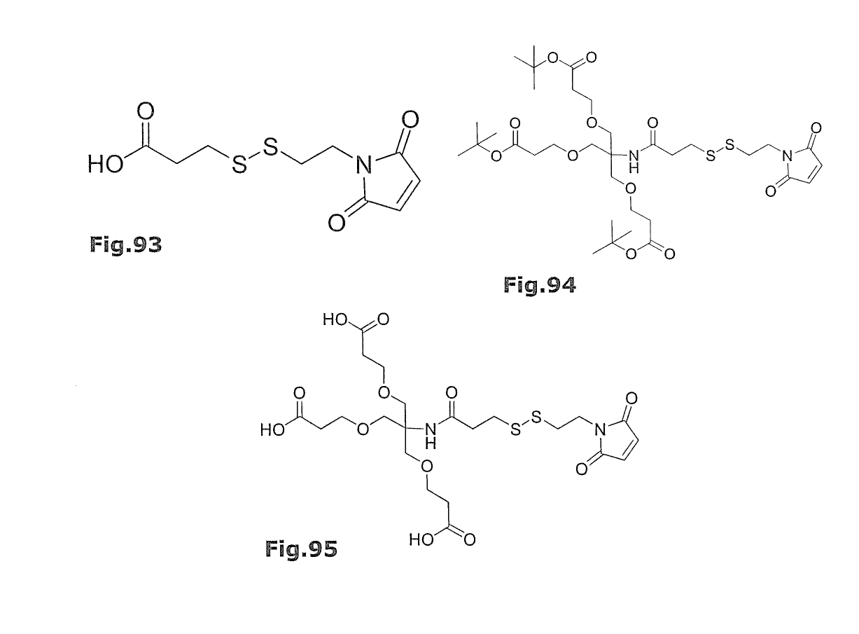

### Chemical Synthesis of 3-[2-(2-maleimidoethyl)disulfanyl]propanoic acid (FIG. 93)

A solution of 3-[2-(2-aminoethyl)disulfanyl]propanoic acid (250 mg, 1.38 mmol) and maleic anhydride (272 mg, 2.76 mmol) in a mixture of acetic acid/toluene (3/1, 3 mL) is heated overnight at 120° C. Then the crude mixture is cooled down to rt, and further cooled in an ice bath to 0° C. Pentane (50 mL) is added, and a precipitate is formed. Diethyl ether is added to this precipitate, and the white solid formed is removed. The ether solution is concentrated under vacuum to yield the product (FIG. 93). No further purification is required. ¹H NMR ((CD₃)₂SO, 400 MHz): 7.4 (s, 1H), 6.7 (s, 2H), 3.7 (m, 2H), 2.9 (m, 4H), 2.6 (m, 2H).

### Example 73

### Chemical Synthesis of N-Tris{[2-(tert-butoxycarbonyl)ethoxy]methyl}methyl 3-[2-(2-maleimidoethyl)disulfanyl]propanoylamide (FIG. 94)

To a solution of 3-[2-(2-maleimidoethyl)disulfanyl]propanoic acid (FIG. 93) (188 mg, 0.72 mmol) in DMF (2 mL) is added PYBOP (376 mg, 0.72 mmol) at rt. The solution is stirred at room temperature for 20 min. Then tris{[2-tert-butoxycarbonyl)ethoxy]methyl}methylamine (FIG. 1) (364 mg, 0.72 mmol) and DIPEA (119 μL, 0.72 mmol) are added and the solution is heated at 50° C. for 5 min.

The solution is stirred at room temperature overnight. Then the solvent is removed under reduced pressure. Flash chromatography (cyclohexane/ethyl acetate, 2/1) gives the desired compound (FIG. 94). ¹H NMR ((CD₃)₂SO, 400 MHz): 6.6 (s, 2H), 3.8 (m, 2H), 3.6 (m, 6H), 3.55 (m, 6H), 2.8 (m, 4H), 2.5 (m, 2H), 2.35 (m, 6H), 1.4 (m, 27H).

### Example 74

### Chemical Synthesis of N-Tris[(2-carboxyethoxy)methyl]methyl 3-[2-(2-male-imidoethyl)disulfanyl]propanoylamide (FIG. 95)

Compound (FIG. 94) (112 mg, 0.15 mmol) is stirred in 1.5 mL of 96% formic acid for 18 h. Then formic acid is removed at reduced pressure at 50° C. to produce a colorless oil in quantitative yield. The compound is directly used for the next step. ¹H NMR ((CD₃)₂SO, 400 MHz): 7.0 (s, 2H), 3.7 (m, 2H), 3.55 (m, 12H), 2.75 (m, 4H), 2.45 (m, 6H).

### Example 75

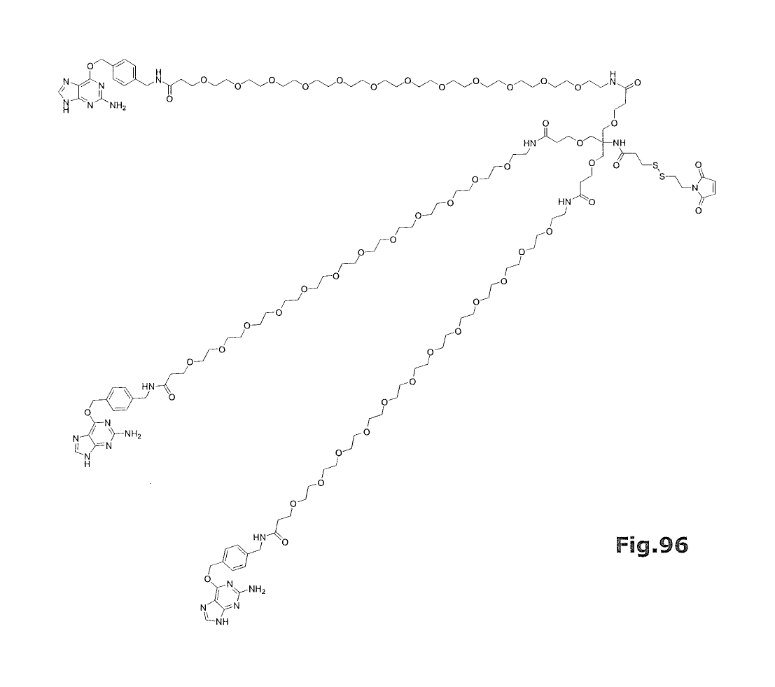

### Chemical Synthesis of N-Tris-{[2-(BG-PEG12-NH)-carbonylethoxy]methyl}-methyl-3-[2-(2-maleimidoethyl)disulfanyl]propanoylamide (FIG. 96)

To a solution of compound (FIG. 95) (10 mg, 0.017 mmol) and BG-PEG12-NH₂ (FIG. 80) (120 mg, 0.138 mmol, 8 eq) in DMF (1 mL) are successively added DIPEA (17 μL, 0.069 mmol, 4 eq), HOBT (1 M in NMP, 17 μL, 0.017 mmol, 1 eq) and EDC (14 mg, 0.069 mmol, 4 eq) at rt. The resulting mixture is stirred overnight. The solvent is evaporated under vacuum and the compound (FIG. 96) isolated by reversed phase HPLC on a C18 column using a linear gradient of water:acetonitrile (from 95:5 to 20:80 in 20 min, 0.08% TFA). The structural ability of compound (FIG. 96) to trigger the formation of a protein trimer is confirmed by in vitro experiments using the fusion protein SNAP-FKBP according to Example 76.

### Example 76

### Determination of the Reactivity of Compound (FIG. 89), (FIG. 90), (FIG. 91), (FIG. 92) and (FIG. 96) with FKBP-AGT Fusion Protein

1 μL of a 591 μM solution of FKBP protein fused to a variant of AGT available from Covalys as SNAP26™ and 1 μL of a 100 μM solution of compound (FIG. 89), (FIG. 90), (FIG. 91), (FIG. 92) or (FIG. 96) are added to 8 μL of a solution of 50 mM Tris-HCl pH 7.5; 100 mM NaCl; 0.1% Tween20™; 1 mM DTT. Following a 4 h incubation at rt, 15 μL of a solution of 100 mM Tris-HCl pH 6.8; 2% SDS; 35% glycerol; 10 mM EDTA; 20 mM DTT is added. Then the mixture is boiled for 5 min at 95° C. After cooling to rt, 25 μL of this solution is loaded on a 4-20% linear gradient SDS-PAGE gel. After electrophoresis, the proteins are coomassie stained in gel to visualize protein trimer.

### II Assembly and Expression of Components A and B

### Construction of the Expression Vectors

### Eukaryotic Expression Vectors

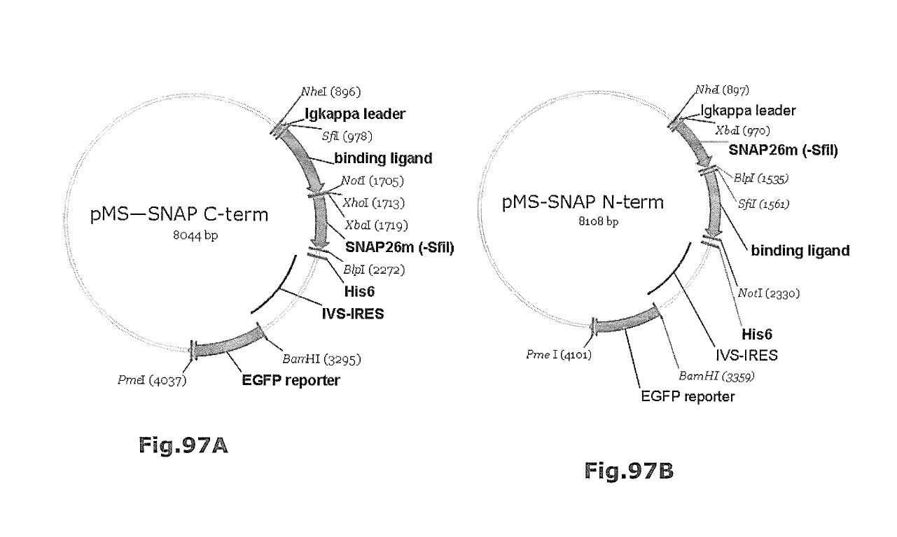

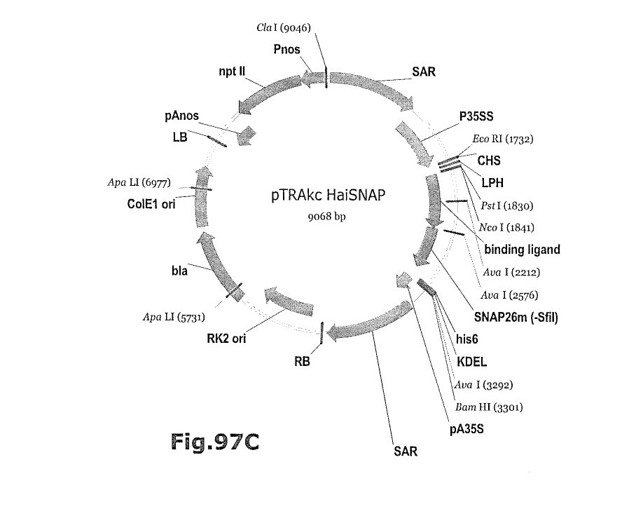

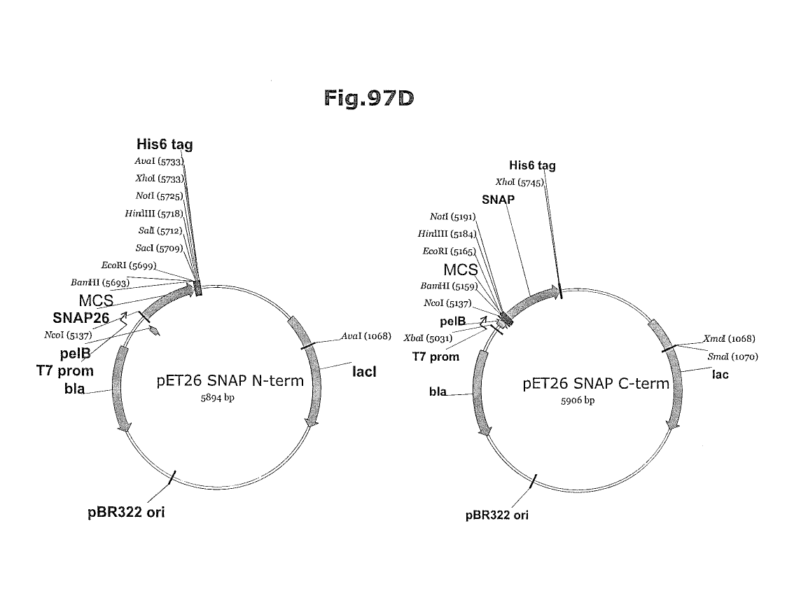

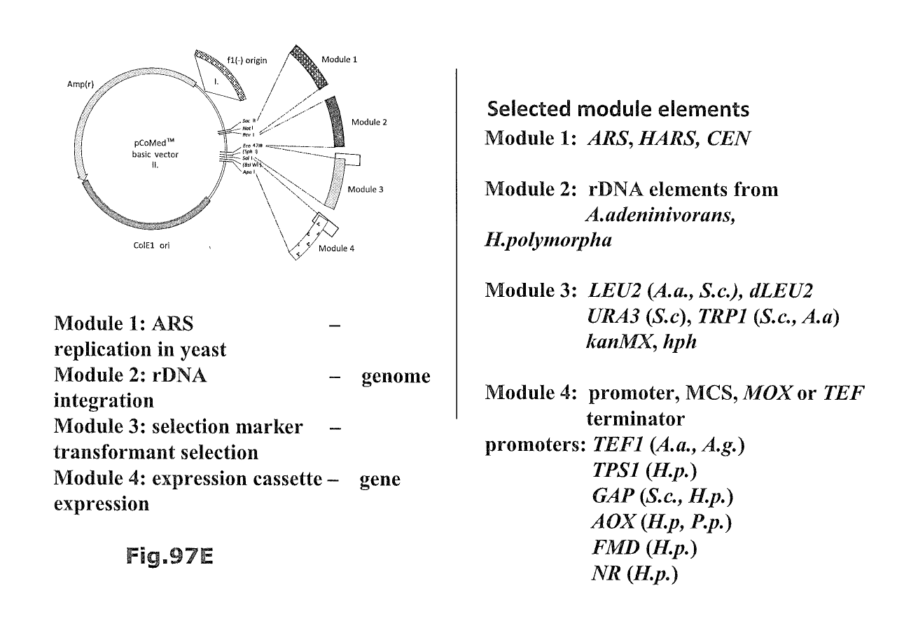

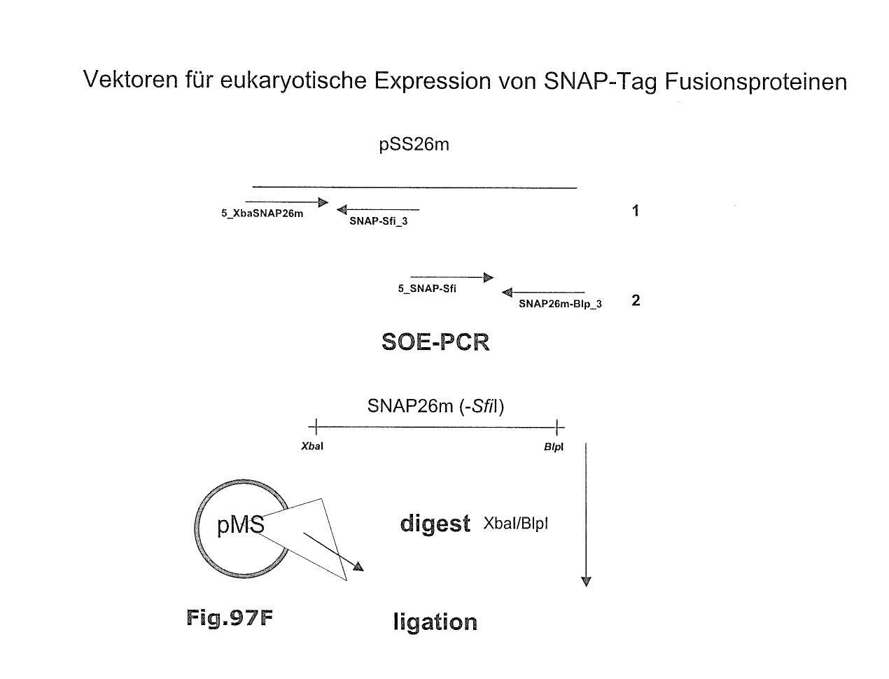

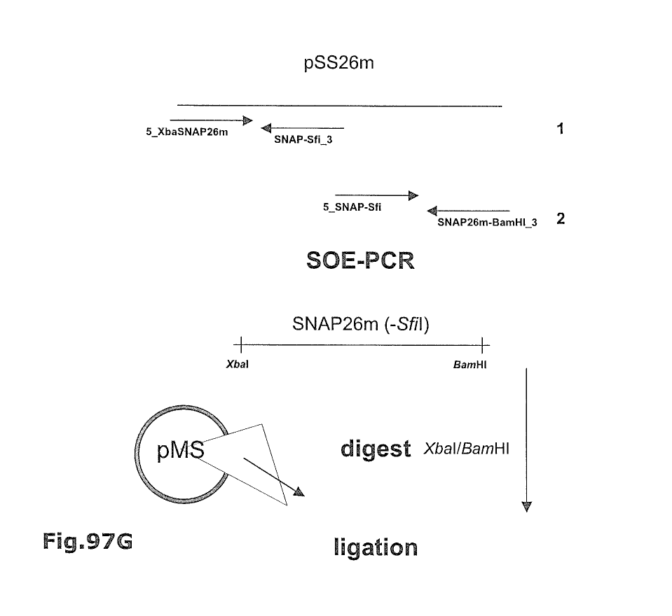

For the construction of a vector encoding a recombinant complex AB, a modified pSecTac based mammalian expression vector (pMS, Stöcker et al., 2003) was provided with the SNAP 26m gene by PCR cloning from the storage vector pSS26m (COVALYS AG). Two versions are available allowing to link component A to the N-terminus of component B or to the C-terminus of component B which are depicted in FIGS. 97A+B). In a further version of these vectors the internal SfiI endonuclease restriction site of the SNAP26m gene (Covalys) was removed to allow rapid exchange of scFv fusion partners by common SfiI/NotI cloning (FIG. 97F+G). The SfiI depleted version of the SNAP-Tag is further on named as mSNAP.

The expression cassette of the vector comprises of the following key features: the human cytomegali virus promoter sequence (CMV), a bovine growth hormone polyadenylation signal (BGH pA) and an internal IVS ribosome entry site (IRES). The SNAP-tag fusion protein is secreted through a Igkappa leader peptide whereas the reporter EGFP gene in 3′ of the IRES site is lacking a secretion signal, therefore accumulating in cytoplasm.

### Plant Expression Vectors

A plant expression vector system designed for transient and stable expression of SNAP-tag fusion proteins in plants is shown in FIG. 97C). The vector comprises the following features:

KDEL: plant ER retention signal; LPH: codon optimized murine signal peptide; Bla: ampicillin resistance (E. coli), cabenicillin resistance (A. tumefaciens); nptII: Kanamycin resistance plant; SAR: scaffold attachment region of tobacco RB7 gene; P35SS: transcription start; CHS: 5′UTR from chalcon synthase; pA35S: polyadenylation signal from CaMV; RK3 ori: ori for A. tumefaciens; ColE1 ori: ori for E. coli; LB/RB: elft/right border; pAnos: nopaline synthase polyadenylation signal; Pnos: nopaline synthase gene promoter.

### Procaryotic Expression Vectors

Procaryotic expression plasmids exemplified here are based on the pET26b™ system (Novagen) and designed for periplasmatic expression of C/N-terminal SNAP-tag fusion proteins in E. coli (FIG. 97D). The SNAP-tag version (26b) is codon optimized for and was PCR amplified from the storage vector pSET7-26b from Covalys. The expression is regulated through the T7 promoter; together with a host-encoded T7 polymerase the regulation of expression is extremely tight. The kanamycin resistance gene allows selection of transformed bacteria. The pelB leader is directing the recombinant protein into the periplasmic space.

### Yeast Expression Vectors

Yeast expression plasmid based on the CoMed™ system provided by Pharmedartis (Aachen, Germany) (FIG. 97E). The vector backbone is a derivative of a standard E. coli vector combining a ColE1 on and an ampicillin resistance (bla) sequence. A variant I contains an f1(−)origin, a variant II is without that sequence. A multiple cloning site (MCS) has been engineered for the uptake of various modules. For insertion ARS/CEN modules (module 1) are flanked by SacII/BcuI restriction sites, rDNA segments (module 2) by BcuI/Eco47III sites, selection marker modules (module 3) by Eco47III/SalI sites and expression cassettes (module 4) by SalI/ApaI sites. In variant II additional SphI and BsiWI cloning sites are present.

The Plasmid is designed to work with a variety of yeast strains:

Yeast strains (selection)

| Species | auxotrophies |
|---|---|
| Arxula adeninivorans (LS3) | wild type; leu2 |
| Arxula adeninivorans (CBS7350) | wild type; leu2 |
| Arxula adeninivorans (CBS1738) | wild type; leu2 |
| Hansenula polymorpha (CBS4732) | wild type; ura3; leu2 ura3; arg1 |
| | leu2 ura; ade1 leu2 ura3 |
| Kluyveromyces lactis | met⁻ ura3 |
| Pichia pastoris | wild type; ura3; ura3 his3 |
| Saccharomyces cerevisiae | wild type; ura3; leu2 ura3 trp1 |
| | lys2 |
| Yarrowia lipolytica E150 | wild type; ura3; leu2; ura3 leu2 |

The key features are subsegmented into four modules, whereas the modules of a concrete vector construct may contain one or more of the following features:

Module 1 consists of ARS/CEN sequences for replication in yeasts:

HARS1 (H. polymorpha-derived autonomously replication sequence)
ARS(S. cerevisiae)
CEN (S. cerevisiae)

Module 2 consists of rDNA targeting sequences for yeast genomic integration NTS2-ETS-18SrDNA-ITS1 (H. polymorpha, Arxula adeninivorans)

Module 3 consists of selection markers for transformant selection

1. dominant selection markers:

TEF promoter (A. gossypii; A. adeninivorans)—hph (E. coli)—TEF terminator (hygromycin resistance)
TEF promoter (A. gossypii; A. adeninivorans)—kanMX (E. coli)—TEF terminator (gentamycin resistance)

2. complementation selection markers:

URA3 (S. cerevisiae)
LEU2 (S. cerevisiae, A. adeninivorans)
dLEU2 (A. adeninivorans) (deficient promoter)
TRP1 (S. cerevisiae)

Module 4 comprises the SNAP-tag fusion protein expression cassette consisting of promoter—cloning site—terminator whereas the terminator sequence is mostly from MOX but also from TEF and PHO5.

### Construction of Open Reading Frames for the Component A Fused to B

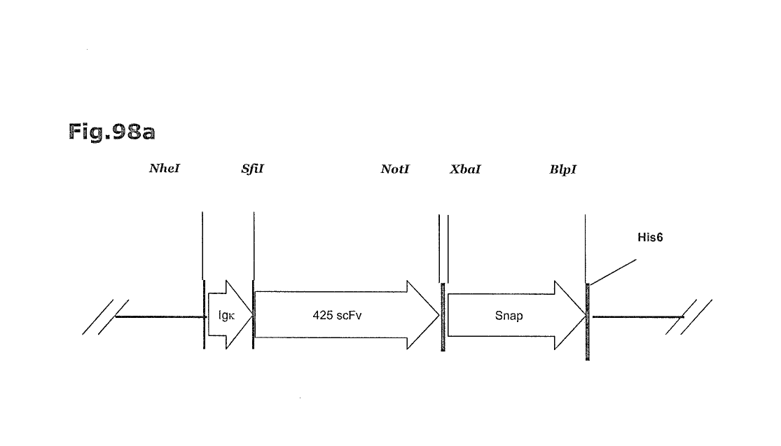

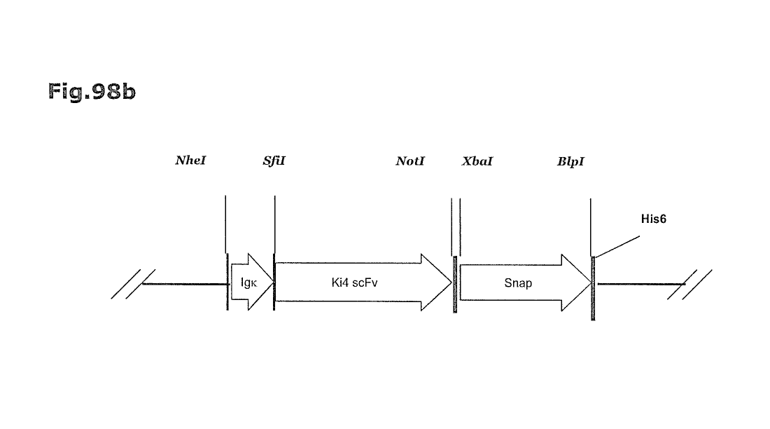

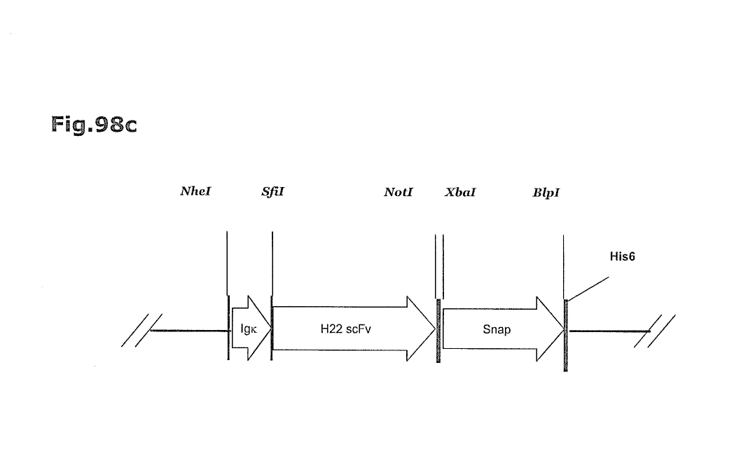

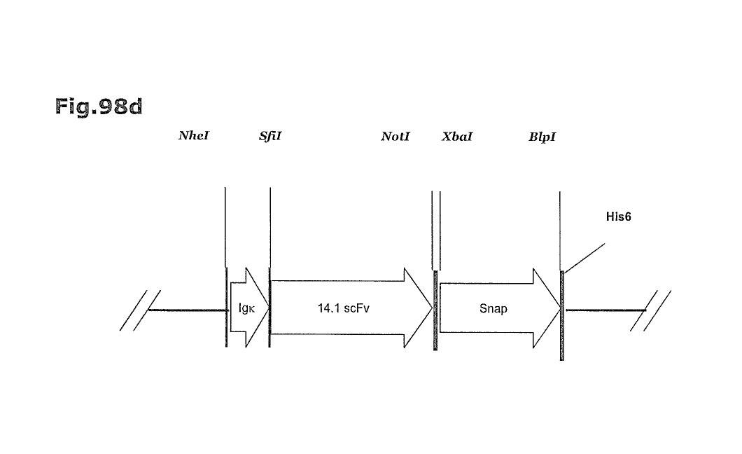

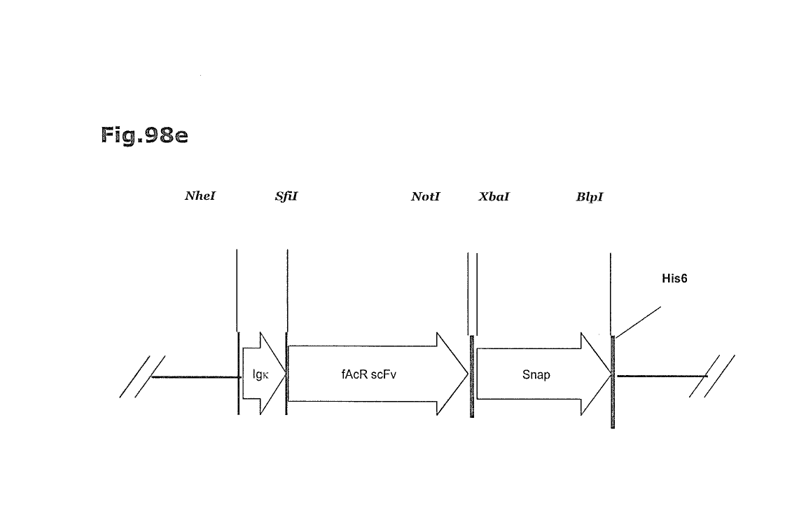

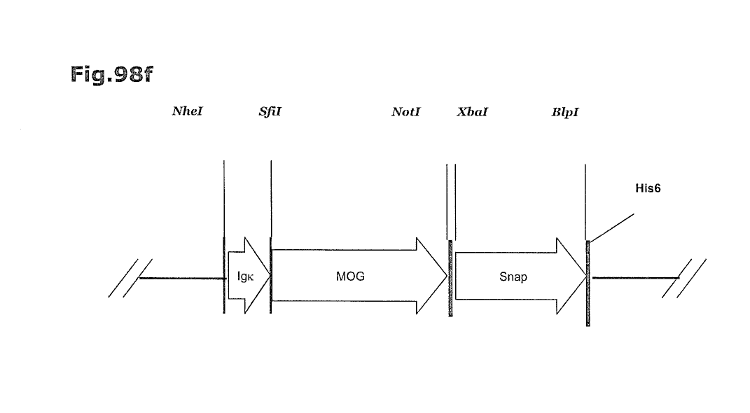

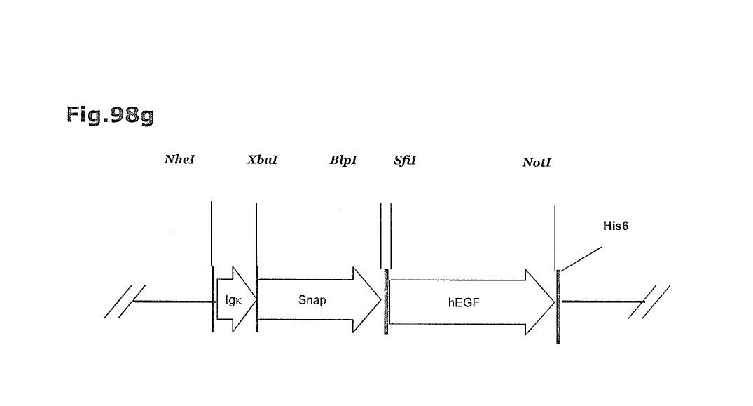

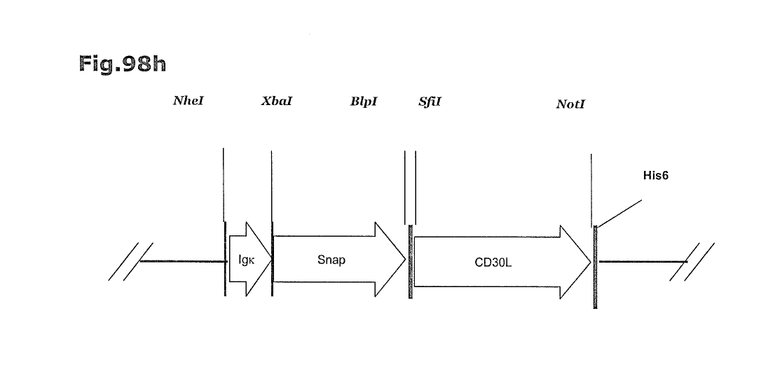

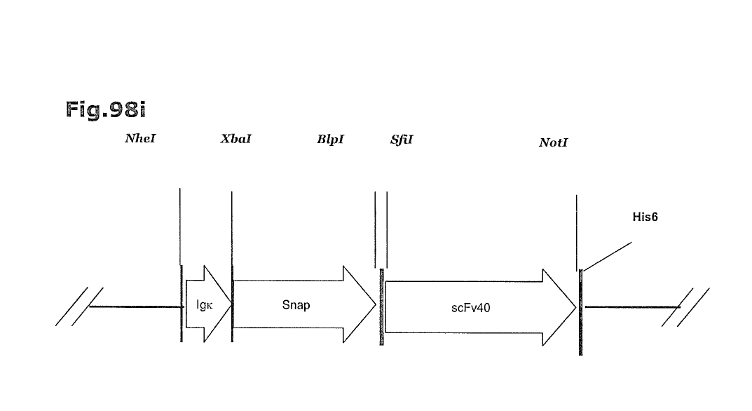

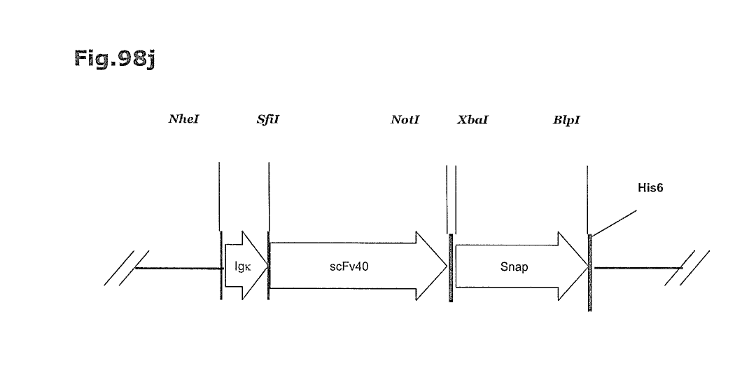

Different components A like antibody fragments and natural ligands for receptors including soluble ligands, receptors, chemokines, growth factors or interleukins or fragments thereof were cloned in an open reading frame (ORF) together with the SNAP-tag. The exemplified ORFs are listed by their sequences (expression was exemplified after cloning into the mammalian pMS vector constructs) (FIG. 98). See also list of sequences.

### Mammalian Expression of SNAP-Tag Fusion Proteins

After TransFast-mediated (Promega, Mannhein, Germany) transformation into 293T-cells, the recombinant SNAP-tag fusion proteins were expressed as described by Stocker M. et al., 2003. Briefly, one pg plasmid-DNA (like Ki4-SNAP (anti-CD30 scFv); SNAP-EGF (EGFR ligand); Hai-SNAP (anti-EGFR scFv 425); H22-SNAP (anti-CD64 scFv) or SNAP-CD30L (CD30 ligand) and 3 μl TransFast have been used according to the manufactures protocol for 12 well cell culture plates. Transfection efficiency was between 75 and 95% determined by counting green fluorescent cells. 3 days after initial transfection, cell culture supernatants were analyzed for recombinant protein. Subsequently, transfected cells were transferred into medium-sized cell culture flasks (Nunc; 85 m²) and grown in RPMI complex medium supplemented with 100 μg/ml Zeocin. One to two weeks productively transfected clones were green fluorescing and hence could be detected by fluorescence microscopy. Transfected cell populations were established by subcultivation of these clones.

### Plant Expression of SNAP-Tag Fusion Proteins

For transient expression of SNAP-tag fusion proteins in plant (e.g. tobacco-Nicotiana benthamiana), an Agrobacterium tumefaciens (A. tumefaciens) mediated transformation method is chosen.

Therefore A. tumefaciens (e.g. strain GV3101:: pMP90RK) is made electrocompetent (Shen & Forde, 1989) and 100 μl of the competent cells are mixed with 50-200 ng of a binary vector (pTRAkc based) containing the expression cassette for the SNAP-Tag fusion protein in a 0.1 cm electrogap cuvette (BioRad). The cells are transformed by a electric pulse using a GenePulser (BioRad) set at 1.8 kV, 25 mF and 200 V. Electroporated cells are incubated in 1 ml Luria-Bertani (LB) broth for 2 h prior to plating on LB medium containing 50 mg carbenicillin ml⁻¹, 50 mg rifampicin ml⁻¹ and 30 mg kanamycin ml⁻¹.

For A. tumefaciens mediated transient expression of SNAP-tag fusion proteins in tobacco plants A. tumefaciens cultures containing pTRAkc vector bearing the SNAP-tag fusion protein expression cassette clones are supplemented with 50 mg carbenicillin ml⁻¹ and 50 mg rifampicin ml⁻¹. Cultures are grown with shaking at 27° C. to exponential phase (OD600 approx. 0.8) in LB broth containing the appropriate antibiotics. Cells are collected by centrifugation at 4000 g, resuspended in induction medium (LB broth at pH 5.6 containing 10 mM MES, 20 mM acetosyringone and 2 mM MgSO₄) with the appropriate antibiotics, and grown as above. The cells are collected by centrifugation at 4000 g and resuspended in infiltration medium (10 mM MgCl2, 10 mM MES, 2% sucrose and 150 mg acetosyringone ml⁻¹, pH 5.6). The Agrobacterium suspensions are diluted in infiltration medium to an OD600 of 1.0 and are stored at 22° C. for 2-3 h.

There are two infiltration methods: direct injection and vacuum infiltration. For injection, the Agrobacterium suspensions are diluted and combined in infiltration medium, both to a final OD600 of 0.25. When Agrobacterium (pTRAkc) was co-infiltrated with the above suspension, it was used at a final OD600 of 0.0125. Leaves from 2-4-week-old Nicotiana benthamiana plants are infiltrated by injecting the bacterial suspension into the abaxial air spaces from the underside of the leaf. E.g. six leaves are agroinfiltrated with each bacterial mixture (three plants, two leaves per plant). The plants are grown for 5-6 days under conditions of 16 h light, 8 h dark, 22° C.

For vacuum infiltration, Agrobacterium cultures are grown overnight in induction medium. The cells from are resuspended in 1-8 I infiltration medium to a final OD600 of 0.25 per culture. Whole Nicotiana tabacum L. ‘Petite Havana’ SR1 plants with roots removed are submerged into the bacterial suspension and subjected to a vacuum of 290 kPa for 5-10 min, with occasional agitation to release trapped air bubbles. The vacuum is released rapidly (approx. 10 kPa s⁻¹). The plant stalks are placed in water-saturated floral foam. The plants are grown for 3 days under conditions of 16 h light, 8 h dark, 22° C.

For recombinant protein extraction N. tabacum leaf discs (cut by using the cap of a microfuge tube) are harvested from agroinfiltrated leaves and ground in 250 ml high-salt phosphate buffer (0.5 M NaCl) per disc. The extract is centrifuged at 13 000 r.p.m. for 5 min, supernatant is collected and the centrifugation is repeated.

For Western blot analysis, plant extracts were incubated at 95° C. for 2 min in loading buffer (Sambrook et al., 1989), separated by SDS-PAGE (10% gel) and then transferred onto a nitrocellulose membrane by semi-dry electroblotting. Recombinant SNAP-tag fusion protein protein is detected with anti His-tag mAb horsereadiish peroxdase coupled (1:5000). The detection reaction is done with DAB ragent (SIGMAFAST, Sigma).

The SNAP-tag fusion protein is alternatively detected in cell extracts by adding appropriate amounts of the BG stain SNAP-vista green. The manufacturers protocoll is followed regarding the staining conditions and reaction conditions. The recombinant SNAP-tag fusion proteins stained by SNAP-vista green can be visualized in a standard UV transilluminator used for gel documentation.

A scientist skilled in the art may recognize that different A. tumefaciens strains together with other binary A. tumefaciens plasmid vectors than pTRAkc may also lead to successful transformation of tobacco plants and therefore functional expression of SNAP-Tag fusion proteins.

A skilled artisan may further recognize the possibility of transformation of a variety of different hosts plants with the here described A. tumefaciens based method.

### Yeast Expression of SNAP-Tag Fusion Proteins

Yeast strains like A. adeninivorans LS3, A. adeninivorans 135, A. adeninivorans G1211 ([aleu2-), D. hansenii H158, D. polymorphus H120, P. pastoris GS115 (his4-) and the H. polymorpha MedHp1 (odc1-), as well as S. cerevisiae C13ABYS86 (MATα leu2 ura3 his pra1 prb1 prc1 cps-) are used as possible hosts (Steinborn, G. et al., 2006). All strains are grown either under non-selective conditions in complex medium (YEPD) or under selective conditions in a yeast minimal medium (YMM) supplemented with 2% of a selected carbon source (Steinborn, G. et al., 2006). Cultivation is performed at 30° C. A. adeninivorans LS3, A. adeninivorans 135, A. adeninivorans G1211, D. hansenii H158, D. polymorphus H120, H. polymorpha MedHp1, P. pastoris GS115 and S. cerevisiae C13ABYS86 are transformed according to Rösel H. et al., 1998; and Dohmen R J et al., 1991. Stable transformants are obtained after a sequence of passages on selective and non-selective media. After transformation of plasmids with the hph selection marker, hygromycin B-resistant colonies are selected on YEPD agar plates supplemented with 150-400 mg I-1 hygromycin B (200 mg I⁻¹ for A. adeninivorans LS3 and 135, 250 mg I⁻¹ for D. hansenii H158 and D. polymorphus H120, 400 mg I⁻¹ for H. polymorpha MedHp1, 150 mg I⁻¹ for P. pastoris GS115 and S. cerevisiae C13ABYS86. Single colonies are isolated and grown on YEPD medium and hygromycin B at 30° C. for 2 days. This step is repeated three times before the cells are plated on non-selective YEPD agar and grown for 3-5 days at 30° C. A single colony from each transformant is then isolated and defined as a strain.

In case of auxothrophy complementation the transformants are selected on YMM agar plates lacking the respective amino acid.

Intracellular and extracellular expression levels of SNAP-tag fusion proteins are analyzed by Western blot experiments with anti-His-Tag antibodies for the newly generated expression yeast cell lines.

For this purpose, five transformants per yeast species are cultured in YMM12% glucose at 30° C. for 72 h. The SNAP-tag fusion protein is alternatively detected in cell extracts by adding appropriate amounts of the BG stain SNAP-vista green. The manufacturers protocoll is followed regarding the staining conditions and reaction conditions. The recombinant SNAP-tag fusion proteins stained by Vista green can be visualized in a standard UV transilluminator used for gel documentation.

### Bacterial Expression of SNAP-Tag Fusion Proteins

For bacterial expression of SNAP-tag fusion proteins the desired fusion partners are cloned into the pET26b+ derived bacterial periplasmic expression vectors described in “construction of expression vectors”.

Heat shock competent bacteria of the appropriate E. coli strain (e.g. ROSETTA, EMD Biosciences, Darmstadt, Germany) are transformed by e.g. heat shock transformation. Seleced clones growing on agar plates with Kanamycin (pET encoded Kan^{R} provides bacteria with resistance gene) are taken for expression.

The expression of the plasmid encoded SNAP-Tag fusion proteins is done using the osmotic stress expression protocol described in Barth et al., 2000.

Recombinant RFT5-SNAP-tag fusion proteins are expressed under the control of the IPTG inducible T7 lac promoter in E. coli ROSETTA (DE3). Bacteria are grown overnight at 26° C. in Terrific Broth (TB) (Sambrook&Maniatis, 1989) containing 50 mg of kanamycin/ml and 0.5 mM ZnCl2, since it has been shown earlier that periplasmic proteolysis can be dramatically reduced upon addition of this salt (Baneyx, F., and G. Georgiou. 1992.). The shaking culture is diluted 30-fold in 200 ml of the same medium. At an optical density at 600 nm (OD600) of 2, it is supplemented with 0.5 M sorbitol, 4% NaCl, and 10 mM glycine betaine and is then incubated at 26° C. for additional 30 to 60 min. Thereafter, SNAP-tag fusion protein production is induced by the addition of 2 mM IPTG at 26° C.

Fifteen hours later, cells are harvested by centrifugation at 3,700 3 g for 10 minat 4° C. For all the following steps, tubes are chilled on ice. The bacterial pellet is centrifuged, and its wet weight is determined. Cells are frozen at −80° C. until further processing.

The expression and purification of RFT5-SNAP by IMAC is performed as described in section “IMAC purification of SNAP-Tag fusion proteins fromm mammalian expression”.

IMAC Purification of SNAP-Tag Fusion Proteins from Mammalian Expression

Purifications of the His-tagged proteins were accomplished by the Ni-NTA metal-affinity method (Hochuli, V., 1989, Porath, J. et al., 1975). The protein purification followed a modified protocol for the purification of native protein from Qiagen (The Expressionist 07/97). For protein mini-preparation, 900 μl centrifugation-cleared cell culture supernatant was supplemented with 300 μl of 4× incubation buffer (200 mM NaH₂PO₄, pH 8.0; 1.2 M NaCl; 40 mM Imidazol) and 30 μl 50% Ni-NTA. Following 1 h incubation, the Ni-NTA resin was pelleted by centrifugation. After washing the sediment twice in 175 μl 1× incubation buffer, bound protein was eluted with 30 μl of elution buffer (50 mM NaH₂PO₄, pH 8.0; 1.2 M NaCl; and 40 mM imidazol) and 30 μl 50% Ni-NTA. Following an 1 h incubation, the Ni-NTA resin was pelleted by centrifugation. After washing the sediment twice in 175 μl 1× incubation buffer, bound protein was eluted with 30 μl of elution buffer (50 mM NaH₂PO₄, pH 8.0; 300 mM NaCl; 250 mM Imidazol) for 20 min at RT. Larger scale purification of eukaryotically-expressed proteins up to 500 ml cell culture supernatant was performed on a AEKTA FPLC system (Amersham-Pharmacia, USA). Cell culture supernatants were loaded onto a Ni-NTA column and following elution of the His-tagged proteins were made under the conditions described above.

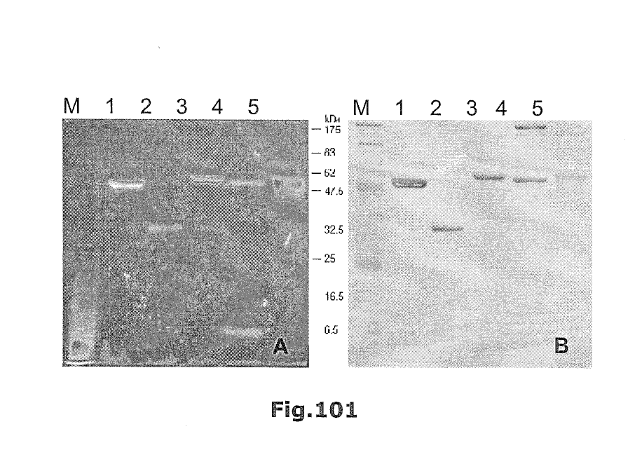

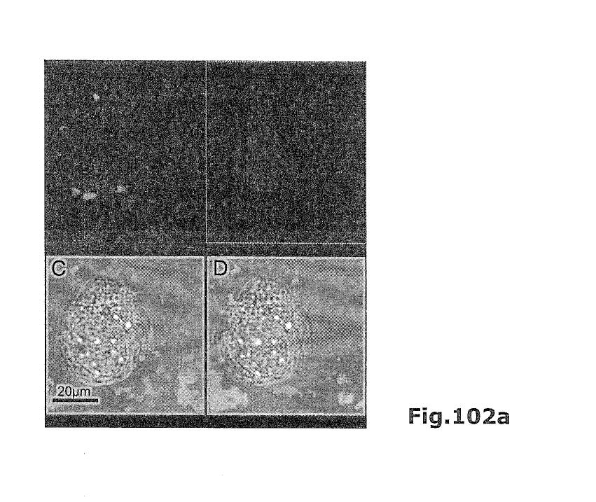

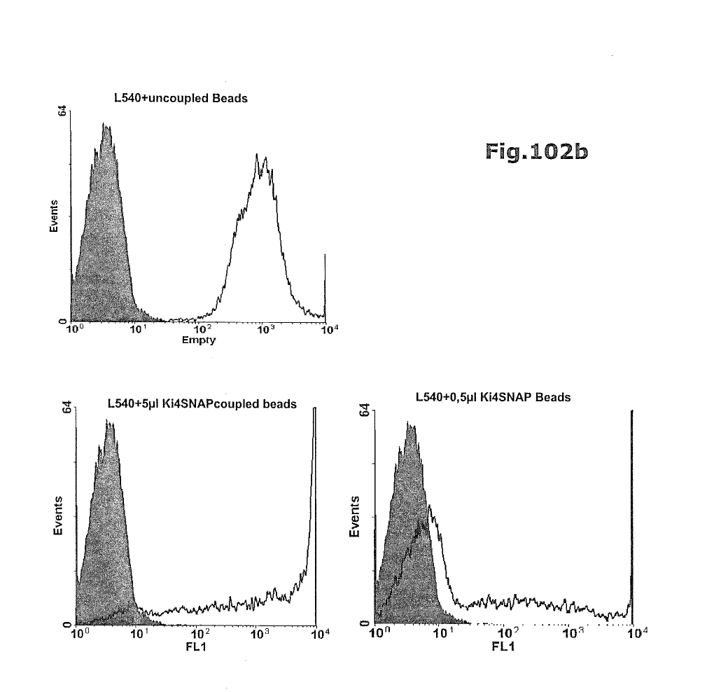

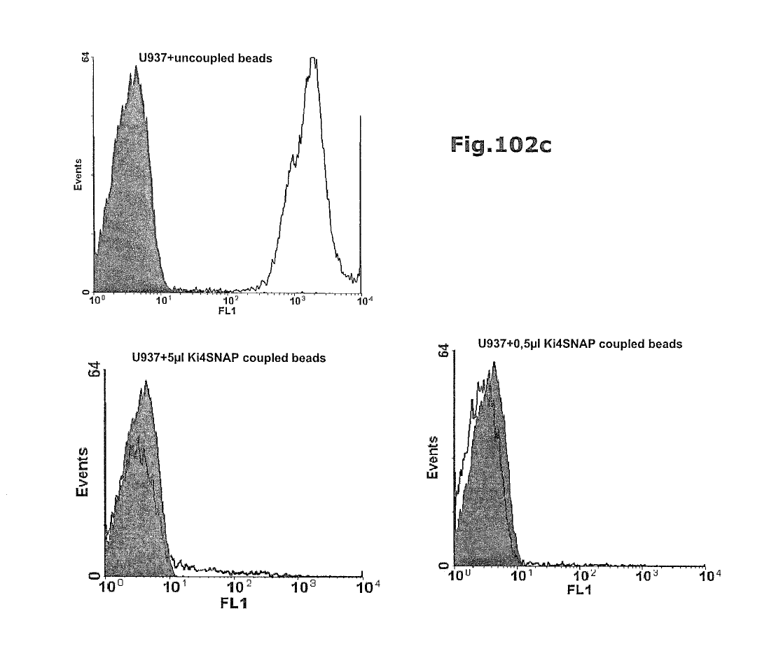

FIG. 101) shows a 12% SDS-PAGE gel (A: UV light, B: Coomassie stained) which was loaded with 5 μg of the different mammalian expressed and IMAC (Immobilized Metal Affinity Chromatography) purified. The Gel contains: 1:Ki4-SNAP; 2: SNAP-EGF; 3: Hai-SNAP; 4: H22-SNAP; 5: SNAP-CD30L; M: prestained portein marker (NEB).

### III Complex ABC and its Use

### Labeling of SNAP-Tag Fusion Proteins with BG Derivatives of Organic Fluorophores

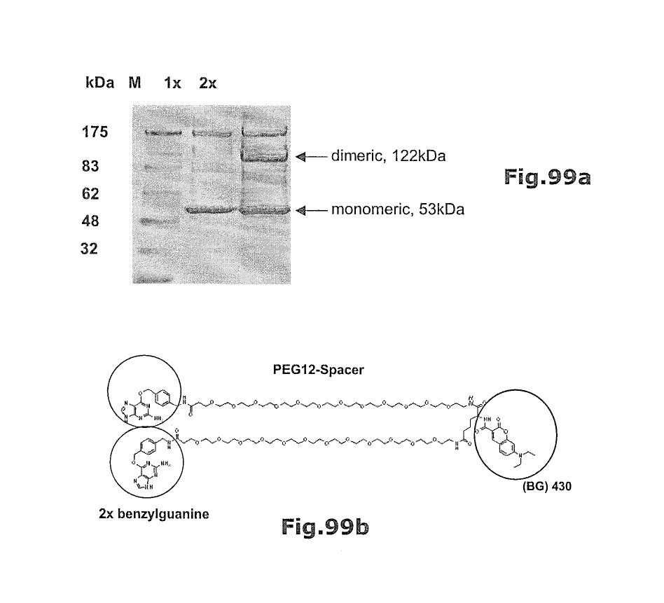

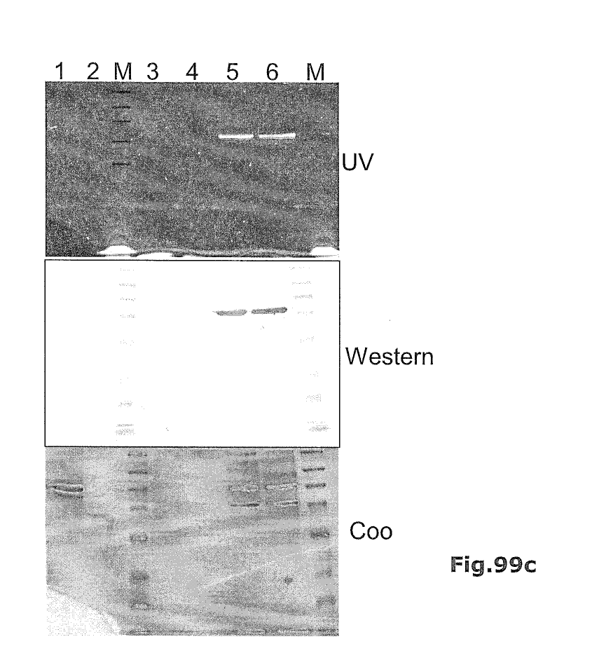

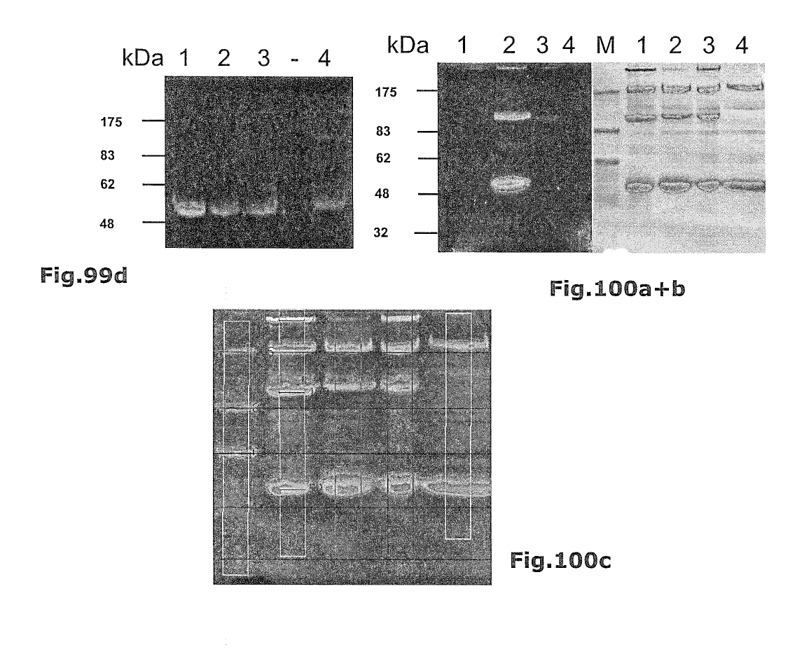

In a first step the SNAP-tag fusion protein (Ki4-SNAP) is Ni-NTA purified as described in section “IMAC purification of SNAP-Tag fusion proteins from mammalian expression”. While still bound on the resin via His-Tag-Nickel interaction the Ki4-SNAP protein can be labeled with one of the SNAP-tag specific BG substrates like e.g BG505 as seen in FIG. 99c).

A labeling solution of BG-505 2 μM is prepared in 1× Ni-NTA wash buffer (300 mM NaCl, 50 mM sodium phosphate, pH=7.5). As much solution as the estimated void volume of the Ni-NTA resin is prepared and added to the column. The incubation is done at room temperature for 30 minutes in the dark. The resin is washed twice with 5 bed volumes of Ni-NTA wash buffer. The elution of CT-fluorophor labeled His-tagged protein is done with a Ni-NTA elution buffer (300 mM NaCl, 50 mM sodium phosphate, 500 mM immidazole, pH=7.5).

The success of the labeling reaction is documented by SDS-PAGE followed by analysis under a UV transilluminator (BioRad Gel Doc XR gel documentation) (FIG. 99c).

Furthermore the labeling success is documented with a Intas CRI-Maestro In vivo imager.

Labeling of CLIP-Tag Fusion Proteins with CT Derivatives

CLIP-tag fusion proteins are Ni-NTA purified as described for SNAP-tag constructs. While still bound on the resin via His-Tag-Nickel interaction the CLIP-Tag fusion proteins can be labeled with one of the CLIP-tag specific CT substrates like CT-360,CT-430, CT-FL/CT-PF, CT-488, CT-505, CT547, CT-TMR, CT-647 CT-Biotin, CLIP-vista Green.

A labeling solution of CT-505 2 μM is prepared in 1× Ni-NTA wash buffer (300 mM NaCl, 50 mM sodium phosphate, pH=7.5). As much solution as the estimated void volume of the Ni-NTA resin is prepared and added to the column. The incubation is done at room temperature for 30 minutes in the dark. The resin is washed twice with 5 bed volumes of Ni-NTA wash buffer. The elution of CT-fluorophor labeled His-tagged protein is done with a Ni-NTA elution buffer (300 mM NaCl, 50 mM sodium phosphate, 500 mM immidazole, pH=7.5).

The success of the labeling reaction is documented by SDS-PAGE followed by analysis under a UV transilluminator (BioRad Gel Doc XR gel documentation). Furthermore the labeling success is documented with a Intas CRI-Maestro In vivo imager.

Labeling of ACP-/MCP-Tag Fusion Proteins with CoA-Derivatives in Living Cells

Wash the ACP-Tag-Eotaxin/MCP-Tag-CXCL9 expressing HEK293 cells three times with tissue culture medium with serum. One vial of ACP-tag substrate is dissolved in 25 μL of DMSO to give a labeling stock solution of 1 mM in DMSO. After 10 minutes of mixing all the ACP-tag substrate is dissolved.

The 1 mM ACP-tag substrate stock solution is diluted 1:200 in medium to give a labeling medium of 5 μM. Afterwards MgCl2 to a final concentration of 10 mM is supplemented. Finally, the ACP-Synthase is added to a final concentration of 1 μM.

The culture medium on the cells expressing an ACP-tag fusion protein located in or on the cell membrane with the ACP-tag facing the outside of the cell is exchanged with the labeling medium and incubated for 30 minutes. Afterwards the labeling medium is removed and exchanged by fresh cell culture medium and incubated for another 20 minutes to remove unreacted ACP-tag substrate. The medium is exchanged again and the cells are ready for microscopy, flow cytometric analysis or FACS sorting.

The same procedure can be operated with cells expressing a MCP-Tag fusion protein. Therefore the labeling substrate is the same, a CoA derivative but instead of the ACP-Synthase the SFP-Synthase is taken for catalyzing the labeling reaction.

Labeling of Purified ACP-/MCP-Tag Fusion Proteins with CoA Derivatives

ACP/MCP-tag fusion proteins are Ni-NTA purified as described for SNAP-Tag constructs. While still bound on the resin via His-Tag-Nickel interaction the ACP/MCP-Tag fusion proteins can be labeled with one of the ACP/MCP-tag specific CoA based substrates like CoA-488, CoA-547, CoA-647 and CoA-Biotin.

One vial of ACP-tag substrate is dissolved in 25 μL of DMSO to give a labeling stock solution of 1 mM in DMSO. After 10 minutes of mixing all the ACP-tag substrate is dissolved.

As much solution as the estimated void volume of the Ni-NTA resin is prepared and added to the column. The incubation is done at room temperature for 30 minutes in the dark. The resin is washed twice with 5 bed volumes of Ni-NTA wash buffer. The elution of BG-fluorophor labeled His-tagged protein is done with a Ni-NTA elution buffer (300 mM NaCl, 50 mM sodium phosphate, 500 mM immidazole, pH=7.5).

The same procedure can be performed with cells expressing a MCP-Tag fusion protein. Therefore the labeling substrate is the same, a CoA derivative but instead of the ACP-Synthase the SFP-Synthse is taken for catalyzing the labeling reaction.

The success of the labeling reaction is documented by SDS-PAGE followed by analysis under a UV transilluminator (BioRad Gel Doc XR gel documentation). Furthermore the labeling success is documented with a Intas CRI-Maestro In vivo imager.

### Homo-/Hetero Bivalent Antibody-SNAP-Tag Conjugates

The moldular structure of the invention related complex allows the combination of two SNAP-tag constructs with (antibody) fusion partners of different/same binding specificity via a linker structure containing two or more BG residues, resulting in a bispecific molecule.

For the construction of heterobivalent (bispecific) constructs the whole process consists of two steps to maximize the amount of built heterodimers.

In a first step a recombinant SNAP-tag fusion protein with specificity 1 was bound on the resin via His-Tag-Nickel interaction.

A solution of 2 μM of the desired homobifunctional BG-crosslinker (FIG. 3b) was prepared in 1× Ni-NTA wash buffer (300 mM NaCl, 50 mM sodium phosphate, pH=7.5). As much solution as the estimated void volume of the Ni-NTA resin was prepared and added to the column. The incubation was done at room temperature for 30 minutes in the dark. The resin was washed twice with 5 bed volumes of Ni-NTA wash bufferto remove unreacted crosslinker.

The elution of BG-crosslinker labeled His-tagged protein was done with a Ni-NTA elution buffer (300 mM NaCl, 50 mM sodium phosphate, 500 mM immidazole, pH=7.5).

In a second step the recombinant SNAP-tag fusion protein with specificity 2 was added in the same molar ratio than the prelabeled protein 1. The crosslinking reaction was then performed at 4° C. over night in solution.

The success of the crosslinking reaction was documented by SDS-PAGE and Coomassie staining (FIG. 99a).The gel shows the Ki4-SNAP (lane lx) and its crosslinked version (lane 2×) together with a molecular weight marker (lane M). The molecular sizes determined by densitometric analysis are given as 53 kDa for the single Ki4-SNAP and 122 kDa for the crosslinked version. Crosslinking was realized with a homobifunctional crosslinker, SV 305, containing a PEG 12 spacer (FIG. 99b). Crosslinkers like in FIG. 99b) comprising a fluorophor were additionaly documented with a CRI-Maestro In vivo Imager (INTAS, Göttingen, Germany) which is able to excite and detect all kinds of fluorophors from 430 nm up to 800 nm. It is able to assess emissions wavelengths from 500 up to 900 nm.

Successfully coupled SNAP-tag fusion proteins were detctable down to amounts of 50 ng depending on the quantum yield of the fluorophor.

### Bi-/Multimeric SNAP-Tag Fusion Proteins Conjugates

Analogous to the in “bispecific antibody-SNAP-tag conjugates” described procedure di- or multimeric complexes with one binding specificity can be produced by crosslinkers having two or more BG moieties.

The reaction can also be done IMAC matrix assisted like for bispecifics but also without in a single step reaction.

For a single step reaction the IMAC purified SNAP-tag fusion protein is mixed with the crosslinker in the following ratio: for a crosslinker with a given number of BG residues BGₙ the mixture formula is:

(n)Mol SNAP fusion+1M BGₙ

The reaction mix is incubated for at least 12 h at 4° C. in the dark.

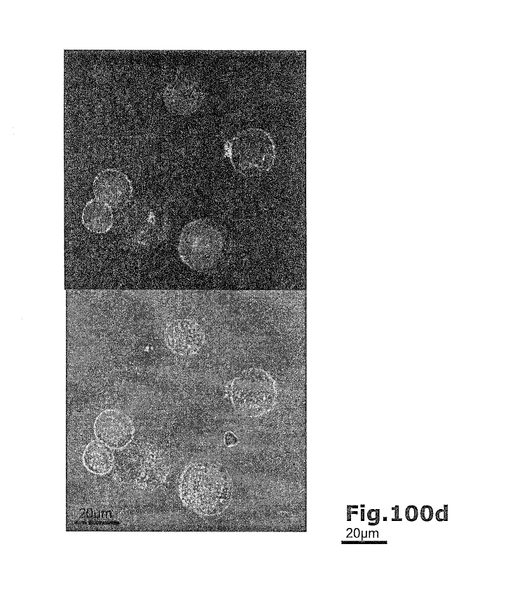

FIGS. (100a-d) shows: (FIG. 100a): composite picture of Hai-SNAP fusion protein labelled with three different fluorophor labeled homotrimeric crosslinkers and visualized by Cri-MAESTRO In vivo Imager. (FIG. 100b): the same gel coomassie stained and (FIG. 100c): the same coomassie stained gel analyzed with a densitometric analysis software. The HaiSNAP was mixed with the crosslinkers in a molar ratio of 3:1. The fluorophors can be well detected using implemented conventional emission filter sets. Samples 1: C1776-4 labelled with BG430 (Ex 421 nm, Em 444 nm and 484 nm), 2: C1884-4 labelled with Atto 495 (Ex: 495, Em:527); 3: C1883-4 labelled with nile red (ex.: 554 nm; ex: 638). For chemical structures see FIGS. 15,16 and 17. The chemical structure formulas are depicted in FIGS. (89-91).

FIG. (100d) shows a confocal microscopy done with the SV305 crosslinked version of Hai-SNAP. The crosslinked protein was separated from non crosslinked version by 100 kDA MWCO spin columns (Pall Nanosep). In brief 25 μg of the crosslinked sample were added to the column and cenrifuged at 10.000 g for 10 minutes. Afterwards 500 μl 1×PBS were added and the sample centrifuged again until the volume was reduced to 50 μl. This step was repeated and the residue in the column was taken for microscopic analysis.

The staining of 5×105 L3.6 μl cells was done as described in section “Confocal microscopy applications of SNAP-tag fusion proteins”.

### Antibody-Nucleic Acid Conjugates (RNA)

Optimized siRNAs were synthesised by Dharmacon with an amino-group and C(6) spacer on the 3′ or 5′ end of the sense strand. The siRNA duplexes are solubilised in PBS and reacted with a 50fold molar excess of BG-GLA-NHS (Covalys) solubilized in water free DMF for 4 h at RT. In the next step the siRNA is ethanol precipitated and residual BG-GLA-NHS is removed by passing through a gel filtration column (Centri-spin 10, Princeton separations). Analogously thiol-modified RNA can be used together with a BG-maleimide after reduction of the thiol with DTT. FIG. (104A) shows to schematic procedure for si-RNA coupling to SNAP-tag fusion proteins.

The results of a coupling reactions of anti eEFII siRNA to H22-SNAP and to Hai-SNAP were separated on a 10% SDS-PAGE. The gel shows the following samples: 1: H22-SNAP+a eEFII-BG; 2: H22-SNAP; 3: Hai-SNAP+a eEFII-BG and 4: Hai-SNAP. FIG. (104B) is an ethidiumbromide stained gel analyzed under a standard UV transilluminator (BioRad). FIG. (104C) is the same gel subsequently coomassie stained. The siRNA coupled SNAP-tag fusion proteins show a clear electromobility shift in comparison to their uncoupled versions. The siRNA coupled complexes run as exprected around 15 kDa. higher in size (65 kDa instead of 50 kDa).

### Antibody-Nucleic Acid Conjugates (DNA)

For targeted delivery of DNA molecules the DNA is modified with Benzylguanine either by direct modifications of oligonucleotides with a terminal benzylguanine (BG) or benzylcytosine (BC). For longer DNA stretches or whole plasmids the DNA fragment is amplified by PCR using a BG or BC modified oligonucleotide as one of the two primers.

The PCR product is purified from unreacted BG or BC modified oligonucleotides via a commercial plasmid preparation kit (EndoFree Plasmid Maxi Kit QIAGEN, Hilden Germany).

The purified PCR product is then incubated with the SNAP-tag fusion protein in a molar ratio of (RNA:SNAP-tag fusion protein) 2:1 over night at 4° C. The success of the coupling reaction is monitored via agarose gel electrophoresis followed by ethidiumbromide staining where only the DNA labeled SNAP-tag fusion proteins are stained whereas the SNAP-tag fusion protein alone is not stained. A discrimination between DNA labeled and non labeled fusion proteins is also possible via the electromobility shift of labeled protein.

The successful DNA labeled Ki4-SNAP-tag fusion protein is able to target cancer cells via their overexpressed cell surface marker CD30.

After binding of the protein-DNA complex it is internalized via receptor mediated endocytosis processes. An alternative route of internalization is the electroporation of cells after binding of the complex with a nucleofector (AMAXA).

In another embodiment the SNAP-tag fusion protein-DNA complexes are at first coupled via specific DNA-DNA interaction on a DNA loaded surface. After coupling the complexes are able to fix CD30 overexpressing cells on certain spots, where the Protein-DNA complex was immobilized beforehand.

### Directed Immobilization of SNAP-Tag Fusion Proteins on Particles

In this certain embodiment silica nanobeads with a size distribution between 20 and 80 nm and encapsulated rhodamine fluorophor were taken. The beads were concentrated at 9.5 mg/ml with 5.3×10¹⁵ amino (NH₂) groups (8.76×10⁻³ μmol/ml).

500 μl beads (containing 4.38 nmol NH² groups) were pelleted with 1500 g for 2 min, washed 2× with dry DMF and resuspended in 80 μl DMF.

Beads in DMF were added to an excess of BG-GLA-NHS (415 nmol [symbol] 95fold molar excess) and incubated 1 h at 25° C. with shaking.
Beads were washed twice with 800 μl PBS, resuspended with 200 ml Ki4-SNAP (100 μg, 2 nmol) in PBS/1 mM DTE and incubated for one hour at room temperature. Beads were washed two times with 800 μl PBS and resuspended in 100 μl PBS prior to use.

FIG. (102A) shows a confocal microscopy of Ki4-SNAP functionalized Nanobeads binding CD30-positive L540 cells. Rhodamine based emission of beads in red (A) and Draq5 emission in blue pseudocolour (B), overlay (D) with grayscale picture (C).

FIG. (102B) shows the flow cytometric analysis of cD30 overexpressing L540cy cells incubated with different amounts (0.5 and 5 μl) of Ki4-SNAP coupled rhodamine doted nanobeads. As control 5 μl uncoupled beads were applied to L540cy cells.

FIG. (102C) shows the flow cytometric analysis of the CD30 negative U937 cells incubated with different amounts (0.5 and 50) of Ki4-SNAP coupled rhodamine doted nanobeads. As control 5 μl uncoupled beads were applied to the U937 cells.

Direct ELISA with SNAP-Tag Fusion Proteins

A 96 well ELISA plate is coated with the analyte by pipetting 25 μl of coating buffer (100 mM Sodiumcarbonate, pH 9.6) in each well and then mixing with 25 μl analyte solution per well.

After 2 hours of coating at room temperature the plate is washed twice with 1×PBS and then 50 μl of the detection antibody solution (SNAP-tag fusion protein, 50-100 ng) is added. The SNAP-tag fusion protein was labeled beforehand with the SNAP-vista green fluorophor (Covalys) as described in “Labeling of SNP-tag fusion proteins with BG derivatives of organic fluorophores”. The detection antibody solution is incubated for 1 hour at room temperature and the plate is washed twice with 1×PBS. Afterwards the plate is analyzed in a fluorescence ELISA reader using s filter set suited for the SNAP-tag coupled fluorophor.

Sandwich ELISA with SNAP-Tag Fusion Proteins

ELISA plate surfaces can be modified with BG-PEG-NH2 so that they will covalently immobilize SNAP-tag fusion proteins. Surface activation is done using standard amino-coupling procedures (such as exposure to NHS and EDC). This surface is then modified as follows: 1.4 mg (0.0031 mmol) BG-PEG-NH2 is dissolved in 10 mL HBSbuffer and centrifuged (20,000×g, room temperature, 20 min). 100 μL of this solution is pipetted into each well of a 96 well ELISA plate with carboxylated surfaces. After 30 minutes of incubation excess reactive groups are quenched by adding ethanolamin (10 mmol) and further 10 minutes incubation. After three times of washing with 1×PBS the ELISA plate surface is ready to use for the direct immobilization of SNAP-tag fusion protein from samples. Therefore 100 ng of purified Ki2 mab (in 50 μl PBS) are pipetted into each well and incubated for 2 hours at room temperature or alternatively over night at 4° C.

After washing two times with 1×PBS the sample (500) containing the analyte (secreted CD30) is pipetted into the wells and incubated for 2 hours at room temperature. The Plate is then washed twice with 1×PBS before 50 μl of the detection antibody solution (scFv Ki3, 200 ng/well) is applied. The detection antibody consists either of a scFv-GFP fusion or a fluorophore labeled scFv-SNAP-tag fusion protein for fluorescent readout.

### Immuno-PCR

This protocol is basically a modified version of the quantitative immuno-PCR (qIPCR) method described by Niemeyer et al., 2007.

The basic principle of the assay relies upon a sandwich immunoassay followed by qPCR 1:capture of antigen by a non labeled antibody (Ki2 mab) coated on the 96 well PCR/ELISA plate and 2: sCD30 antigen capture from blood serum and 3: the detection of this captured sCD30 by a fusion protein of Ki3 scFv and SNAP-tag which was beforehand covalently labeled with a dsDNA PCR template and finaly 4: a qPCR step for signal amplification and readout.

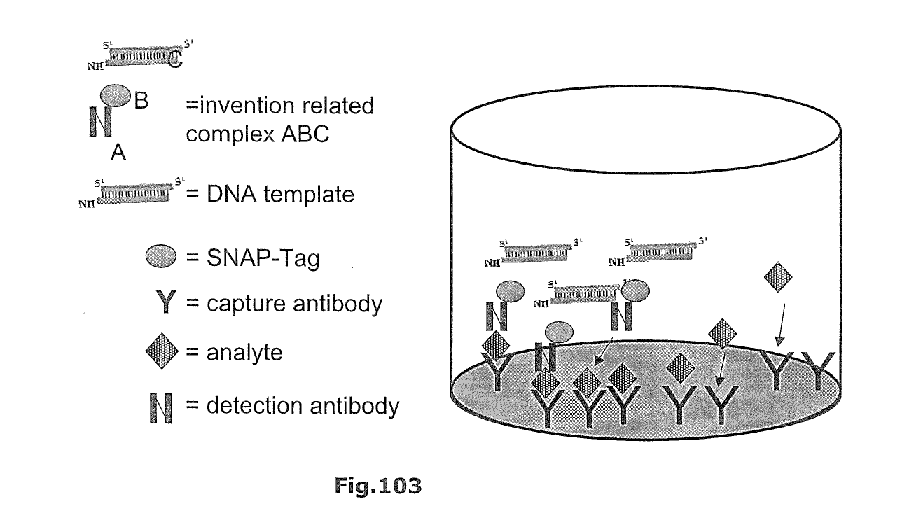

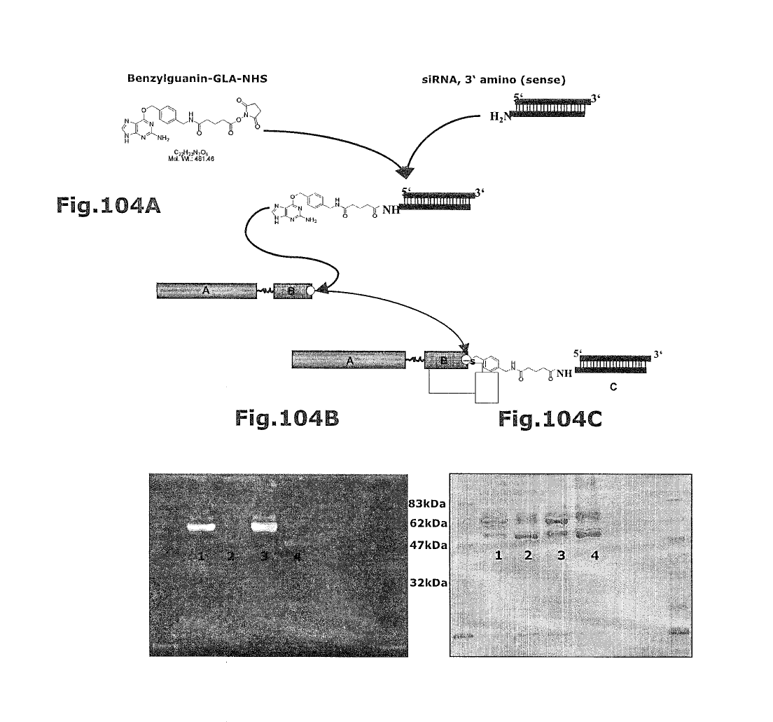

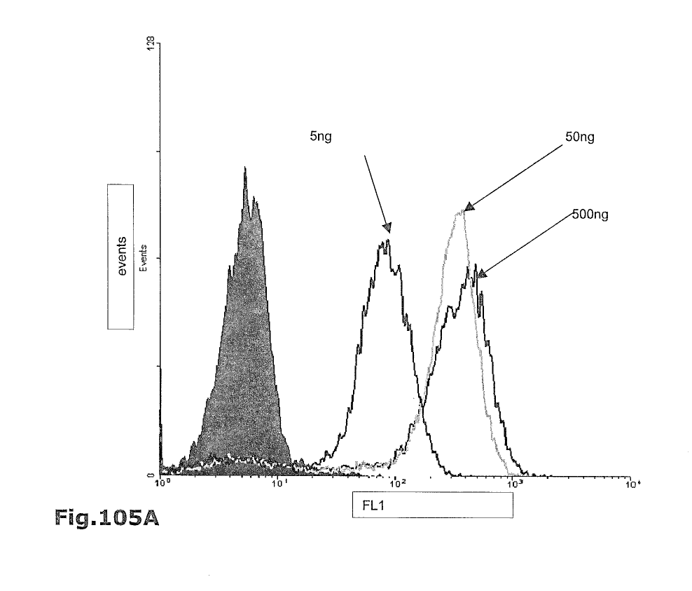

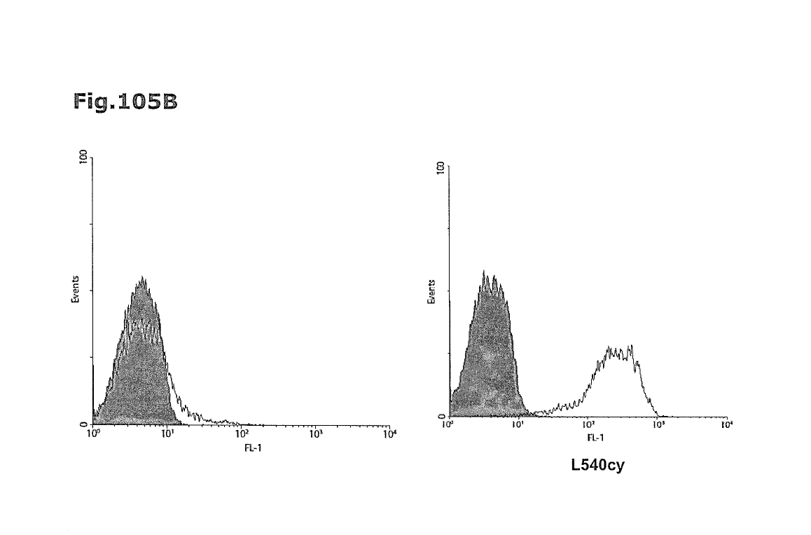

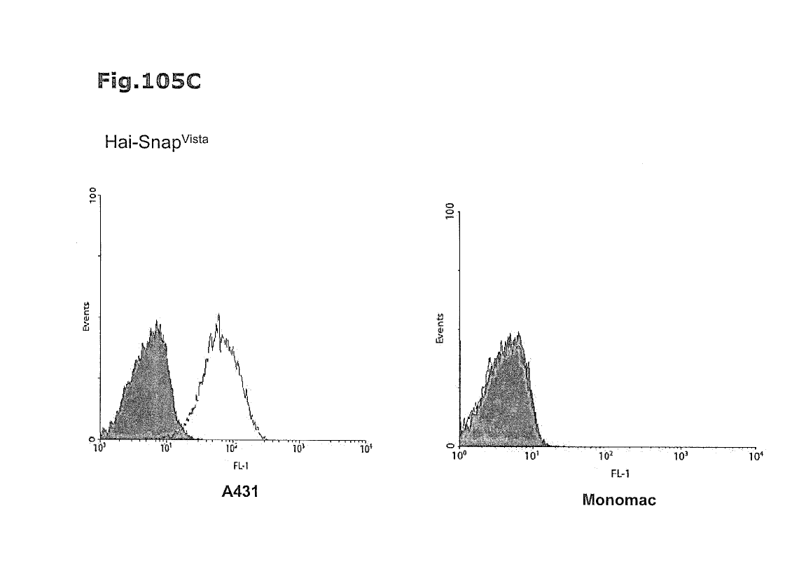

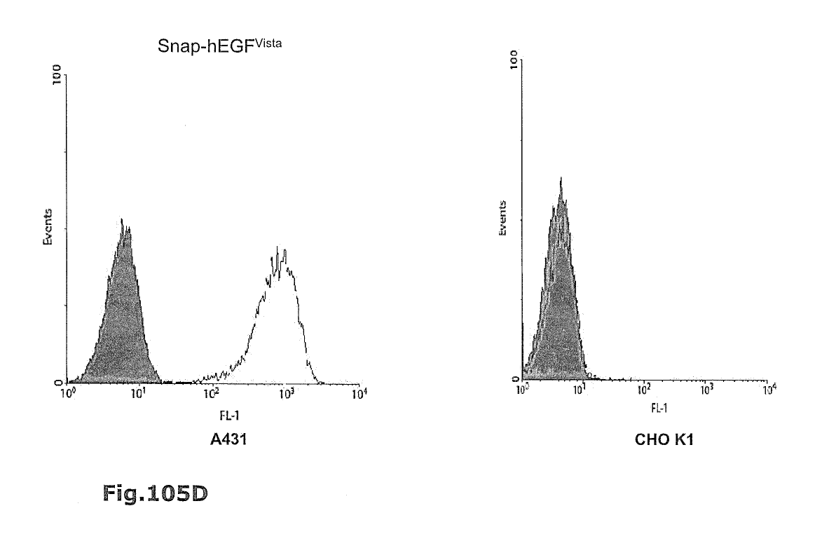

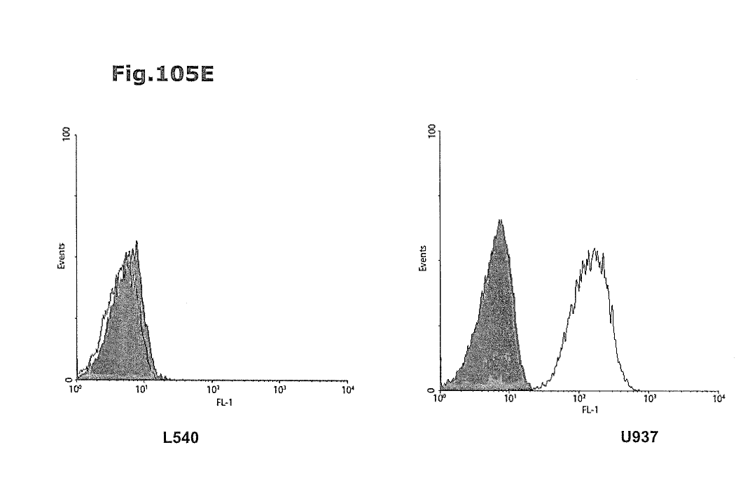

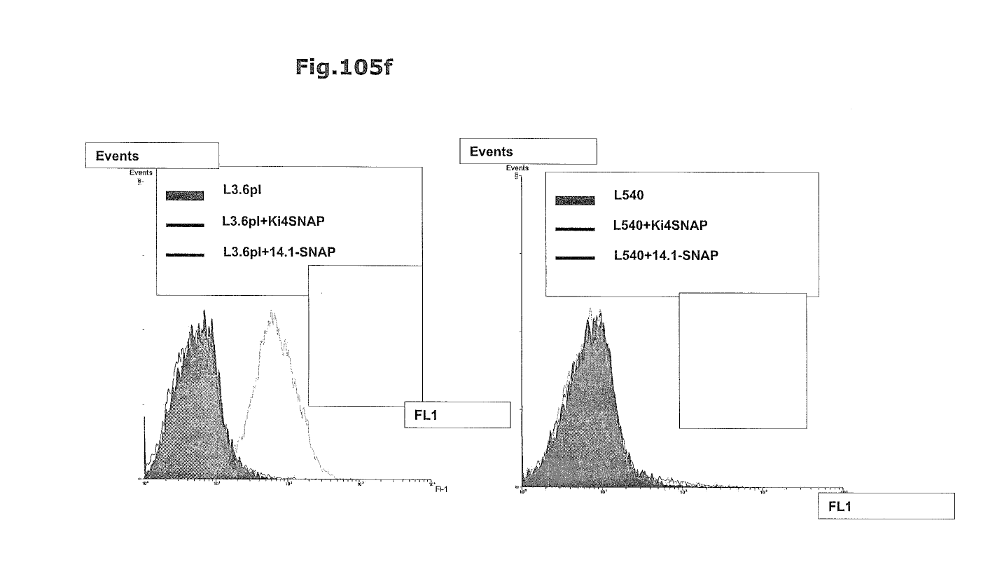

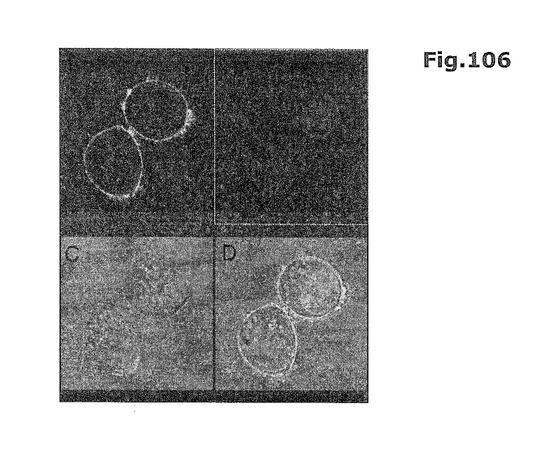

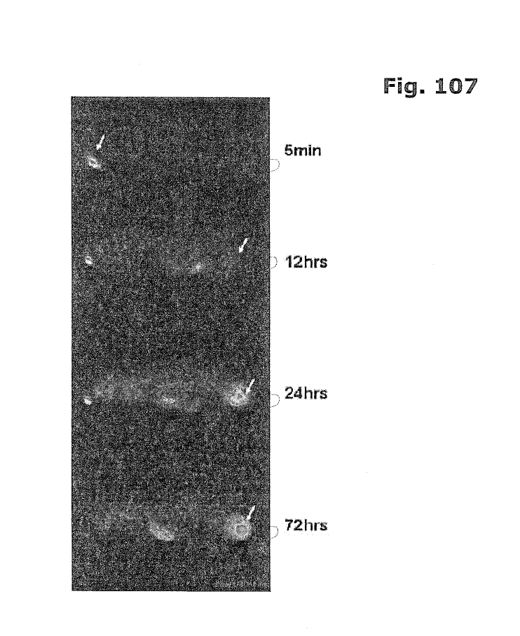

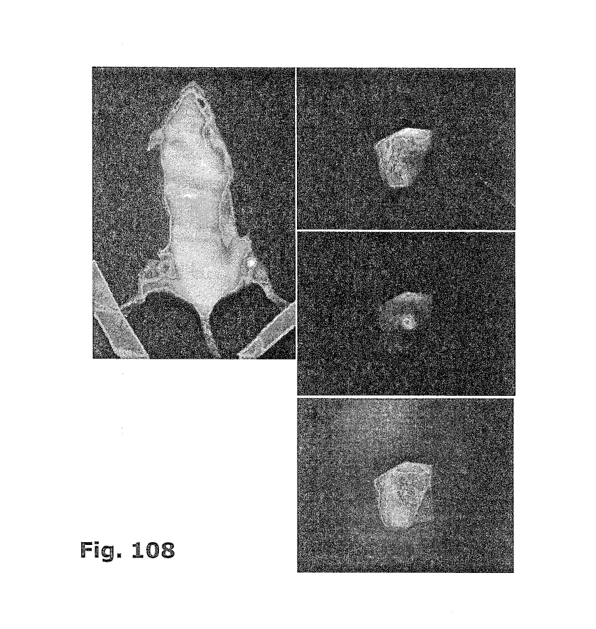

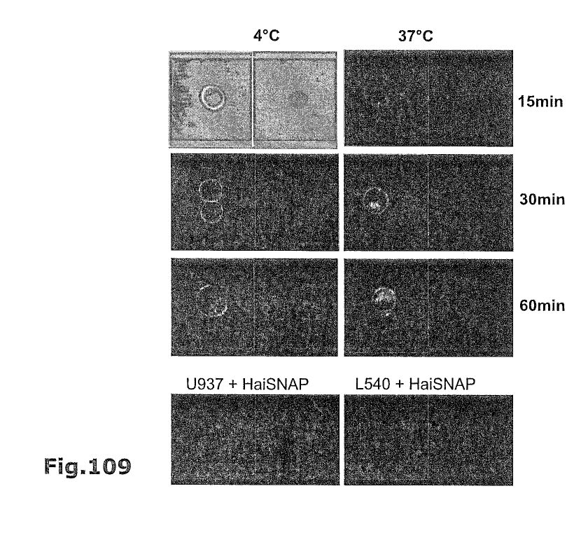

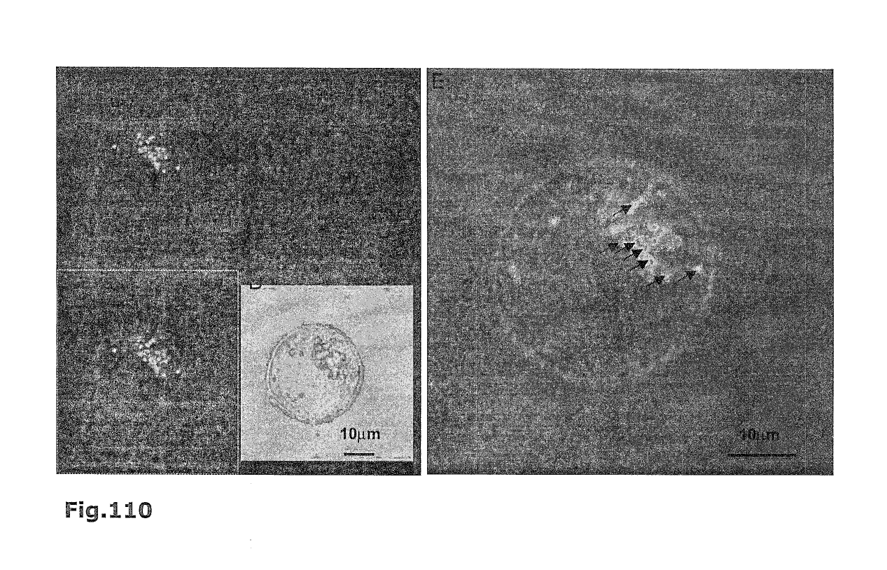

Part 1-3 represent a typical sandwich ELISA protocol as previously described. A schematic overview is given in FIG. 103).

The assay has to be performed in thin-walled polycarbonate plates suited for immunoassay as well as for thermocycling based applications (e.g. Nunc TopYield starter kit).

To avoid contamination by PCR product, handling of immuno-PCR product DNA is strictly separated from earlier immuno-PCR set-up steps, by performing both in different, well-separated laboratories.

The protocol is performed as follows:

The initial working step is mostly performed as described in the “Sandwich ELISA” protocol. The only difference is the use of thin walled polycarbonate PCR grade 96 well plates or strips instead of conventional ELISA plates.

Instead of fluorophor labeled detection antibody (SNAP-tag fusion protein) a DNA template coupled detection antibody complex is used.

To remove as much unbound target DNA the plates are rigorously washed five times with PBS+Tween (0.01%), soaking wells for 3 min with wash buffer during each cycle, followed by two washes with ultrapure, 0.2 mm filtered water. After addition of PCR reagents, the plates are subjected to 30 cycles of PCR amplification, using a 96-well real-time PCR cycler, for example the ABIprism 7000 (Applied Biosystems) system.

The TaqMan Universal PCR Mastermix is prepared according to the manufacturer's instructions using the described concentrations of primer-1, primer-2 and probe. 30 μl of the PCR Mastermix are pipetted in each well. The modules are sealed with an adhesive foil. The plate or PCR stripes are placed into the precleaned real time PCR machine and a typical PCR program is run: initial denaturation: 5 min 95° C. followed by 30 cycles consisting of denaturation step: 30 s, 50° C., synthesis step: 30 s, 72° C. and denaturation step: 12 s, 95° C. After the run the acquired data are evaluated by software.

### Flow Cytometric Applications of SNAP-Tag Fusion Proteins

The cell-binding activity of the SNAP-tag fusion proteins containing a targeting component A was evaluated using a FACS Calibur flow cytometer and CellQuest software (Becton Dickinson, Heidelberg, Germany) or the free software WinMDI 2.8. The SNAP-tag fusion protein was labeled ahead of application as described in “Labeling of SNAP-tag fusion proteins with BG derivatives of organic fluorophores”. Cells were labeled with the fluorophor labeled SNAP-tag complex by incubation for 30 minutes on ice. Cells were then washed twice with 500 μl cold PBS in an automated cell washer (Dade Serocent; Baxter). As alternative to the direct staining of the complex, the binding of the SNAP-tag fusion protein (only AB) was detected via the polyhistidine tag by using a Penta-His Alexa Fluor 488 antibody (Qiagen, Hilden, Germany).

In certain embodiments of this procedure antibody SNAP-tag fusion constructs targeting the human cell surface molecules CD30 (Hodgkin lymphoma), the CD64 ((Fc gamma RI) on activated macrophages) and the EGF receptor (on pancreatic-, breast-, lung- and non small cell lung cancer) were employed.

### a) Targeting CD30:

The scFv Ki4, a monomeric recombinant version of the parental CD30 specific monoclonal antibody (Barth e al., 2000) and its counterpart the CD30 ligand were cloned as fusion proteins to SNAP-tag. The choice of positions (N- or C-terminal) was done in respect of maintaining full functionality of both fusion partners. FIG. (105A) shows an evaluation of flow cytometric analysis of Ki-SNAP labeled with SNAP-vista Green (Covalys) binding on the CD30 overexpressing cell line L540cy.

Briefly 5×10⁵ L540cy cells were mixed with different amounts of SNAP-vista Green labeled Ki4-SNAP (5, 50 and 500 ng) in 500 μl PBS. The binding reaction was allowed to proceed for 20 minutes on ice in the dark. Afterwards the cells were washed twice with PBS and analyzed in a FACS Calibur (Becton&Dickinson) flow cytometer.

FIG. (105B) shows an evaluation of flow cytometric analysis of SNAP-CD30L labeled with SNAP-vista Green (Covalys) binding on the CD30 overexpressing cell line L540cy.

Briefly 5×10⁵ L540cy cells were mixed with 500 ng of Vista Green labeled SNAP-CD30L in 500 μl PBS. The binding reaction was allowed to proceed for 20 minutes on ice in the dark. Afterwards the cells were washed twice with PBS and analyzed in a FACS Calibur (beton&Dickinson) flow cytometer. As negative control the CD30 negative cell line L3.6 μl was stained and analyzed in the same manner.

### b) Targeting EGFR:

The scFv 425 (further named Hai), a monomeric recombinant version of the parental EGFR specific monoclonal antibody (Haisma et. al., 2000) and the natural EGFR ligand EGF were cloned as fusion proteins to SNAP-tag. The choice of positions (N- or C-terminal) was done in respect of maintaining full functionality of both fusion partners.

FIG. (105C) shows an evaluation of flow cytometric analysis of Hai-SNAP labeled with SNAP-vista Green (Covalys) binding on the EGFR overexpressing cell line A431.

Briefly 5×10⁵ A431 cells were mixed with 500 ng of SNAP-vista Green labeled HAi-SNAP in 500 μl PBS. The binding reaction was allowed to proceed for 20 minutes on ice in the dark. Afterwards the cells were washed twice with PBS and analyzed in a FACS Calibur (Becton&Dickinson) flow cytometer. As negative control the same staining was performed with the EGFR negative cell line Monomac.

FIG. (105D) shows an evaluation of flow cytometric analysis of SNAP-EGF labeled with SNAP-vista Green (Covalys) binding on the EGFR overexpressing cell line A431.

Briefly 5×10⁵ A431 cells were mixed with 500 ng of Vista Green labeled SNAP-EGF in 500 μl PBS. The binding reaction was allowed to proceed for 20 minutes on ice in the dark. Afterwards the cells were washed twice with PBS and analyzed in a FACS Calibur (Becton&Dickinson) flow cytometer. As negative control the same staining was performed with the EGFR negative cell line CHO K1.

### c) Targeting CD64:

The H22, a monomeric recombinant version of the parental anti CD64 specific monoclonal antibody (Tur et. al., 2003) was cloned as fusion proteins N-terminal to SNAP-tag.

Briefly 5×10⁵ U937 cells (CD64 positive) were mixed with 500 ng of SNAP-vista Green labeled H22-SNAP in 500 0 PBS. The binding reaction was allowed to proceed for 20 minutes on ice in the dark. Afterwards the cells were washed twice with PBS and analyzed in a FACS Calibur (beton&Dickinson) flow cytometer. As negative control the same staining was performed with the CD64 negative cell line L540.

FIG. (105E) shows an evaluation of flow cytometric analysis of H22-SNAP labeled with SNAP-vista Green (Covalys) binding on the CD64 overexpressing cell line U937 and not binding on the CD64 negative cell line L540.

### d) Targeting Pancreatic Cancer:

To destinguish between inflammatory pancreatitis and pancratic cancer a immunized murine scFv Phage Display library was depleted on pancreatitis derived cellular material followed by three rounds of Phage Display selection. one pancreatic cancer specific scFv clone (clone 14.1) was selected as specific binder for pancreatic cancer derived cell lines L3.6 μl and A431 and being negative on pancreatitis cell membranes.

In brief 5×10⁵ A431/L3.6 μl cells were mixed with SNAP-vista Green labeled 14.1-SNAP in 500 μl PBS. The binding reaction was allowed to proceed for 20 minutes on ice in the dark. Afterwards the cells were washed twice with PBS and analyzed in a FACS Calibur (Becton&Dickinson) flow cytometer. As negative control the same staining was performed with the EGFR negative cell line L540 see FIG. (105F).

### Confocal Microscopy Applications of SNAP-Tag Fusion Proteins

The target cells were prepared as described in “Flow cytometric applications of SNAP-tag fusion proteins” but were fixed with formaldehyde after the last washing step. Therefore 300 μl of an icecold 0.4% formaldehyde-PBS solution were added to the cells on ice and incubated for 30 minutes. The cells were washed once again with PBS in an automated cell washer. For counterstaining of nuclei, the cells were mixed with 2 μl of a 1/100 diluton of Draq5 (BioStatus, Leicestershire, UK). After 5 minutes incubation 10 μl of the cell suspension were mounted on a glass slide covered with glass coverslips and investigated with a Leica fluorescence (DMR) and Confocal microscope (TSC SP).

FIG. (106) shows confocal pictures of L540cy cells stained with BG505 (Covalys) labeled Ki4-SNAP.

### In Vivo Imaging

In vivo imaging of L3.6 μl (EGFR⁺) tumor xenograft was done with the Intas Cri Maestro In vivo imager.

L3.6 μl pancreatic carcinoma 5×10⁵ cells were injected intravenously in a female 6 week old SCID mouse and visualized after 1 week growth by retrobulbic injection of 70 μg Hai-SNAP labeled with BG-782 NIR dye. The labeling reaction was done beforehand as described in section “Labeling of SNP-tag fusion proteins with BG derivatives of organic fluorophores”. The imaging was done with anesthetized mice after 5 minutes, 12, 24 and 72 hours after injection of the Hai-SNAP-BG782 imaging agent.

FIG. (107) shows infrared pictures from the whole mouse that were taken and analyzed by spectral unmixing the signal from background with the Intas Cri-Maestro In vivo imager. A large tumor in the abdomen of the mouse could be well visualized by accumulation of Hai-SNAP. There is a clear movement of the injected tumor imaging substrate from the place of injection towards the tumor detectable within a time range of 24 hours.

FIG. (108) shows infrared pictures from the whole mouse that were taken and analyzed by spectral unmixing the signal from background with the Intas Cri-Maestro In vivo imager. In contrast to picture 10 stably EGFP expressing L3.6pl pancreatic carcinoma 5×10⁵ cells were injected under the skin at the right and left femoral region of a female 6 week old SCID mouse and visualized after 1 week growth by retrobulbic injection of 70 μg Hai-SNAP labled with BG-782 NIR dye. The labeling reaction was done beforehand as described in section “Labeling of SNP-tag fusion proteins with BG derivatives of organic fluorophores”. The imaging was done with anesthetized mouse after 24 past injection of the Hai-SNAP-BG782 imaging agent.

The green fluorescence and the infrared fluorescence signal clearly overlap when overlaying the corresponding pictures taken by the Intas Cri-Maestro In vivo imager.

### Receptor Internalization Studies Using SNAP-Tag Fusion Proteins

The EGFR-positive target cells were prepared as described in “Confocal microscopy applications of SNAP-tag fusion proteins” but were fixed with formaldehyde after different time points of SNAP-tag fusion protein application. Therefore 300 μl of an icecold 0.4% formaldehyde-PBS solution were added after 15, 30 and 60 minutes of incubation at 4° C./37° to the cells on ice and incubated for 30 minutes. The cells were washed once again with PBS in an automated cell washer. For counterstaining of nuclei, the cells were mixed with 2 μl of a 1/100 diluton of Draq5 (BioStatus, Leicestershire, UK). After 5 minutes incubation 10 μl of the cell suspension were mounted on a glass slide covered with glass coverslips and investigated with a Leica fluorescence (DMR) and Confocal microscope (TSC SP).

FIG. (109) shows confocal pictures of L3.6 μl cells stained with BG505 (Covalys) labeled Hai-SNAP. There is a clear higher internalization rate of bound Hai-SNAP into the cells when incubated at 37° in comparison to the 4° C. sample. EGFR negative cell lines like L540 and U937 were not stained under the same conditions.

FIG. (110) shows the colocalization of Hai-SNAP BG505 labeled and transferrin ALEXA 594 labeled after internalization (see black arrows in (FIG. 109E)). FIG. (109A) shows internalized HaiSNAP-BG505 in green; (FIG. 109B) clathrin-mediated internalization of transferrin-ALEXA594 in blue, (FIG. 109C) an overlay of A and B and D is an overlay of C with transmission light picture; (FIG. 109E): magnification of (FIG. 109D): arrows depict vesicles harboring both labeled transferrin and HaiSNAP-BG505. There is a high degree of overlapping localization of transferrin and HaiSNAP-BG505.

Transferrin is known to be internalized via clathrin supported internalization. This is also reported for EGFR internalization. The colocalization of Hai-SNAP and transferrin therefore indicates that the original internalization route of EGFR is not affected by bound Hai-SNAP.

### Use of SNAP-Tag Fusion Proteins for Flow Cytometry Based High Producing Strain Selection

Cell permeable SNAP-tag staining substrates like BG-430, BG-505, BG-DAF and TMR-Star (Covalys) can be used to specifically label SNAP-tag fusion proteins in living mammalian cells. In order to detect the expression rate of transiently transfected HEK 293 or CHO cells (with pMS based vectors, see FIGS. 1A+B and 5) the cells were incubated with cell permeable TMRstar.

The TMS-Star substrate was dissolved in DMSO according to the manufacturers (Covalys) instructions. 5 μM, TMR-Star was diluted to a final working concentration of 1 μM. Cells were labeled in the dark for 30 min at 37° C., then washed twice in medium and incubated for a further 30 min prior to imaging to allow diffusion of non-reacted substrate out of the cell. All steps were performed under a laminar flow and with sterile filtrated solutions.

For microscopic preevaluation 1×10⁵ TMR stained cells were counterstained with a 1:2000 dilution of DRAQ5™ solution for 2 min followed by a washing step with PBS. Thhe staining solution concentration and washing conditions affter TMR staining were carefully determined by microscopy until the TMR background in non or mock transfected cell lines was low enough to get a good signal to background ratio in the cells expressing SNAP-tag fusion protein.

The rest of the properly stained cells were sorted according to the strength of the TMR signal. Ten percent of the cells with strongest TMR signal were sorted and afterwards transfered into a new culture flask.

This procedure was repeated on demand to get a homogenous high producing cell population for scale up of the mammalian expression.

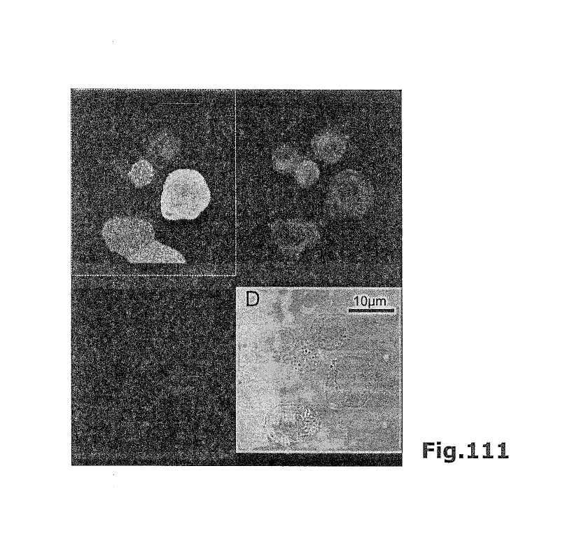

FIG. (111) shows the TMR staining of HEK293 cells expressing the Hai-SNAP fuison protein together with the EGFP reporter protein which is aencoded 3′ on the biscistronic mRNA. FIG. (111A) shows the signal of the EGFP reporter, (FIG. (111B) the TMR signal belonging to the SNAP-Tag fusion protein, (FIG. 111C) the Draq5 nuclear counterstain and (FIG. 111D) the transmission light picture of the same cells.

### Targeted Delivery of Interfering RNA Via SNAP-Tag Fusion Proteins

The coupling of anti eEFII siRNA and the Hai-SNAP fusion protein is basically done like described in section “Antibody-Nucleic acid conjugates (RNA)”.

The complex was applied to target cells in concentrations ranging from 10 ng/well (1×10⁵ target cells) to 100 ng/well (EGFR overexpressing cell line L3.6 μl) and the cells are tested for specific knockdown of target genes 62 hours after application of the siRNA complex by westen blot and quantitatve PCR.

This approach can readily be adapted to other ligands and ribonucleic acids based molecules e.g. miRNAs/shRNA of different specificity.

In a further application the coupling of RNA molecules is realized over a homotrifunctional or heterotrifunctional and homobifunctional/heterobifunctional crosslinkers containing a maleimide function, by which a RNA molecule (preferably having RNA interference properties like siRNA) containing a terminal primary SH-group is coupled.

The preformed crosslinker-RNA complex is then added in a molar ratio of 2:1 to the pre-purified SNAP-/CLIP-tag fusion proteins and reaced as described above. An example for heterobifunctional RNA bearing crosslinkers (one BG and on BC residue for crosslinking one SNAP-tag and one CLIP-tag fusion protein) is given in FIG. 77).

Examples for homotrifunctional RNA bearing crosslinkers (three BG residues for crosslinking of three SNAP-tag fusion proteins) is given in FIG. 10, 19, 50). This crosslinker (FIG. 50) additionally contains a fluorecein residue which enables tracing of the complex by e.g. microscopy.

### Targeted Delivery of Cytotoxic/Cytostatic Agents Via SNAP-Tag Fusion Proteins

In a specific embodiment of the invention the targeting complex AB is consisting of a EGFR targeting antibody (Hai) or natural lingand (EGF) whereas the SNAP-tag is coupled to Benzylguanine modified cytotoxic agents like Paclitaxel. This Paclitaxel is representing the invention related component C and is delivering in complex with AB a cytotoxic payload to targeted cells (EGFR overexpressing).

To increase the toxic payload per bound complex AB the toxic moiety is loaded on dendrimeric structures like described for Paclitaxel in Jongdoo Lim et al., 2007. or methothrexate in Gong Wu et al., 2006.

In brief the benzylguanine-modified dendrimers carrying a high number of cytotoxic moieties are coupled to the Hai-SNAP like described in section “Labeling of SNAP-tag fusion proteins with BG derivatives of organic fluorophores”. The purified (dialysis) dendrimer-Hai-SNAP complex is then applied to target cells and tested for specific cytotoxicity in a XTT based cell viability assay.

In a further imbodyment of the envention a toxic molecule like Chlorambucil is coupled to a homotrifunctional crosslinker. The preformed crosslinker-Chlorambucil complex is then added in a molar ratio of 3:1 to the pre-purified SNAP-/CLIP-tag fusion proteins and reaced as described above.

An example for heterotrifunctional Chlorambucil bearing crosslinker (three BG residues for crosslinking three SNAP-tag proteins) is given in FIGS. (17,43,51).

### Purification of Multitag Fusion Proteins Using SNAP-/CLIP-Tag and ACP-Tag Technology

The general structure of the protein complex is SNAP-/CLIP-tag-Protease cleavage site-Target protein (+His-Tag) −ACP-tag.

All steps are performed in a FPLC system to better monitor the protein concentrations of every protocol step. In brief, the protein is primarily covalently bound via SNAP-/CLIP-tag to SNAP-/CLIP-Capture purification resin. After this step, the resin is intensively washed until no protein signal can be detected in the wash fraction. By adding the desired protease the target protein is then cleaved off and released from the resin. The eluted protein is then directly bound to a IMAC (Ni-NTA) column to re-bind the target protein via His-Tag and remove the protease by simple washing steps. The Protein can then be labeled at the ACP-tag site with fluorophors etc. on the column. Afterwards unreacted ACP substrate is washed away and a labeled highly pure target protein can be eluted from the Ni-NTA column.

All of the methods and compositions disclosed and claimed herein can be made and executed without undue experimentations in light of the present disclosure. While the compositions and methods of this invention have been described in terms of preferred embodiments, it will be apparent to those of skill in the art that variations may be applied to the methods and the steps or in the sequence of the steps of the method described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

TABLE 1

| CD molecule | Alternate Names | Entrez Gene |
|---|---|---|
| CD1a | R4; HTA1 | 909 |
| CD1b | R1 | 910 |
| CD1c | M241; R7 | 911 |
| CD1d | R3 | 912 |
| CD1e | R2 | 913 |
| CD2 | CD2R; E-rosette receptor; T11; LFA-2 | 914 |
| CD3delta | CD3d | 915 |
| CD3epsilon | CD3e | 916 |
| CD3gamma | CD3g | 917 |
| CD4 | L3T4; W3/25 | 920 |
| CD5 | Leu-1; Ly-1; T1; Tp67 | 921 |
| CD6 | T12 | 923 |
| CD7 | gp40 | 924 |
| CD8alpha | Leu2; Lyt2; T cell co-receptor; T8 | 925 |
| CD8beta | Leu2; CD8; Lyt3 | 926 |
| CD9 | DRAP-27; MRP-1; p24 | 928 |
| CD10 | EC 3.4.24.11; neprilysin; CALLA; enkephalinase; gp100; | 4311 |
| | NEP | |
| CD11a | AlphaL integrin chain; LFA-1alpha | 3683 |
| CD11b | AlphaM integrin chain; AlphaM-beta2; C3biR; CR3; Mac-1; | 3684 |
| | Mo1 | |
| CD11c | AlphaX integrin chain; Axb2; CR4; leukocyte surface | 3687 |
| | antigen p150,95 | |
| CDw12 | p90-120 | 23444 |
| CD13 | APN; EC 3.4.11.2; gp150 | 290 |
| CD14 | LPS-R | 929 |
| CD15u | Sulphated CD15 | |
| CD16a | FCRIIIA | 2214 |
| CD16b | FCRIIIB | 2215 |
| CDw17 | LacCer | |
| CD18 | CD11a beta subunit; CD11b beta subunit; CD11c beta | 3689 |
| | subunit; beta-2 integrin chain | |
| CD19 | B4 | 930 |
| CD20 | B1; Bp35 | 931 |
| CD21 | C3d receptor; CR2; EBV-R | 1380 |
| CD22 | BL-CAM; Lyb8 | 933 |
| CD23 | B6; BLAST-2; FceRII; Leu-20; Low affinity IgE receptor | 2208 |
| CD24 | BA-1; HSA | 934 |
| CD25 | IL-2R alpha chain; IL-2R; Tac antigen | 3559 |
| CD26 | EC 3.4.14.5; ADA-binding protein; DPP IV ectoenzyme | 1803 |
| CD27 | S152; T14 | 939 |
| CD28 | T44; Tp44 | 940 |
| CD29 | Platelet GPIIa; VLA-beta chain; beta-1 integrin chain | 3688 |
| CD30 | Ber-H2 antigen; Ki-1 antigen | 943 |
| CD31 | GPiia′; endocam; PECAM-1 | 5175 |
| CD32 | FCR II; Fc gamma RII | 2212 |
| CD33 | gp67; p67 | 945 |
| CD34 | gp105-120 | 947 |
| CD35 | C3bR; C4bR; CR1; Immune Adherence Receptor | 1378 |
| CD36 | GPIIIb; GPIV; OKM5-antigen; PASIV | 948 |
| CD37 | gp52-40 | 951 |
| CD38 | T10; cyclic ADP-ribose hydrolase | 952 |
| CD39 | | 953 |
| CD40 | Bp50 | 958 |
| CD41 | GPIIb; alpha IIb integrin chain | 3674 |
| CD42a | GPIX | 2815 |
| CD42b | GPIbalpha; Glycocalicin | 2811 |
| CD42c | GPIb-beta | 2812 |
| CD42d | GPV | 2814 |
| CD43 | gpL115; leukocyte sialoglycoprotein; leukosialin; | 6693 |
| | sialophorin | |
| CD44 | ECMR III; H-CAM; HUTCH-1; Hermes; Lu, In-related; Pgp- | 960 |
| | 1; gp85 | |
| CD44R | CD44v; CD44v9 | 960 |
| CD45 | B220; CD45R; CD45RA; CD45RB; CD45RC; CD45RO; EC | 5788 |
| | 3.1.3.4; LCA; T200; Ly5 | |
| CD46 | MCP | 4179 |
| CD47R | Rh-associated protein; gp42; IAP; neurophilin; OA3; MEM- | 961 |
| | 133; formerly CDw149 | |
| CD48 | BCM1; Blast-1; Hu Lym3; OX-45 | 962 |
| CD49a | Alpha-1 integrin chain; VLA-1 alpha chain | 3672 |
| CD49b | Alpha-2 integrin chain; GPIa; VLA-2 alpha chain | 3673 |
| CD49c | Alpha-3 integrin chain; VLA-3 alpha chain | 3675 |
| CD49d | Alpha-4 integrin chain; VLA-4 alpha chain | 3676 |
| CD49e | Alpha-5 integrin chain; FNR alpha chain; VLA-5 alpha chain | 3678 |
| CD49f | Alpha-6 integrin chain; Platelet gpI; VLA-6 alpha chain | 3655 |
| CD50 | ICAM-3 | 3385 |
| CD51 | VNR-alpha chain; alpha V integrin chain; vitronectin | 3685 |
| | receptor | |
| CD52 | | 1043 |
| CD53 | | 963 |
| CD54 | ICAM-1 | 3383 |
| CD55 | DAF | 1604 |
| CD56 | Leu-19; NKH1; NCAM | 4684 |
| CD57 | HNK1; Leu-7 | 964 |
| CD58 | LFA-3 | 965 |
| CD59 | 1F-5Ag; H19; HRF20; MACIF; MIRL; P-18; Protectin | 966 |
| CD60a | GD3 | |
| CD60b | 9-O-acetyl-GD3 | |
| CD60c | 7-O-acetyl-GD3 | |
| CD61 | CD61A; GPIIb/IIIa; beta 3 integrin chain | 3690 |
| CD62E | E-selectin; ELAM-1; LECAM-2 | 6401 |
| CD62L | L-selectin; LAM-1; LECAM-1; Leu-8; MEL-14; TQ-1 | 6402 |
| CD62P | P-selectin; GMP-140; PADGEM | 6403 |
| CD63 | LIMP; MLA1; PTLGP40; gp55; granulophysin; LAMP-3; | 967 |
| | ME491; NGA | |
| CD64 | FC gammaRI; FCR I | 2209 |
| CD65 | Ceramide-dodecasaccharide; VIM-2 | |
| CD65s | Sialylated-CD65; VIM2 | |
| CD66a | NCA-160; BGP | 634 |
| CD66b | CD67; CGM6; NCA-95 | 1088 |
| CD66c | NCA; NCA-50/90 | 4680 |
| CD66d | CGM1 | 1084 |
| CD66e | CEA | 1048 |
| CD66f | Pregnancy specific b1 glycoprotein; SP-1; PSG | 5669 |
| CD68 | gp110; macrosialin | 968 |
| CD69 | AIM; EA 1; MLR3; gp34/28; VEA | 969 |
| CD70 | CD27-ligand; Ki-24 antigen | 970 |
| CD71 | T9; transferrin receptor | 7037 |
| CD72 | Ly-19.2; Ly-32.2; Lyb-2 | 971 |
| CD73 | Ecto-5′-nucleotidase | 4907 |
| CD74 | Class II-specific chaperone; Ii; Invariant chain | 972 |
| CD75 | Lactosamines | |
| CD75s | Alpha-2,6-sialylated lactosamines (formerly CDw75 and | |
| | CDw76) | |
| CD77 | Pk blood group antigen; BLA; CTH; Gb3 | |
| CD79a | Ig alpha; MB1 | 973 |
| CD79b | B29; Ig beta | 974 |
| CD80 | B7; BB1 | 941 |
| CD81 | TAPA-1 | 975 |
| CD82 | 4F9; C33; IA4; KAI1; R2 | 3732 |
| CD83 | HB15 | 9308 |
| CD84 | | 8832 |
| CD85 | ILT/LIR family | 10859 |
| CD86 | B7-2; B70 | 942 |
| CD87 | uPAR | 5329 |
| CD88 | C5aR | 728 |
| CD89 | Fcalpha-R; IgA Fc receptor; IgA receptor | 2204 |
| CD90 | Thy-1 | 7070 |
| CD91 | ALPHA2M-R; LRP | 4035 |
| CD92 | CTL1; formerly CDw92 | 23446 |
| CDw93 | | 23447 |
| CD94 | Kp43 | 3824 |
| CD95 | APO-1; Fas; TNFRSF6; APT1 | 355 |
| CD96 | TACTILE | 10225 |
| CD97 | | 976 |
| CD98 | 4F2; FRP-1; RL-388 | 4198 |
| CD99 | CD99R; E2; MIC2 gene product | 4267 |
| CD100 | SEMA4D | 10507 |
| CD101 | IGSF2; P126; V7 | 9398 |
| CD102 | ICAM-2 | 3384 |
| CD103 | ITGAE; HML-1; integrin alphaE chain | 3682 |
| CD104 | beta 4 integrin chain; TSP-1180; beta 4 | 3691 |
| CD105 | endoglin | 2022 |
| CD106 | INCAM-110; VCAM-1 | 7412 |
| CD107a | LAMP-1 | 3916 |
| CD107b | LAMP-2 | 3920 |
| CD108 | SEMA7A; JMH human blood group antigen; formerly | 8482 |
| | CDw108 | |
| CD109 | 8A3; E123; 7D1 | |
| CD110 | MPL; TPO-R; C-MPL | 4352 |
| CD111 | PVRL1; PRR1; HevC; nectin-1; HIgR | 5818 |
| CD112 | HVEB; PRR2; PVRL2; nectin 2 | 5819 |
| CDw113 | PVRL3, Nectin3; poliovirus receptor-related 3; nectin-3 | 25945 |
| CD114 | CSF3R; HG-CSFR; G-CSFR | 1441 |
| CD115 | c-fms; CSF-1R; M-CSFR | 1436 |
| CD116 | GM-CSF receptor alpha chain | 1438 |
| CD117 | c-KIT; SCFR | 3815 |
| CD118 | LIFR; leukemia inhibitory factor receptor | 3977 |
| CDw119 | IFNgR; IFNgRa | 3459 |
| CD120a | TNFRI; p55 | 7132 |
| CD120b | TNFRII; p75; TNFR p80 | 7133 |
| CD121a | IL-1R; type 1 IL-1R | 3554 |
| CDw121b | IL-1R, type 2 | 7850 |
| CD122 | IL-2Rbeta | 3560 |
| CD123 | IL-3Ralpha | 3563 |
| CD124 | IL-4R | 3566 |
| CDw125 | IL-5Ralpha | 3568 |
| CD126 | IL-6R | 3570 |
| CD127 | IL-7R; IL-7R alpha; p90 Il7 R | 3575 |
| CDw128a | CXCR1; IL-8RA | 3577 |
| CDw128b | CXCR2; IL-8RB | 3579 |
| CD129 | Reserved | |
| CD130 | gp130 | 3572 |
| CD131 | common beta subunit | 1439 |
| CD132 | IL2RG; common cytokine receptor gamma chain; common | 3561 |
| | gamma chain | |
| CD133 | PROML1; AC133; hematopoietic stem cell antigen; | 8842 |
| | prominin-like 1 | |
| CD134 | OX40 | 7293 |
| CD135 | flt3; Flk-2; STK-1 | 2322 |
| CDw136 | msp receptor; ron; p158-ron | 4486 |
| CDw137 | 4-1BB; ILA | 3604 |
| CD138 | heparan sulfate proteoglycan; syndecan-1 | 6382 |
| CD139 | | 23448 |
| CD140a | PDGF-R; PDGFRa | 5156 |
| CD140b | PDGFRb | 5159 |
| CD141 | fetomodulin; TM | 7056 |
| CD142 | F3; coagulation Factor III; thromboplastin; TF | 2152 |
| CD143 | EC 3.4.15.1; ACE; kininase II; peptidyl dipeptidase A | 1636 |
| CD144 | cadherin-5; VE-Cadherin | 1003 |
| CDw145 | | |
| CD146 | MCAM; A32; MUC18; Mel-CAM; S-endo | 4162 |
| CD147 | 5A11; Basigin; CE9; HT7; M6; Neurothelin; OX-47; | 682 |
| | EMMPRIN; gp42 | |
| CD148 | HPTP-eta; DEP-1; p260 | 5795 |
| CDw149 | new designation is CD47R | |
| CD150 | SLAM; IPO-3; fomerly CDw150 | 6504 |
| CD151 | PETA-3; SFA-1 | 977 |
| CD152 | CTLA-4 | 1493 |
| CD153 | CD30L | 944 |
| CD154 | CD40L; T-BAM; TRAP; gp39 | 959 |
| CD155 | PVR | 5817 |
| CD156a | ADAM8; MS2 human; fomerly CD156 | 101 |
| CD156b | ADAM17; TACE; cSVP | 6868 |
| CDw156C | ADAM10; a disintegrin and metalloproteinase domain 10 | 102 |
| CD157 | BP-3/IF-7; BST-1; Mo5 | 683 |
| CD158 | KIR family | |
| CD159a | NKG2A | 3821 |
| CD159c | NKG2C; killer cell lectin-like receptor subfamily C, member 2 | 3822 |
| CD160 | BY55 antigen; NK1; NK28 | 11126 |
| CD161 | KLRB1; NKR-P1A; killer cell lectin-like receptor subfamily | 3820 |
| | B, member 1 | |
| CD162 | PSGL-1, PSGL | 6404 |
| CD162R | PEN5 (a post-translational modification of PSGL-1) | 6404 |
| CD163 | GHI/61; M130; RM3/1 | 9332 |
| CD164 | MUC-24; MGC-24v | 8763 |
| CD165 | AD2; gp37 | 23449 |
| CD166 | BEN; DM-GRASP; KG-CAM; Neurolin; SC-1; ALCAM | 214 |
| CD167a | trkE; trk6; cak; eddr1; DDR1; MCK10; RTK6; NTRK4 | 780 |
| CD168 | HMMR; IHABP; RHAMM | 3161 |
| CD169 | sialoadhesin; siglec-1 | 6614 |
| CD170 | Siglec-5 | 8778 |
| CD171 | L1; L1CAM; N-CAM L1 | 3897 |
| CD172a | SIRP alpha | 8194 |
| CD172b | SIRPbeta; signal-regulatory protein beta 1 | 10326 |
| CD172g | SIRPgamma; signal-regulatory protein beta 2 | 55423 |
| CD173 | Blood group H type 2 | |
| CD174 | Lewis y | 2525 |
| CD175 | Tn | |
| CD175s | Sialyl-Tn | |
| CD176 | TF | |
| CD177 | NB1 | |
| CD178 | fas-L; TNFSF6; APT1LG1; CD95-L | 356 |
| CD179a | VpreB; VPREB1; IGVPB | 7441 |
| CD179b | IGLL1; lambda5; immunoglobulin omega polypeptide; | 3543 |
| | IGVPB; 14.1 chain | |
| CD180 | LY64; RP105 | 4064 |
| CD181 | CXCR1; (was CDw128A), IL8Ralpha | 3577 |
| CD182 | CXCR2; (was CDw128B), IL8Rbeta | 12765 |
| CD183 | CXCR3; GPR9; CKR-L2; IP10-R; Mig-R | 2833 |
| CD184 | CXCR4; fusin; LESTR; NPY3R; HM89; FB22 | 7852 |
| CD185 | CXCR5; Chemokine (C—X—C motif) Receptor 5, Burkitt | 643 |
| | lymphoma receptor 1 | |
| CDw186 | CXCR6; Chemokine (C—X—C motif) Receptor 6 | 10663 |
| CD191 | CCR1; Chemokine (C-C motif) Receptor 1, RANTES | 1230 |
| | Receptor | |
| CD192 | CCR2; Chemokine (C-C motif) Receptor 2, MCP-1 receptor | 1231 |
| CD193 | CCR3; Chemokine (C-C motif) Receptor 3, eosinophil | 1232 |
| | eotaxin receptor | |
| CD195 | CCR5 | 1234 |
| CD196 | CCR6; Chemokine (C-C motif) Receptor 6 | 1235 |
| CD197 | CCR7; (was CDw197) Chemokine (C-C motif) Receptor 7 | 1236 |
| CDw198 | CCR8; Chemokine (C-C motif) Receptor 8 | 1237 |
| CDw199 | CCR9; Chemokine (C-C motif) Receptor 9 | 10803 |
| CDw197 | CCR7 | 1236 |
| CD200 | OX2 | 4345 |
| CD201 | EPC R | 10544 |
| CD202b | tie2; tek | 7010 |
| CD203c | NPP3; PDNP3; PD-Ibeta; B10; gp130RB13-6; ENPP3; | 5169 |
| | bovine intestinal phosphodiesterase | |
| CD204 | macrophage scavenger R | 4481 |
| CD205 | DEC205 | 4065 |
| CD206 | MRC1; MMR | 4360 |
| CD207 | Langerin | 50489 |
| CD208 | DC-LAMP | 27074 |
| CD209 | DC-SIGN | 30385 |
| CDw210 | IL-10 R | 3587; 3588 |
| CD212 | IL-12 R | 3594 |
| CD213a1 | IL-13 R alpha 1 | 3597 |
| CD213a2 | IL-13 R alpha 2 | 3598 |
| CDw217 | IL-17 R | 23765 |
| CDw218a | IL18Ralpha; IL18Ralpha | |
| CDw218b | IL18Rbeta; IL18Rbeta | |
| CD220 | Insulin R | 3643 |
| CD221 | IGF1 R | 3480 |
| CD222 | Mannose-6-phosphate/IGF2 R | 3482 |
| CD223 | LAG-3 | 3902 |
| CD224 | GGT; EC2.3.2.2 | 2678 |
| CD225 | Leu13 | 8519 |
| CD226 | DNAM-1; PTA1; TLiSA1 | 10666 |
| CD227 | MUC1; episialin; PUM; PEM; EMA; DF3 antigen; H23 | 4582 |
| | antigen | |
| CD228 | melanotransferrin | 4241 |
| CD229 | Ly9 | 4063 |
| CD230 | Prion protein | 5621 |
| CD231 | TM4SF2; A15; TALLA-1; MXS1; CCG-B7; TALLA | 7102 |
| CD232 | VESP R | 10154 |
| CD233 | band 3; erythrocyte membrane protein band 3; AE1; | 6521 |
| | SLC4A1; Diego blood group; EPB3 | |
| CD234 | Fy-glycoprotein; Duffy antigen | 2532 |
| CD235a | Glycophorin A | 2993 |
| CD235b | Glycophorin B | 2994 |
| CD235ab | Glycophorin A/B crossreactive mabs | |
| CD236 | Glycophorin C/D | |
| CD236R | Glycophorin C | 2995 |
| CD238 | Kell | 3792 |
| CD239 | B-CAM | 4059 |
| CD240CE | Rh30CE | 6006 |
| CD240D | Rh30D | 6007 |
| CD240DCE | Rh30D/CE crossreactive mabs | |
| CD241 | RhAg | 6005 |
| CD242 | ICAM-4 | 3386 |
| CD243 | MDR-1 | 5243 |
| CD244 | 2B4; NAIL; p38 | 51744 |
| CD245 | p220/240 | |
| CD246 | Anaplastic lymphoma kinase | 238 |
| CD247 | Zeta chain | 919 |
| CD248 | TEM1, Endosialin; CD164 sialomucin-like 1, tumor | 57124 |
| | endothelial marker 1 | |
| CD249 | Aminopeptidase A; APA, gp160 | 2028 |
| CD252 | OX40L; TNF (ligand) superfamily member 4, CD134 ligand | 7292 |
| CD253 | TRAIL; TNF (ligand) superfamily member 10, APO2L | 8743 |
| CD254 | TRANCE; TNF (ligand) superfamily member 11, RANKL | 8600 |
| CD256 | APRIL; TNF (ligand) superfamily member 13, TALL2 | 8741 |
| CD257 | BLYS; TNF (ligand) superfamily, member 13b, TALL1, BAFF | 10673 |
| CD258 | LIGHT; TNF (ligand) superfamily, member 14 | 8740 |
| CD261 | TRAIL-R1; TNFR superfamily, member 10a, DR4, APO2 | 8797 |
| CD262 | TRAIL-R2; TNFR superfamily, member 10b, DR5 | 8795 |
| CD263 | TRAIL-R3; TNFR superfamily, member 10c, DCR1 | 8794 |
| CD264 | TRAIL-R4; TNFR superfamily, member 10d, DCR2 | 8793 |
| CD265 | TRANCE-R; TNFR superfamily, member 11a, RANK | 8792 |
| CD266 | TWEAK-R; TNFR superfamily, member 12A, type I | 51330 |
| | transmembrane protein Fn14 | |
| CD267 | TACI; TNFR superfamily, member 13B, transmembrane | 23495 |
| | activator and CAML interactor | |
| CD268 | BAFFR; TNFR superfamily, member 13C, B cell-activating | 115650 |
| | factor receptor | |
| CD269 | BCMA; TNFR superfamily, member 17, B-cell maturation | 608 |
| | factor | |
| CD271 | NGFR (p75); nerve growth factor receptor (TNFR | 4804 |
| | superfamily, member 16) | |
| CD272 | BTLA; B and T lymphocyte attenuator | 151888 |
| CD273 | B7DC, PDL2; programmed cell death 1 ligand 2 | 80380 |
| CD274 | B7H1, PDL1; programmed cell death 1 ligand 1 | 29126 |
| CD275 | B7H2, ICOSL; inducible T-cell co-stimulator ligand (ICOSL) | 23308 |
| CD276 | B7H3; B7 homolog 3 | 80381 |
| CD277 | BT3.1; B7 family: butyrophilin, subfamily 3, member A1 | 11119 |
| CD278 | ICOS; inducible T-cell co-stimulator | 29851 |
| CD279 | PD1; programmed cell death 1 | 5133 |
| CD280 | ENDO180; uPARAP, mannose receptor, C type 2, TEM22 | 9902 |
| CD281 | TLR1; TOLL-like receptor 1 | 7096 |
| CD282 | TLR2; TOLL-like receptor 2 | 7097 |
| CD283 | TLR3; TOLL-like receptor 3 | 7098 |
| CD284 | TLR4; TOLL-like receptor 4 | 7099 |
| CD289 | TLR9; TOLL-like receptor 9 | 54106 |
| CD292 | BMPR1A; Bone Morphogenetic Protein Receptor, type IA | 657 |
| CDw293 | BMPR1B; Bone Morphogenetic Protein Receptor, type IB | 658 |
| CD294 | CRTH2; PGRD2; G protein-coupled receptor 44, | 11251 |
| CD295 | LEPR; Leptin Receptor | 3953 |
| CD296 | ART1; ADP-ribosyltransferase 1 | 417 |
| CD297 | ART4; ADP-ribosyltransferase 4; Dombrock blood group | 420 |
| | glycoprotein | |
| CD298 | ATP1B3; Na+/K+-ATPase beta 3 subunit | 483 |
| CD299 | DCSIGN-related; CD209 antigen-like, DC-SIGN2, L-SIGN | 10332 |
| CD300a | CMRF35 FAMILY; CMRF-35H | 11314 |
| CD300c | CMRF35 FAMILY; CMRF-35A | 10871 |
| CD300e | CMRF35 FAMILY; CMRF-35L1 | |
| CD301 | MGL; CLECSF14, macrophage galactose-type C-type lectin | 10462 |
| CD302 | DCL1; Type I transmembrane C-type lectin receptor DCL-1 | 9936 |
| CD303 | BDCA2; C-type lectin, superfamily member 11 | 170482 |
| CD304 | BDCA4; Neuropilin 1 | 8829 |
| CD305 | LAIR1; Leukocyte-Associated Ig-like Receptor 1 | 3903 |
| CD306 | LAIR2; Leukocyte-Associated Ig-like Receptor 2 | 3904 |
| CD307 | IRTA2; Immunoglobulin superfamily Receptor | 83416 |
| | Translocation Associated 2 | |
| CD309 | VEGFR2; KDR (a type III receptor tyrosine kinase) | 3791 |
| CD312 | EMR2; EGF-like module containing, mucin-like, hormone | 30817 |
| | receptor-like 2 | |
| CD314 | NKG2D; Killer cell lectin-like receptor subfamily K, member 1 | 22914 |
| CD315 | CD9P1; Prostaglandin F2 receptor negative regulator | 5738 |
| CD316 | EWI2; Immunoglobulin superfamily, member 8 | 93185 |
| CD317 | BST2; Bone Marrow Stromal cell antigen 2 | 684 |
| CD318 | CDCP1; CUB domain-containing protein 1 | 64866 |
| CD319 | CRACC; SLAM family member 7 | 57823 |
| CD320 | 8D6; 8D6 Antigen; FDC | 51293 |
| CD321 | JAM1; F11 receptor | 50848 |
| CD322 | JAM2; Junctional Adhesion Molecule 2 | 58494 |
| CD324 | E-Cadherin; cadherin 1, type 1, E-cadherin (epithelial) | 999 |
| CDw325 | N-Cadherin; cadherin 2, type 1, N-cadherin (neuronal) | 1000 |
| CD326 | Ep-CAM; tumor-associated calcium signal transducer 1 | 4072 |
| CDw327 | siglec6; sialic acid binding Ig-like lectin 6 | 946 |
| CDw328 | siglec7; sialic acid binding Ig-like lectin 7 | 27036 |
| CDw329 | siglec9; sialic acid binding Ig-like lectin 9 | 27180 |
| CD331 | FGFR1; Fibroblast Growth Factor Receptor 1 | 2260 |
| CD332 | FGFR2; Fibroblast Growth Factor Receptor 2 (keratinocyte | 2263 |
| | growth factor receptor) | |
| CD333 | FGFR3; Fibroblast Growth Factor Receptor 3 | 2261 |
| | (achondroplasia, thanatophoric dwarfism) | |
| CD334 | FGFR4; Fibroblast Growth Factor Receptor 4 | 2264 |
| CD335 | NKp46; NCR1, (Ly94); natural cytotoxicity triggering | 9437 |
| | receptor 1 | |
| CD336 | NKp44; NCR2, (Ly95); natural cytotoxicity triggering | 9436 |
| | receptor 2 | |
| CD337 | NKp30; NCR3 | 259197 |
| CDw338 | ABCG2; ATP-binding cassette, sub-family G (WHITE), | 9429 |
| | member 2 | |
| CD339 | Jagged-1; Jagged 1 (Alagille syndrome) | 182 |

TABLE 2
Extracted from R. Thorpe et al., Cytokine 21 (2003) 48-49

| | | | | |
|---|---|---|---|---|
| Systematic name | Human chromosome | Human ligand | Mouse ligand | Chemokine receptors(s) |

CXC chemokine/receptor family

| | | | | |
|---|---|---|---|---|
| CXCL1 | 4q21.1 | GROα/MGSA-α | GRO/MIP-2/KC? | CXCR2 > CXCR1 |
| CXCL2 | 4q21.1 | GROβ/MGSA-β | GRO/MIP-2/KC? | CXCR2 |
| CXCL3 | 4q21.1 | GROγ/MGSA-γ | GRO/MIP-2/KC? | CXCR2 |
| CXCL4 | 4q21.1 | PF4 | PF4 | Unknown |
| CXCL5 | 4q21.1 | ENA-78 | GCP-2/LIX? | CXCR2 |
| CXCL6 | 4q21.1 | GCP-2 | GCP-2/LIX? | CXCR1, CXCR2 |
| CXCL7 | 4q21.1 | NAP-2 | Unknown | CXCR2 |
| CXCL8 | 4q21.1 | IL-8 | Unknown | CXCR1, CXCR2 |
| CXCL9 | 4q21.1 | Mig | Mig | CXCR3^{a} |
| CXCL10 | 4q21.1 | IP-10 | IP-10/CRG-2 | CXCR3^{a} |
| CXCL11 | 4q21.1 | I-TAC | I-TAC | CXCR3^{a} |
| CXCL12 | 10q11.21 | SDF-1 α/β | SDF-1/PBSF | CXCR4^{b} |
| CXCL13 | 4q21.1 | BCA-1 | BLC | CXCR5 |
| CXCL14 | 5q31.1 | BRAK/bolekine | BRAK | Unknown |
| (CXCL15) | | Unknown | Lungkine/WECHE | Unknown |
| CXCL16 | 17p13 | | | CXCR6 |

C chemokine/receptor family

| | | | | |
|---|---|---|---|---|
| XCL1 | 1q24.2 | Lymphotactin/SCM-1α/ | Lymphotactin | XCR1 |
| | | ATAC | | |
| XCL2 | 1q24.2 | SCM-1β | Unknown | XCR1 |

CX₃C chemokine/receptor family

| | | | | |
|---|---|---|---|---|
| CX3CL1 | 16q13 | Fractalkine | Neurotactin/ABCD-3 | CX3CR1 |

CC chemokine/receptor family

| | | | | |
|---|---|---|---|---|
| CCL1 | 17q11.2 | 1-309 | TCA-3/P500 | CCR8 |
| CCL2 | 17q11.2 | MCP-1/MCAF/TDCF | JE? | CCR2 |
| CCL3 | 17q12 | MIP-1α/LD78α | MIP-1α | CCR1, CCR5 |
| CCL3L1 | 17q12 | LD78β | Unknown | CCR1, CCR5 |
| CCL4 | 17q12 | MIP-1β | MIP-1β | CCR5³ |
| CCL5 | 17q12 | RANTES | RANTES | CCR1, CCR3, CCR5^{c} |
| (CCL6) | | Unknown | C10/MRP-1 | Unknown |
| CCL7 | 17q11.2 | MCP-3 | MARC? | CCR1, CCR2, CCR3 |
| CCL8 | 17q11.2 | MCP-2 | MCP-2? | CCR3, CCR5^{c} |
| (CCL9/10) | | Unknown | MRP-2/CCF18/MIP-1γ | CCR1 |
| CCL11 | 17q11.2 | Eotaxin | Eotaxin | CCR3 |
| (CCL12) | | Unknown | MCP-5 | CCR2 |
| CCL13 | 17q11.2 | MCP-4 | Unknown | CCR2, CCR3 |
| CCL14 | 17q12 | HCC-1 | Unknown | CCR1, CCR5 |
| CCL15 | 17q12 | HCC-2/Lkn-1/MIP-1 | Unknown | CCR1, CCR3 |
| CCL16 | 17q12 | HCC-4/LEC/LCC-1 | Unknown | CCR1, CCR2 |
| CCL17 | 16q13 | TARC | TARC/ABCD-2 | CCR4 |
| CCL18 | 17q12 | DC-CK1/PARC/AMAC-1 | Unknown | Unknown |
| CCL19 | 9p13.3 | MIP-3β/ELC/exodus-3 | MIP-3β/ELC/exodus-3 | CCR7^{d} |
| CCL20 | 2q36.3 | MIP/3α/LARC/exodus-1 | MIP-3α/LARC/exodus-1 | CCR6 |
| CCL21 | 9p13.3 | 6Ckine/SLC/exodus-2 | 6Ckine/SLC/exodus-2/ | CCR7^{d} |
| | | | TCA-4 | |
| CCL22 | 16q13 | MDC/STCP-1 | ABCD-1 | CCR4 |
| CCL23 | 17q12 | MPIF-1/CKβ8/CKβ8-1 | Unknown | CCR1 |
| CCL24 | 7q11.23 | Eotaxin-2/MPIF-2 | MPIF-2 | CCR3 |
| CCL25 | 19p13.3 | TECK | TECK | CCR9 |
| CCL26 | 7q11.23 | Eotaxin-3 | Unknown | CCR3 |
| CCL27 | 9p13.3 | CTACK/ILC | ALP/CTACK/ILC/ESkine | CCR10 |
| CCL28 | 5p12 | MEC | | CCR3/CCR10 |

^{a}CD183.
^{b}CD184.
^{c}CD195.
^{d}CD_{w} 197.

TABLE 3

| Name | Source | Target receptors | Target cells | Function |
|---|---|---|---|---|
| IL-1 | macrophages, B | CD121a/IL1R1, | T helper cells | co-stimulation |
| | cells, | CD121b/IL1R2 | | |
| | monocytes, | | | |
| | dendritic cells | | | |
| | | | B cells | Maturation & |
| | | | | proliferation |
| | | | Nk cells | activation |
| | | | macrophages, | inflammation, |
| | | | endothelium, | small amounts |
| | | | other | induce acute phase |
| | | | | reaction, large |
| | | | | amounts induce |
| | | | | fever |
| IL-2 | TH1-cells | CD25/IL2RA, | activated T cells | stimulates growth |
| | | CD122/IL2RB, | and B cells, NK | and differentiation |
| | | CD132/IL2RG | cells, | of T cell response. |
| | | | macrophages, | Can be used in |
| | | | oligodendrocytes | immunotherapy to |
| | | | | treat cancer or |
| | | | | suppressed for |
| | | | | transplant |
| | | | | patients. |
| IL-3 | activated T | CD123/IL3RA, | hematopoietic | growth and |
| | helper cells[3], | CD131/IL3RB | stem cells | differentiation to |
| | mast cells, NK | | | e.g. erythrocytes, |
| | cells, | | | granulocytes |
| | endothelium, | | | |
| | eosinophils | | | |
| | | | mast cells | growth and |
| | | | | histamine release |
| IL-4 | TH2-cells, just | CD124/IL4R, | activated B cells | proliferation and |
| | activated naive | CD132/IL2RG | | differentiation, |
| | CD4+ cell, | | | IgG1 and IgE |
| | memory CD4+ | | | synthesis. |
| | cells, mast cells, | | | Important role in |
| | macrophages | | | allergic response |
| | | | | (IgE) |
| | | | T cells | proliferation |
| IL-5 | TH2-cells, mast | CD125/IL5RA, | eosinophils | production |
| | cells, eosinophils | CD131/IL3RB | | |
| | | | B cells | differentiation, IgA |
| | | | | production |
| IL-6 | macrophages, | CD126/IL6RA, | activated B cells | differentiation into |
| | TH2-cells, B | CD130/IR6RB | | plasma cells |
| | cells, astrocytes, | | | |
| | endothelium | | | |
| | | | plasma cells | antibody secretion |
| | | | hematopoietic | differentiation |
| | | | stem cells | |
| | | | T cells, others | induces acute |
| | | | | phase reaction, |
| | | | | hematopoiesis, |
| | | | | differentiation, |
| | | | | inflammation |
| IL-7 | bone marrow | CD127/IL7RA, | pre/pro-B cell, | involved in B, T, |
| | stromal cells and | CD132/IL2RG | pre/pro-T cell, | and NK cell |
| | thymus stromal | | NK cells | survival, |
| | cells | | | development, and |
| | | | | homeostasis, |
| | | | | ↑proinflammatory |
| | | | | cytokines |
| IL-8 | macrophages, | CXCR1/IL8RA, | neutrophils, | Neutrophil |
| | lymphocytes, | CXCR2/IL8RB/CD128 | basophils, | chemotaxis |
| | epithelial cells, | | lymphocytes | |
| | endothelial cells | | | |
| IL-9 | Th2-cells, | CD129/IL9R | T cells, B cells | Potentiates IgM, |
| | specifically by | | | IgG, IgE, |
| | CD4+ helper | | | stimulates mast |
| | cells | | | cells |
| IL-10 | monocytes, TH2- | CD210/IL10RA, | macrophages | cytokine |
| | cells, CD8+ T | CDW210B/IL10RB | | production |
| | cells, mast cells, | | | |
| | macrophages, B | | | |
| | cell subset | | | |
| | | | B cells | activation |
| | | | Th1 cells | inhibits Th1 |
| | | | | cytokine |
| | | | | production (IFN-γ, |
| | | | | TNF-β, IL-2) |
| | | | Th2 cells | Stimulation |
| IL-11 | bone marrow | IL11RA | bone marrow | acute phase |
| | stroma | | stroma | protein production, |
| | | | | osteoclast |
| | | | | formation |
| IL-12 | dendritic cells, B | CD212/IL12RB1, | activated [3] T | differentiation into |
| | cells, T cells, | IR12RB2 | cells, | Cytotoxic T cells |
| | macrophages | | | with IL-2[3], ↑ |
| | | | | IFN-γ, TNF-α, ↓ IL- |
| | | | | 10 |
| | | | NK cells | ↑ IFN-γ, TNF-α |
| IL-13 | activated TH2- | IL13R | TH2-cells, B | Stimulates growth |
| | cells, mast cells, | | cells, | and differentiation |
| | NK cells | | macrophages | of B-Cells (IgE), |
| | | | | inhibits TH1-cells |
| | | | | and the production |
| | | | | of macrophage |
| | | | | inflammatory |
| | | | | cytokines (e.g. IL- |
| | | | | 1, IL-6), ↓ IL-8, |
| | | | | IL-10, IL-12 |
| IL-14 | T cells and | | activated B cells | controls the |
| | certain | | | growth and |
| | malignant B cells | | | proliferation of B |
| | | | | cells, inhibits Ig |
| | | | | secretion |
| IL-15 | mononuclear | IL15RA | T cells, activated | Induces production |
| | phagocytes (and | | B cells | of Natural Killer |
| | some other | | | Cells |
| | cells), especially | | | |
| | macrophages | | | |
| | following | | | |
| | infection by | | | |
| | virus(es) | | | |
| IL-16 | lymphocytes, | CD4 | CD4+ T cells | CD4+ |
| | epithelial cells, | | | chemoattractant |
| | eosinophils, | | | |
| | CD8+ T cells | | | |
| IL-17 | subsets of T cells | CDw217/IL17RA, | epithelium, | osteoclastogenesis, |
| | | IL17RB | endothelium, | angiogenesis, ↑ |
| | | | other | inflammatory |
| | | | | cytokines |
| IL-18 | macrophages | CDw218a/IL18R1 | Th1 cells, NK | Induces production |
| | | | cells | of IFNγ, ↑ NK cell |
| | | | | activity |
| IL-19 | — | IL20R | | — |
| IL-20 | — | IL20R | | regulates |
| | | | | proliferation and |
| | | | | differentiation of |
| | | | | keratinocytes |
| IL-21 | — | IL21R | | |
| IL-22 | — | IL22R | | Activates STAT1 |
| | | | | and STAT3 and |
| | | | | increases |
| | | | | production of acute |
| | | | | phase proteins |
| | | | | such as serum |
| | | | | amyloid A, Alpha |
| | | | | 1- |
| | | | | antichymotrypsin |
| | | | | and haptoglobin in |
| | | | | hepatoma cell lines |
| IL-23 | — | IL23R | | Increases |
| | | | | angiogenesis but |
| | | | | reduces CD8 T-cell |
| | | | | infiltration |
| IL-24 | — | IL20R | | Plays important |
| | | | | roles in tumor |
| | | | | suppression, |
| | | | | wound healing and |
| | | | | psoriasis by |
| | | | | influencing cell |
| | | | | survival. |
| IL-25 | — | LY6E | | Induces the |
| | | | | production IL-4, |
| | | | | IL-5 and IL-13, |
| | | | | which stimulate |
| | | | | eosinophil |
| | | | | expansion |
| IL-26 | — | IL20R1 | | Enhances secretion |
| | | | | of IL-10 and IL-8 |
| | | | | and cell surface |
| | | | | expression of |
| | | | | CD54 on epithelial |
| | | | | cells |
| IL-27 | — | IL27RA | | Regulates the |
| | | | | activity of B |
| | | | | lymphocyte and T |
| | | | | lymphocytes |
| IL-28 | — | IL28R | | Plays a role in |
| | | | | immune defense |
| | | | | against viruses |
| IL-29 | — | | | Plays a role in host |
| | | | | defenses against |
| | | | | microbes |
| IL-30 | — | | | Forms one chain of |
| | | | | IL-27 |
| IL-31 | — | IL31RA | | May play a role in |
| | | | | inflammation of |
| | | | | the skin |
| IL-32 | — | | | Induces monocytes |
| | | | | and macrophages |
| | | | | to secrete TNF-α, |
| | | | | IL-8 and CXCL2 |
| IL-33 | — | | | Induces helper T |
| | | | | cells to produce |
| | | | | type 2 cytokine |
| IL-35 | regulatory T | | | Suppression of T |
| | cells | | | helper cell |
| | | | | activation |

### LITERATURE

1. Baneyx F, Georgiou G: Degradation of secreted proteins in Escherichia coli. Ann N Y Acad Sci 1992, 665:301-308.
2. Baskin J M, Prescher J A, Laughlin S T, Agard N J, Chang P V, Miller I A, Lo A, Codelli J A, Bertozzi C R: Copper-free click chemistry for dynamic in vivo imaging. Proc Natl Acad Sci USA 2007, 104:16793-16797.
3. Dyba M, Tarasova N I, Michejda C J: Small molecule toxins targeting tumor receptors. Curr Pharm Des 2004, 10:2311-2334.
4. GAUTIER A, JOHNSSON, K., KINDERMANN, M., JUILLERAT, A., BEAUFILS, F. PCT/EP2007/057597: LABELLING OF FUSION PROTEINS WITH SYNTHETIC PROBES. 2008.
5. Hochuli E: Large-scale chromatography of recombinant proteins. J Chromatogr 1988, 444:293-302.
6. JACCARD H, JOHNSSON, K., KINDERMANN, M., SIELAFF, I. C. PCT/EP2005/050900: SPECIFIC SUBSTRATES FOR O6-ALKYLGUANINE-DNA ALKYLTRANSFERASE. 2005.
7. JOHNSSON K, GEORGE, N. PCT/IB2004/001733: METHODS FOR PROTEIN LABELING BASED ON ACYL CARRIER PROTEIN. 2004.
8. KINDERMANN M, SCHWAB, M. PCT/EP2006/061798: PYRIMIDINES REACTING WITH O6-ALKYLGUANINE-DNA ALKYLTRANSFERASE. 2006.
9. Porath J, Carlsson J, Olsson I, Belfrage G: Metal chelate affinity chromatography, a new approach to protein fractionation. Nature 1975, 258:598-599.
10. Steinborn G, Boer E, Scholz A, Tag K, Kunze G, Gellissen G: Application of a wide-range yeast vector (CoMed) system to recombinant protein production in dimorphic Arxula adeninivorans, methylotrophic Hansenula polymorpha and other yeasts. Microb Cell Fact 2006, 5:33.
11. Stocker M, Tur M K, Sasse S, Krussmann A, Barth S, Engert A: Secretion of functional anti-CD30-angiogenin immunotoxins into the supernatant of transfected 293T-cells. Protein Expr Purif 2003, 28:211-219.
12. Thorpe, R., et al., Cytokine 21 (2003) 48-49

What is claimed is:

1. A method of diagnosis of a human or animal disorder, the method comprising
providing a compound to a human or an animal in a physiologically acceptable form, the compound comprising three components A, B, and C, which components are covalently bound forming the compound having the structure A-B-C, with component C providing a detectable label, with the compound being a heterologous complex comprising
at least one recombinant fusion protein comprising at least one each of the component A and the component B, with the component A comprising a cell-specific binding component and the component B comprising an enzymatic protein,
with at least one of the component C being covalently coupled to the component B,
wherein component A has a specific binding affinity for antigens and comprises antigen binding polypeptides targeting cell type specific markers,
component B is covalently linked to component A,
component C is a compound that comprises
before reaction with the enzymatic protein, at least one member of the group selected from an alkylated purine, a pyrimidine moiety, and coenzyme A (CoA) and
a moiety mediating biological activity in a targeted cell or organism thereby having a physiological effect, and
wherein component B has a catalytic or acceptor activity to couple the alkylated purine, the pyrimidine moiety, or the CoA of component C with covalently coupled components A-B.

2. The method of claim 1 wherein the compound is a heterologous complex comprising
at least one recombinant fusion protein comprising at least one each of the component A and the component B, with the component A providing binding to a soluble antigen and the component B comprising the enzymatic protein,
with at least one of the component C being covalently coupled to the component B.

3. The method of claim 1 wherein component A comprises a polypeptidic chemical moiety having an antigen binding structure, and component B comprises an enzymatic protein linked to component A.

4. The method of claim 1 wherein component A comprises moieties selected from the group consisting of antibodies, receptor ligands, enzyme substrates, lectins, cytokines, lymphokines, interleukins, angiogenic factors, virulence factors, allergens, peptidic allergens, recombinant allergens, allergen-idiotypical antibodies, autoimmune-provoking structures, tissue-rejection-inducing structures, immunoglobulin constant regions and derivatives, mutants or combinations thereof.

5. The method of claim 1 wherein component B is a polypeptide that reacts covalently with a specific substrate.

6. The method of claim 1 wherein component B is a derivative of human DNA repair protein O⁶-alkylguanine-DNA alkyltransferase (AGT).

7. The method of claim 1 wherein component B is a derivative of the Acyl Carrier Protein (ACP).

8. The method of claim 1 wherein the substrate for component B is O6-benzylguanine or O2-benzylcytosine.

9. The method of claim 1 wherein the covalently coupled components A-B are polypeptides.

10. The method of claim 1 wherein component C comprises a moiety which serves as a substrate for component B.

11. The method of claim 1 wherein component C comprises structure (X)ₙ₁-(Y)-(Z)ₙ₂ with
X being a component B specific substrate that comprises the alkylated purine and/or a pyrimidine moiety and n1 being one or more,
Z comprising the component mediating biological activity in a targeted cell or organism and n2 being 1 or more and
Y is a linker structural element to functionally connect X and Z.

12. The method of claim 11 wherein Y is a spacer between X and Z.

13. The method of claim 11 wherein the linker structural element comprises structural elements enabeling a controlled release of Z in response to a change in pH or upon exposure to as pH endosome or cytosol.

14. The method of claim 11 wherein the structural element Y comprises a polymeric structure selected from the group consisting of linear, branched, and tree like.

15. The method of claim 1 wherein the detectable label comprises a fluorescent molecule, a radioisotope, a quantum dot, a metal nanocluster, an iron oxide particle, or an enzyme used for detection.

16. A method of manufacturing the compound of claim 1 comprising reacting AB or BA with the component C, with the component C comprising
one or more enzyme substrates for which B is specific and one or more copies of a detectable label.


SEQUENCE LISTING (70 sequences)

SEQ ID NO: 1 - DNA, 1484 bp, Homo sapiens
atggagacagacacactcctgctatgggtactgctgctctgggttccaggttccactggtgacgcggcccagccggccatggcccaggtgcagctggtggagagcggtggaggtgttgtgcaacctggccggtccctgcgcctgtcctgctcctcgtctggcttcattttcagtgacaattacatgtattgggtgagacaggcacctggaaaaggtcttgagtgggttgcaaccattagtgatggtggtagttacacctactatccagacagtgtgaagggaagatttacaatatcgagagacaacagcaagaacacattgttcctgcaaatggacagcctgagacccgaagacaccggggtctatttttgtgcaagaggctactataggtacgagggggctatggactactggggccaagggaccccggtcaccgtgagctcaggaggtggcggctccggaggtggaggcagcggagggggcggatccgacatccagctgacccagagcccaagcagcctgagcgccagcgtgggtgacagagtgaccatcacctgtaagtccagtcaaagtgttttatacagttcaaatcagaagaactacttggcctggtaccagcagaagccaggtaaggctccaaagctgctgatctactgggcatccactagggaatctggtgtgccaagcagattcagcggtagcggtagcggtaccgacttcaccttcaccatcagcagcctccagccagaggacatcgccacctactactgccatcaatacctctcctcgtggacgttcggccaagggaccaagctggagatcaaagcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggccgccctggccggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattg

SEQ ID NO: 2 - DNA, 1200 bp, Homo sapiens
atggagacagacacactcctgctatgggtactgctgctctgggttccaggttccactggtgactctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggccgccctggccggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaaggtgacgcggcccagccggcccagaggacggactccattcccaactcacctgacaacgtccccctcaaaggaggaaattgctcagaagacctcttatgtatcctgaaaagagctccattcaagaagtcatgggcctacctccaagtggcaaagcatctgaacaaaaccaagttgtcttggaacaaagatggcattctccatggagtcagatatcaggatgggaatctggtgatccaattccctggtttgtacttcatcatttgccaactgcagtttcttgtacaatgcccaaataattctgtcgatctgaagttggagcttctcatcaacaagcatatcaaaaaacaggccctggtgacagtgtgtgagtctggaatgcaaacgaaacacgtataccagaatctctctcaattcttgctggattacctgcaggtcaacaccaccatatcagtcaatgtggatacattccagtacatagatacaagcacctttcctcttgagaatgtgttgtccatcttcttatacagtaattcagacgcggccgcagggccccatcatcatcatcatcattga

SEQ ID NO: 3 - DNA, 1491 bp, Mus sp.
atggagacagacacactcctgctatgggtactgctgctctgggttccaggttccactggtgacgcggcccagccggccatggcccaggtcaagctgcaggagtcagggactgaactggcaaagcctggggccgcagtgaagatgtcctgcaaggcttctggctacacctttactgactactggatgcactgggttaaacagaggcctggacagggtctggaatggattggatacattaatcctaacactgcttatactgactacaatcagaaattcaaggacaaggccacattgactgcagacaaatcctccagcacagcctacatgcaactgcgcagcctgacctctgaggattctgcagtctattactgtgcaaaaaagacaactcagactacgtgggggtttcctttttggggccaagggaccacggtcaccgtctcctcaggtggaggcggttcaggcggaggtggctctggcggtggcggatcggacattgtgctgacccagtctccaaaatccatggccatgtcagtcggagagagggtcaccttgagctgcaaggccagtgagaatgtggattcttttgtttcctggtatcaacagaaaccaggccagtctcctaaactgctgatatacggggcctccaaccggtacactggggtccccgatcgcttcgcaggcagtggatctggaagagatttcactctgaccatcagcagtgtgcaggctgaagaccttgcagattatcactgtggacagaattacaggtatccgctcacgttcggtgctggcaccaagctggaaatcaaacgggcggccgcatctggcggtggcggatcgctcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggccgccctggccggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 4 - DNA, 1473 bp, Mus sp.
atggagacagacacactcctgctatgggtactgctgctctgggttccaggttccactggtgacgcggcccagccggccatggccgaggtgcaactgcagcagtctggggctgaactggtgaagcctggggcttcagtgaagttgtcctgcaaggcttccggctacaccttcaccagccactggatgcactgggtgaagcagagggctggacaaggccttgagtggatcggagagtttaatcccagcaacggccgtactaactacaatgagaaattcaagagcaaggccacactgactgtagacaaatcctccagcacagcctacatgcaactcagcagcctgacatctgaggactctgcggtctattactgtgccagtcgggactatgattacgacggacggtactttgactactggggccaagggaccacggtcaccgtctcctcaggtggcggtggctcgggcggtggtgggtcgggtggtggcggatctgacatcgagctcacccagtctccagcaatcatgtctgcatctccaggggagaaggtcactatgacctgcagtgccagctcaagtgtaacttacatgtattggtaccagcagaagccaggatcctcccccagactcctgatttatgacacatccaacctggcttctggagtccctgttcgtttcagtggcagtgggtctgggacctcttactctctcacaatcagccgaatggaggctgaagatgctgccacttattactgccagcagtggagtagtcacatattcacgttcggctcggggacagaactcgagatcaaacgggcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggccgccctggccggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 5 - DNA, 843 bp, Homo sapiens
atggagacagacacactcctgctatgggtactgctgctctgggttccaggttccactggtgactctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggccgccctggccggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaaggtgacgcggcccagccggccaatagtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgcgcggccgcagggccccatcatcatcatcatcattga

SEQ ID NO: 6 - DNA, 1551 bp, Mus sp.
atggagacagacacactcctgctatgggtactgctgctctgggttccaggttccactggtgacgcggcccagccggccatggccgaggtgcaactgcagcagtctggggctgaactggtgaagcctggggcttcagtgaagttgtcctgcaaggcttccggctacaccttcaccagccactggatgcactgggtgaagcagagggctggacaaggccttgagtggatcggagagtttaatcccagcaacggccgtactaactacaatgagaaattcaagagcaaggccacactgactgtagacaaatcctccagcacagcctacatgcaactcagcagcctgacatctgaggactctgcggtctattactgtgccagtcgggactatgattacgacggacggtactttgactactggggccaagggaccacggtcaccgtctcctcaggtggcggtggctcgggcggtggtgggtcgggtggtggcggatctgacatcgagctcacccagtctccagcaatcatgtctgcatctccaggggagaaggtcactatgacctgcagtgccagctcaagtgtaacttacatgtattggtaccagcagaagccaggatcctcccccagactcctgatttatgacacatccaacctggcttctggagtccctgttcgtttcagtggcagtgggtctgggacctcttactctctcacaatcagccgaatggaggctgaagatgctgccacttattactgccagcagtggagtagtcacatattcacgttcggctcggggacagaactcgagatcaaacgggcggccgctagccgtcatcgccagccgcgcggcaatcgtgtccgacgctcacatatgcccttatcgtcaatcttctcgcgcattggggacccttctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggccgccctggccggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 7 - DNA, 1458 bp, Mus sp.
atggagacagacacactcctgctatgggtactgctgctctgggttccaggttccactggtgacgcggcccagccggccatggcccaggtgcagctggagcagtcggggggaggcttagtgaagcctggagggtccctgaaactctcctgtgcagcctctggattcactttcagtagctatgccatgtcttgggttcgccagactccggagaagaggctggagtgggtcgcaaccattaatagtggtggtagttacacctactattcggacagtgtgaagggtcgattcaccatctccagagacaatgccaggaataccctgtatctacaaatgagcagtctgaggtctgaggacacggccatgtattactgtgcaagaatctactatgctatggactactggggcgcaggaaccccggtcaccgtctcctcaggtggcggtggctcgggcggtggtgggtcgggtggcggcggatcagacattgtgatgacccagactccaaaatccatgtcaacattagtaggagacagggtcagcatcacctgcaaggccagtcaggatgtgagcactgctgtagcctggtatcaacagaaaccaggacaatctcctaaactactgatttactcggcatcctaccggtacactggagtccctgatcgcttcactggcagtggatctgggacggatttcactttcaccatcagcaatgtgcagtctgaagacttggcagagtatttctgtcagcaatataacagctatcctctgacgttcgccgcaggcaccaagctggagatcaaagcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 8 - DNA, 1107 bp, Homo sapiens
atggagacagacacactcctgctatgggtactgctgctctgggttccaggttccactggtgactctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggccgccctggccggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaaggtgacgcggcccagccggccggacaattcagagtgataggaccagggtatcccatccgggctttagttggggatgaagcagagctgccgtgccgcatctctcctgggaaaaatgccacgggcatggaggtgggttggtaccgttctcccttctcaagagtggttcacctctaccgaaatggcaaggaccaagatgcagagcaagcacctgaataccggggacgcacagagcttctgaaagagactatcagtgagggaaaggttacccttaggattcagaacgtgagattctcagatgaaggaggctacacctgcttcttcagagaccactcttaccaagaagaggcagcaatggagttgaaagtggaagatcccttctattgggtcaaccccggtgtggcggccgcagggcccgaacaaaaactcatctcagaagaggatctgaatagcgccgtcgaccatcatcatcatcatcattga

SEQ ID NO: 9 - DNA, 1428 bp, Homo sapiens
atggagacagacacactcctgctatgggtactgctgctctgggttccaggttccactggtgactctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggccgccctggccggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaaggtgacgcggcccagccggccgtgatgacccagtctccatcctccctgtctgcatctgtaggagacagagtcaccatcgcttgccgggcaagtcagaccattagcaactatttaaattggtatcagcagaaaccagggaaagcccctaagctcctgatctatggtgcatccagtttgcaaagtggggtcccatcaaggttcagtggcagtggatctgggacagatttcactctcaccatcagcagtctgcaacctgaagattttgcaacttactactgtcaacagagttacagtacccctccgacgtacacttttggccaggggaccaagctggagatcaaaggtggcggtggctcgggcggtggtgggtcgggtggcagcggatcatcggggggcgacttggtccagccgggggggtccctgagagtctcctgtgtagcctctggatttacatttaggacctatgtgatgaactgggtccgccaggctccaggaaaggggctggagtgggtggcccacataagtccagagggaactgaagaatactatgcggaccctgtgaagggccgatttaccgtctccagagacaacgcgaagaattcagtatttctgcaaatgaatagtctgagaggcgaggacacggctgtgtattattgcgcgagagtccgacgctatggtccctctacgctcagtccgttcacctggaaggacaatcactacgccatggacgtctggggccaagggacaacggtcaccgtctctccagcggccgcagggccccatcatcatcatcatcattga

SEQ ID NO: 10 - DNA, 774 bp, Human immunodeficiency virus
atggagacagacacactcctgctatgggtactgctgctctgggttccaggttccactggtgacgcggcccagccggcctacggtcgtaaaaaacgtcgtcagcgtcgtcgtagggcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggccgccctggccggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 11 - DNA, 1695 bp, Homo sapiens
atggagacagacacactcctgctatgggtactgctgctctgggttccaggttccactggtgacgcggcccagccggccgcacaggttctcagaggcactgtgactgacttccctggatttgatgagcgggctgatgcagaaactcttcggaaggctatgaaaggcttgggcacagatgaggagagcatcctgactctgttgacatcccgaagtaatgctcagcgccaggaaatctctgcagcttttaagactctgtttggcagggatcttctggatgacctgaaatcagaactaactggaaaatttgaaaaattaattgtggctctgatgaaaccctctcggctttatgatgcttatgaactgaaacatgccttgaagggagctggaacaaatgaaaaagtactgacagaaattattgcttcaaggacacctgaagaactgagagccatcaaacaagtttatgaagaagaatatggctcaagcctggaagatgacgtggtgggggacacttcagggtactaccagcggatgttggtggttctccttcaggctaacagagaccctgatgctggaattgatgaagctcaagttgaacaagatgctcaggctttatttcaggctggagaacttaaatgggggacagatgaagaaaagtttatcaccatctttggaacacgaagtgtgtctcatttgagaaaggtgtttgacaagtacatgactatatcaggatttcaaattgaggaaaccattgaccgcgagacttctggcaatttagagcaactactccttgctgttgtgaaatctattcgaagtatacctgcctaccttgcagagaccctctattatgctatgaagggagctgggacagatgatcataccctcatcagagtcatggtttccaggagtgagattgatctgtttaacatcaggaaggagtttaggaagaattttgccacctctctttattccatgattaagggagatacatctggggactataagaaagctcttctgctgctctgtggagaagatgacgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 12 - DNA, 1443 bp, Mus sp.
atggagacagacacactcctgctatgggtactgctgctctgggttccaggttccactggtgacgcggcccagccggcccagcagtctgggactgtgctggcaaggcctggggcttccgtgaagatgtcctgcaaggcttctggctacaggtttaccaactactggatgcactgggtaaaacagaggcctggacagggtctagaatggattggtgttatttatcctggaaatagtgatactagctacaaccagaagttcaagggcaaggccaaactgactgcagtcacatccgccagcactgcctacatggagctcagcagcctgacaaatgaggactctgcggtctattactgtacaagagagggagaaggctctgactactggggccaagggaccacggtcaccgtctcctcaggtggaggcggttcaggcggaggtggctctggcggtggcggatcgcaaattgttctcacccagtctccagcaaccatggctgcatctcccggggagaagatcactatcacctgcagtgccagctcaagtataagttccaattacttgcattggtatcagcagaagccaggattctcccctaaactcttgatttataggacttccaatctggcttctggagtcccagctcgcttcagtggcagtgggtctgggacctcttactctctcacaattggcaccatggaggctgaagatgttgccacttactactgccagcagggtagtagtataccgtacacgttcggaggggggaccaagctggagctgaaagcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 13 - DNA, 1467 bp, Homo sapiens
atggagacagacacactcctgctatgggtactgctgctctgggttccaggttccactggtgacgcggcccagccggccatggcccaggtgcagctgcaggagtcggggggaggcttggtacagcctggggggtccctgagactctcctgtgcagcctctggattcacctttagcaactctgccatgagctgggtccgccaggctccagggaaggggctggagtgggtctcaagtatcagtggtagtggcggtaacacatactccgctgactccgtgaagggccgattcaccatctccagagacaacgccaagaactcactgtatctgcaaatgaacagcctgagagccgaggacacggccgtgtattactgtgcgagagattggtacggtatggacgtctggggccaagggaccacggtcaccgtctcctcaggtggaggcggttcaggcggaggtggctctggcggtggcggatcgtcctatgtgctgactcaggaccctgctgtgtctgtggccttgggacagacagtcaggatcacatgccaaggagacagcctcagaggctattatgcaagctggtaccagcagaagccaggacaggcccctgtacttgtcatctatgctaaaaccaaccggccctcagggatcccagaccgattctctggctccagctcaggaaacacagcttccttgaccatcactggggctcaggcggaagatgaagctgactattactgtaactcccgggacaacagtggtacccatcttgaagtattcggcggagggaccaagctgaccgtcctaggtgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 14 - DNA, 1161 bp, Mus sp.
atggagacagacacactcctgctatgggtactgctgctctgggttccaggttccactggtgacgcggcccagccggccatggccgaggtgcaactgcagcagtctggggctgaactggtgaagcctggggcttcagtgaagttgtcctgcaaggcttccggctacaccttcaccagccactggatgcactgggtgaagcagagggctggacaaggccttgagtggatcggagagtttaatcccagcaacggccgtactaactacaatgagaaattcaagagcaaggccacactgactgtagacaaatcctccagcacagcctacatgcaactcagcagcctgacatctgaggactctgcggtctattactgtgccagtcgggactatgattacgacggacggtactttgactactggggccaagggaccacggtcaccgtctcctcaggtggcggtggctcgggcggtggtgggtcgggtggtggcggatctgacatcgagctcacccagtctccagcaatcatgtctgcatctccaggggagaaggtcactatgacctgcagtgccagctcaagtgtaacttacatgtattggtaccagcagaagccaggatcctcccccagactcctgatttatgacacatccaacctggcttctggagtccctgttcgtttcagtggcagtgggtctgggacctcttactctctcacaatcagccgaatggaggctgaagatgctgccacttattactgccagcagtggagtagtcacatattcacgttcggctcggggacagaactcgagatcaaacgggcggccgcactcgagtctagaatgagcactatcgaagaacgcgttaagaaaattatcggcgaacagctgggcgttaagcaggaagaagttaccaacaatgcttctttcgttgaagacctgggcgcggattctcttgacaccgttgagctggtaatggctctggaagaagagtttgatactgagattccggacgaagaagctgagaaaatcaccaccgttcaggctgccattgattacatcaacggccaccaggcggctgagcacgaatttcgaggagggcccgaacaaaaactcatctcagaagaggatctgaatagcgccgtcgaccatcatcatcatcatcattga

SEQ ID NO: 15 - DNA, 1179 bp, Mus sp.
atggagacagacacactcctgctatgggtactgctgctctgggttccaggttccactggtgacgcggcccagccggccatggcccaggtcaagctgcaggagtcagggactgaactggcaaagcctggggccgcagtgaagatgtcctgcaaggcttctggctacacctttactgactactggatgcactgggttaaacagaggcctggacagggtctggaatggattggatacattaatcctaacactgcttatactgactacaatcagaaattcaaggacaaggccacattgactgcagacaaatcctccagcacagcctacatgcaactgcgcagcctgacctctgaggattctgcagtctattactgtgcaaaaaagacaactcagactacgtgggggtttcctttttggggccaagggaccacggtcaccgtctcctcaggtggaggcggttcaggcggaggtggctctggcggtggcggatcggacattgtgctgacccagtctccaaaatccatggccatgtcagtcggagagagggtcaccttgagctgcaaggccagtgagaatgtggattcttttgtttcctggtatcaacagaaaccaggccagtctcctaaactgctgatatacggggcctccaaccggtacactggggtccccgatcgcttcgcaggcagtggatctggaagagatttcactctgaccatcagcagtgtgcaggctgaagaccttgcagattatcactgtggacagaattacaggtatccgctcacgttcggtgctggcaccaagctggaaatcaaacgggcggccgcatctggcggtggcggatcgctcgagtctagaatgagcactatcgaagaacgcgttaagaaaattatcggcgaacagctgggcgttaagcaggaagaagttaccaacaatgcttctttcgttgaagacctgggcgcggattctcttgacaccgttgagctggtaatggctctggaagaagagtttgatactgagattccggacgaagaagctgagaaaatcaccaccgttcaggctgccattgattacatcaacggccaccaggcggctgagcacgaatttcgaggagggcccgaacaaaaactcatctcagaagaggatctgaatagcgccgtcgaccatcatcatcatcatcattga

SEQ ID NO: 16 - DNA, 1473 bp, Mus sp.
atggagacagacacactcctgctatgggtactgctgctctgggttccaggttccactggtgacgcggcccagccggccatggccgaggtgcaactgcagcagtctggggctgaactggtgaagcctggggcttcagtgaagttgtcctgcaaggcttccggctacaccttcaccagccactggatgcactgggtgaagcagagggctggacaaggccttgagtggatcggagagtttaatcccagcaacggccgtactaactacaatgagaaattcaagagcaaggccacactgactgtagacaaatcctccagcacagcctacatgcaactcagcagcctgacatctgaggactctgcggtctattactgtgccagtcgggactatgattacgacggacggtactttgactactggggccaagggaccacggtcaccgtctcctcaggtggcggtggctcgggcggtggtgggtcgggtggtggcggatctgacatcgagctcacccagtctccagcaatcatgtctgcatctccaggggagaaggtcactatgacctgcagtgccagctcaagtgtaacttacatgtattggtaccagcagaagccaggatcctcccccagactcctgatttatgacacatccaacctggcttctggagtccctgttcgtttcagtggcagtgggtctgggacctcttactctctcacaatcagccgaatggaggctgaagatgctgccacttattactgccagcagtggagtagtcacatattcacgttcggctcggggacagaactcgagatcaaacgggcggccgcactcgagtctagaatggacaaagactgcgaaatgaagcgcaccaccctggatagccctctgggcaagctggaactgtctgggtgcgaacagggcctgcacgagatcatcttcctgggcaaaggaacatctgccgccgacgccgtggaagtgcctgccccagccgccgtgctgggcggaccagagccactgatccaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagcgagagccacctggccgccctggtgggcaatcccgccgccaccgccgccgtgaacaccgccctggacggaaatcccgtgcccattctgatcccctgccaccgggtggtgcagggcgacagcgacgtggggccctacctgggcgggctcgccgtgaaagagtggctgctggcccacgagggccacagactgggcaagcctgggctgggtgctgagcacgaatttcgaggagggcccgaacaaaaactcatctcagaagaggatctgaatagcgccgtcgaccatcatcatcatcatcattga

SEQ ID NO: 17 - DNA, 1491 bp, Mus sp.
atggagacagacacactcctgctatgggtactgctgctctgggttccaggttccactggtgacgcggcccagccggccatggcccaggtcaagctgcaggagtcagggactgaactggcaaagcctggggccgcagtgaagatgtcctgcaaggcttctggctacacctttactgactactggatgcactgggttaaacagaggcctggacagggtctggaatggattggatacattaatcctaacactgcttatactgactacaatcagaaattcaaggacaaggccacattgactgcagacaaatcctccagcacagcctacatgcaactgcgcagcctgacctctgaggattctgcagtctattactgtgcaaaaaagacaactcagactacgtgggggtttcctttttggggccaagggaccacggtcaccgtctcctcaggtggaggcggttcaggcggaggtggctctggcggtggcggatcggacattgtgctgacccagtctccaaaatccatggccatgtcagtcggagagagggtcaccttgagctgcaaggccagtgagaatgtggattcttttgtttcctggtatcaacagaaaccaggccagtctcctaaactgctgatatacggggcctccaaccggtacactggggtccccgatcgcttcgcaggcagtggatctggaagagatttcactctgaccatcagcagtgtgcaggctgaagaccttgcagattatcactgtggacagaattacaggtatccgctcacgttcggtgctggcaccaagctggaaatcaaacgggcggccgcatctggcggtggcggatcgctcgagtctagaatggacaaagactgcgaaatgaagcgcaccaccctggatagccctctgggcaagctggaactgtctgggtgcgaacagggcctgcacgagatcatcttcctgggcaaaggaacatctgccgccgacgccgtggaagtgcctgccccagccgccgtgctgggcggaccagagccactgatccaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagcgagagccacctggccgccctggtgggcaatcccgccgccaccgccgccgtgaacaccgccctggacggaaatcccgtgcccattctgatcccctgccaccgggtggtgcagggcgacagcgacgtggggccctacctgggcgggctcgccgtgaaagagtggctgctggcccacgagggccacagactgggcaagcctgggctgggtgctgagcacgaatttcgaggagggcccgaacaaaaactcatctcagaagaggatctgaatagcgccgtcgaccatcatcatcatcatcattga

SEQ ID NO: 18 - DNA, 1182 bp, Homo sapiens
atggagacagacacactcctgctatgggtactgctgctctgggttccaggttccactggtgacgcggcccagccggccccggtcatgaggctgttcccttgcttcctgcagctcctggccgggctggcgctgcctgctgtgcccccccagcagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggtgcggcgatgctgttccccggagggcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 19 - DNA, 1194 bp, Homo sapiens
atggagacagacacactcctgctatgggtactgctgctctgggttccaggttccactggtgacgcggcccagccggcctacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 20 - DNA, 1518 bp, Homo sapiens
atggagacagacacactcctgctatgggtactgctgctctgggttccaggttccactggtgacgcggcccagccggccatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactcgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 21 - DNA, 1308 bp, Homo sapiens
atggagacagacacactcctgctatgggtactgctgctctgggttccaggttccactggtgacgcggcccagccggccaactttctgctgtcttgggtgcattggagcctcgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcgggcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 22 - DNA, 1542 bp, Homo sapiens
atggagacagacacactcctgctatgggtactgctgctctgggttccaggttccactggtgacgcggcccagccggccgcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttccgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 23 - DNA, 1191 bp, Homo sapiens
atggagacagacacactcctgctatgggtactgctgctctgggttccaggttccactggtgacgcggcccagccggccagccgcctgcccgtcctgctcctgctccaactcctggtccgccccggactccaagctcccatgacccagacaacgtccttgaagacaagctgggttaactgctctaacatgatcgatgaaattataacacacttaaagcagccacctttgcctttgctggacttcaacagcctcaatggggaagaccaagacattctgatggaaaataaccttcgaaggccaaacctggaggcattcaacagggctgtcaagagtttacagaacgcatcagcaattgagagcattcttaaaaatctcctgccatgtctgcccctggccacggccgcacccacgcgacatccaatccatatcaaggacggtgactggaatgaattccggaggaaactgacgttctatctgaaaacccttgagaatgcgcaggctcaacagacgactttgagcctcgcgatctttgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 24 - DNA, 1194 bp, Homo sapiens
atggagacagacacactcctgctatgggtactgctgctctgggttccaggttccactggtgacgcggcccagccggccggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagcgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 25 - DNA, 1080 bp, Homo sapiens
atggagacagacacactcctgctatgggtactgctgctctgggttccaggttccactggtgacgcggcccagccggcccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 26 - DNA, 1371 bp, Homo sapiens
atggagacagacacactcctgctatgggtactgctgctctgggttccaggttccactggtgacgcggcccagccggccaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatggcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 27 - DNA, 1194 bp, Homo sapiens
atggagacagacacactcctgctatgggtactgctgctctgggttccaggttccactggtgacgcggcccagccggccgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacacgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 28 - DNA, 975 bp, Homo sapiens
atggagacagacacactcctgctatgggtactgctgctctgggttccaggttccactggtgacgcggcccagccggccgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcagcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 29 - DNA, 1115 bp, Homo sapiens
atggagacagacacactcctgctatgggtactgctgctctgggttccaggttccactggtgacgcggcccagccggccaggggtgtccaaccttggcggggatcctggacatcaacttcctcatcaacaagatgcaggaagatccagcttccaagtgccactgcagtgctaatgtgaccagttgtctctgtttgggcattccctctgacaactgcaccagaccatgcttcagtgagagactgtctcagatgaccaataccaccatgcaaacaagatacccactgattttcagtcgggtgaaaaaatcagttgaagtactaaagaacaacaagtgtccatatttttcctgtgaacagccatgcaaccaaaccacggcaggcaacgcgctgacatttctgaagagtcttctggaaattttccagaaagaaaagatgagagggatgagaggcaagatagcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 30 - DNA, 1218 bp, Homo sapiens
atggagacagacacactcctgctatgggtactgctgctctgggttccaggttccactggtgacgcggcccagccggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaacgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 31 - DNA, 1272 bp, Homo sapiens
atggagacagacacactcctgctatgggtactgctgctctgggttccaggttccactggtgacgcggcccagccggcccctgggccaccacctggcccccctcgagtttccccagaccctcgggccgagctggacagcaccgtgctcctgacccgctctctcctggcggacacgcggcagctggctgcacagctgagggacaaattcccagctgacggggaccacaacctggattccctgcccaccctggccatgagtgcgggggcactgggagctctacagctcccaggtgtgctgacaaggctgcgagcggacctactgtcctacctgcggcacgtgcagtggctgcgccgggcaggtggctcttccctgaagaccctggagcccgagctgggcaccctgcaggcccgactggaccggctgctgcgccggctgcagctcctgatgtcccgcctggccctgccccagccacccccggacccgccggcgcccccgctggcgcccccctcctcagcctgggggggcatcagggccgcccacgccatcctgggggggctgcacctgacacttgactgggccgtgaggggactgctgctgctgaagactcggctggcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 32 - DNA, 1329 bp, Homo sapiens
atggagacagacacactcctgctatgggtactgctgctctgggttccaggttccactggtgacgcggcccagccggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 33 - DNA, 1074 bp, Homo sapiens
atggagacagacacactcctgctatgggtactgctgctctgggttccaggttccactggtgacgcggcccagccggccggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacagttcaacgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 34 - DNA, 1080 bp, Homo sapiens
atggagacagacacactcctgctatgggtactgctgctctgggttccaggttccactggtgacgcggcccagccggccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggagaaaaaaaatattaaagaatttttgcagagttttgtacatattgtccaaatgttcatcaacacttctgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 35 - DNA, 2097 bp, Homo sapiens
atggagacagacacactcctgctatgggtactgctgctctgggttccaggttccactggtgacgcggcccagccggccgagtcgcacagccgcgctggaaagagcagaaaatctgcaaaatttcggtccatctccaggtccctgatgctctgtaatgctaagaccagtgatgatggctctagccctgatgagaaatatcctgatccctttgagatttccttggcccagggcaaggagggaattttccactcatctgtgcagctggcagacacatcggaggctgggcccagcagtgttcctgatctagcactggcctcggaggctgctcaactccaagcagctgggaatgatcgaggcaagacctgtaggaggatattcttcatgaaggaatcttccacagcttcctctcgagaaaagcctggaaaactagaagcacaaagtagtaacttcctgtttcctaaagcctgccaccaaagggcacgcagcaactcaaccagtgttaatccctattgcacaagagaaattgattttccaatgaccaagaaatctgcagcgcccacggacaggcagccttactctctctgcagtaacaggaagtccctctctcaacaattggactgtccagcaggaaaggctgcgggaacttcgagaccaacacggtccctgagcacagctcagctcgtgcagccatctgggggcctccaggcttcagtcatctccaacatcgtgctgatgaagggccaggctaagggtctgggcttcagcatcgttgggggaaaagacagcatttatggccccattgggatttacgtcaaaaccatttttgcagggggagcagcagcagccgatggaaggctacaggaaggtgatgaaattctggagctcaatggtgaatcaatggctggactaacacatcaggatgctttgcagaagttcaagcaagccaaaaaggggctcctcaccctcaccgtgagaacccgcctgacggcgcctccttccctgtgcagccacctgtctcccccactgtgccgctccctgagctccagcacttgtatcaccaaggacagcagctccttcgccttggaaagcccctcggctcccatcagcaccgccaagcccaattacagaatcatggtggaggtttctctgcagaaagaggccggcgtgggcctgggcatcggcctgtgcagcgttccctacttccaatgcatctctggcattttcgtccacacgctgtcaccaggatccgtggcgcacctggacggacgtctccggtgtggggacgagattgtggaaatcagtgattcccctgtgcactgcctgacgctcaatgaagtctacacgatcctgagtcactgtgatcccggtccagtctccatcattgttagccgacatccagacccacagaaggaagaaatggaggctcaggaggttaaggcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 36 - DNA, 1203 bp, Homo sapiens
atggagacagacacactcctgctatgggtactgctgctctgggttccaggttccactggtgacgcggcccagccggccatgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggccgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 37 - DNA, 1208 bp, Homo sapiens
atggagacagacacactcctgctatgggtactgctgctctgggttccaggttccactggtgacgcggcccagccggccactttggcaagcttgaatctaaattatcagtcataagaaatttgaatgaccaagttctcttcattgaccaaggaaatcggcctctatttgaagatatgactgattctgactgtagagataatgcaccccggaccatatttattataagtatgtataaagatagccagcctagaggtatggctgtaactatctctgtgaagtgtgagaaaatttcaactctctcctgtgagaacaaaattatttcctttaaggaaatgaatcctcctgataacatcaaggatacaaaaagtgacatcatattctttcagagaagtgtcccaggacatgataataagatgcaatttgaatcttcatcatacgaaggatactttctagcttgtgaaaaagagagagacctttttaaactcattttgaaaaaagaggatgaattgggggatagatctataatgttcactgttcaaaacgaagacgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 38 - DNA, 1215 bp, Homo sapiens
atggagacagacacactcctgctatgggtactgctgctctgggttccaggttccactggtgacgcggcccagccggcctcagtagacaaccacggtctcaggagatgtctgatttccacagacatgcaccatatagaagagagtttccaagaaatcaaaagagccatccaagctaaggacaccttcccaaatgtcactatcctgtccacattggagactctgcagatcattaagcccttagatgtgtgctgcgtgaccaagaacctcctggcgttctacgtggacagggtgttcaaggatcatcaggagccaaaccccaaaatcttgagaaaaatcagcagcattgccaactctttcctctacatgcagaaaactctgcggcaatgtcaggaacagaggcagtgtcactgcaggcaggaagccaccaatgccaccagagtcatccatgacaactatgatcagctggaggtccacgctgctgccattaaatccctgggagagctcgacgtctttctagcctggattaataagaatcatgaagtaatgttctcagctgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 39 - DNA, 1266 bp, Homo sapiens
atggagacagacacactcctgctatgggtactgctgctctgggttccaggttccactggtgacgcggcccagccggccatgaaagcctctagtcttgccttcagccttctctctgctgcgttttatctcctatggactccttccactggactgaagacactcaatttgggaagctgtgtgatcgccacaaaccttcaggaaatacgaaatggattttctgagatacggggcagtgtgcaagccaaagatggaaacattgacatcagaatcttaaggaggactgagtctttgcaagacacaaagcctgcgaatcgatgctgcctcctgcgccatttgctaagactctatctggacagggtatttaaaaactaccagacccctgaccattatactctccggaagatcagcagcctcgccaattcctttcttaccatcaagaaggacctccggctctgtcatgcccacatgacatgccattgtggggaggaagcaatgaagaaatacagccagattctgagtcactttgaaaagctggaacctcaggcagcagttgtgaaggctttgggggaactagacattcttctgcaatggatggaggagacagaagcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 40 - DNA, 1221 bp, Homo sapiens
atggagacagacacactcctgctatgggtactgctgctctgggttccaggttccactggtgacgcggcccagccggccagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattccgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 41 - DNA, 1272 bp, Homo sapiens
atggagacagacacactcctgctatgggtactgctgctctgggttccaggttccactggtgacgcggcccagccggccgccgccctgcagaaatctgtgagctctttccttatggggaccctggccaccagctgcctccttctcttggccctcttggtacagggaggagcagctgcgcccatcagctcccactgcaggcttgacaagtccaacttccagcagccctatatcaccaaccgcaccttcatgctggctaaggaggctagcttggctgataacaacacagacgttcgtctcattggggagaaactgttccacggagtcagtatgagtgagcgctgctatctgatgaagcaggtgctgaacttcacccttgaagaagtgctgttccctcaatctgataggttccagccttatatgcaggaggtggtgcccttcctggccaggctcagcaacaggctaagcacatgtcatattgaaggtgatgacctgcatatccagaggaatgtgcaaaagctgaaggacacagtgaaaaagcttggagagagtggagagatcaaagcaattggagaactggatttgctgtttatgtctctgagaaatgcctgcattgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 42 - DNA, 1248 bp, Homo sapiens
atggagacagacacactcctgctatgggtactgctgctctgggttccaggttccactggtgacgcggcccagccggccagagctgtgcctgggggcagcagccctgcctggactcagtgccagcagctttcacagaagctctgcacactggcctggagtgcacatccactagtgggacacatggatctaagagaagagggagatgaagagactacaaatgatgttccccatatccagtgtggagatggctgtgacccccaaggactcagggacaacagtcagttctgcttgcaaaggatccaccagggtctgattttttatgagaagctgctaggatcggatattttcacaggggagccttctctgctccctgatagccctgtgggccagcttcatgcctccctactgggcctcagccaactcctgcagcctgagggtcaccactgggagactcagcagattccaagcctcagtcccagccagccatggcagcgtctccttctccgcttcaaaatccttcgcagcctccaggcctttgtggctgtagccgcccgggtctttgcccatggagcagcaaccctgagtcccgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 43 - DNA, 1212 bp, Homo sapiens
atggagacagacacactcctgctatgggtactgctgctctgggttccaggttccactggtgacgcggcccagccggccgcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctcgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 44 - DNA, 1173 bp, Homo sapiens
atggagacagacacactcctgctatgggtactgctgctctgggttccaggttccactggtgacgcggcccagccggcctacagccactggcccagctgctgccccagcaaagggcaggacacctctgaggagctgctgaggtggagcactgtgcctgtgcctcccctagagcctgctaggcccaaccgccacccagagtcctgtagggccagtgaagatggacccctcaacagcagggccatctccccctggagatatgagttggacagagacttgaaccggctcccccaggacctgtaccacgcccgttgcctgtgcccgcactgcgtcagcctacagacaggctcccacatggacccccggggcaactcggagctgctctaccacaaccagactgtcttctaccggcggccatgccatggcgagaagggcacccacaagggctactgcctggagcgcaggctgtaccgtgtttccttagcttgtgtgtgtgtgcggccccgtgtgatgggcgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 45 - DNA, 1188 bp, Homo sapiens
atggagacagacacactcctgctatgggtactgctgctctgggttccaggttccactggtgacgcggcccagccggccaagcacaagcaatcttccttcaccaaaagttgttacccaaggggaacattgtcccaagctgttgacgctctctatatcaaagcagcatggctcaaagcaacgattccagaagaccgcataaaaaatatacgattattaaaaaagaaaacaaaaaagcagtttatgaaaaactgtcaatttcaagaacagcttctgtccttcttcatggaagacgtttttggtcaactgcaattgcaaggctgcaagaaaatacgctttgtggaggactttcatagccttaggcagaaattgagccactgtatttcctgtgcttcatcagctagagagatgaaatccattaccaggatgaaaagaatattttataggattggaaacaaaggaatctacaaagccatcagtgaactggatattcttctttcctggattaaaaaattattggaaagcagtcaggcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 46 - DNA, 1464 bp, Homo sapiens
atggagacagacacactcctgctatgggtactgctgctctgggttccaggttccactggtgacgcggcccagccggccggccagacggcaggcgaccttggctggcggctcagcctgttgctgcttcccttgctcctggttcaagctggtgtctggggattcccaaggcccccagggaggccccagctgagcctgcaggagctgcggagggagttcacagtcagcctgcatctcgccaggaagctgctcgccgaggttcggggccaggcccaccgctttgcggaatctcacctgccaggagtgaacctgtacctcctgcccctgggagagcagctccctgatgtttccctgaccttccaggcctggcgccgcctctctgacccggagcgtctctgcttcatctccaccacgcttcagcccttccatgccctgctgggagggctggggacccagggccgctggaccaacatggagaggatgcagctgtgggccatgaggctggacctccgcgatctgcagcggcacctccgcttccaggtgctggctgcaggattcaacctcccggaggaggaggaggaggaagaggaggaggaggaggaggagaggaaggggctgctcccaggggcactgggcagcgccttacagggcccggcccaggtgtcctggccccagctcctctccacctaccgcctgctgcactccttggagctcgtcttatctcgggccgtgcgggagttgctgctgctgtccaaggctgggcactcagtctggcccttggggttcccaacattgagcccccagcccgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 47 - DNA, 1335 bp, Homo sapiens
atggagacagacacactcctgctatgggtactgctgctctgggttccaggttccactggtgacgcggcccagccggccaaactagacatgactggggactgcacgccagtgctggtgctgatggccgcagtgctgaccgtgactggagcagttcctgtcgccaggctccacggggctctcccggatgcaaggggctgccacatagcccagttcaagtccctgtctccacaggagctgcaggcctttaagagggccaaagatgccttagaagagtcgcttctgctgaaggactgcaggtgccactcccgcctcttccccaggacctgggacctgaggcagctgcaggtgagggagcgccccatggctttggaggctgagctggccctgacgctgaaggttctggaggccaccgctgacactgacccagccctggtggacgtcttggaccagccccttcacaccctgcaccatatcctctcccagttccgggcctgtatccagcctcagcccacggcagggcccaggacccggggccgcctccaccattggctgtaccggctccaggaggccccaaaaaaggagtcccctggctgcctcgaggcctctgtcaccttcaacctcttccgcctcctcacgcgagacctgaattgtgttgccagtggggacctgtgtgtcgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 48 - DNA, 1323 bp, Homo sapiens
atggagacagacacactcctgctatgggtactgctgctctgggttccaggttccactggtgacgcggcccagccggccaccggggactgcatgccagtgctggtgctgatggccgcagtgctgaccgtgactggagcagttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagtgccgctcccgcctcttccccaggacctgggacctgaggcagctgcaggtgagggagcgccccgtggctttggaggctgagctggccctgacgctgaaggttctggaggccaccgctgacactgacccagccctgggggatgtcttggaccagccccttcacaccctgcaccatatcctctcccagctccgggcctgtatccagcctcagcccacggcagggcccaggacccggggccgcctccaccattggctgcaccggctccaggaggccccaaaaaaggagtcccctggctgcctcgaggcctctgtcaccttcaacctcttccgcctcctcacgcgagacctgaattgtgttgccagcggggacctgtgtgtcgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 49 - DNA, 1250 bp, Homo sapiens
atggagacagacacactcctgctatgggtactgctgctctgggttccaggttccactggtgacgcggcccagccggccctgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgtgtctgagaacgtcaacccaccctgagtccaccgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 50 - DNA, 1161 bp, Homo sapiens
atggagacagacacactcctgctatgggtactgctgctctgggttccaggttccactggtgacgcggcccagccggcctcccacacgttgcccgtccgtttactacgaccaagtgatgatgtacagaaaatagtcgaggaattacagtccctctcgaagatgcttttgaaagatgtggaggaagagaagggcgtgctcgtgtcccagaattacacgctgccgtgtctcagccctgacgcccagccgccaaacaacatccacagcccagccatccgggcatatctcaagacaatcagacagctagacaacaaatctgttattgatgagatcatagagcacctcgacaaactcatatttcaagatgcaccagaaacaaacatttctgtgccaacagacacccatgaatgtaaacgcttcatcctgactatttctcaacagttttcagagtgcatggacctcgcactaaaatcattgacctctggagcccaacaggccaccactgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 51 - DNA, 1047 bp, Homo sapiens
atggagacagacacactcctgctatgggtactgctgctctgggttccaggttccactggtgacgcggcccagccggccaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacagcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 52 - DNA, 969 bp, Homo sapiens
atggagacagacacactcctgctatgggtactgctgctctgggttccaggttccactggtgacgcggcccagccggccgtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctcctgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 53 - DNA, 1554 bp, Homo sapiens
atggagacagacacactcctgctatgggtactgctgctctgggttccaggttccactggtgacgcggcccagccggcctctgggagtcagagcgaggtggctccatccccgcagagtccgcggagccccgagatgggacgggacttgcggcccgggtcccgcgtgctcctgctcctgcttctgctcctgctggtgtacctgactcagccaggcaatggcaacgagggcagcgtcactggaagttgttattgtggtaaaagaatttcttccgactccccgccatcggttcagttcatgaatcgtctccggaaacacctgagagcttaccatcggtgtctatactacacgaggttccagctcctttcctggagcgtgtgtgggggcaacaaggacccatgggttcaggaattgatgagctgtcttgatctcaaagaatgtggacatgcttactcggggattgtggcccaccagaagcatttacttcctaccagccccccaatttctcaggcctcagagggggcatcttcagatatccacacccctgcccagatgctcctgtccaccttgcagtccactcagcgccccaccctcccagtaggatcactgtcctcggacaaagagctcactcgtcccaatgaaaccaccattcacactgcgggccacagtctggcagctgggcctgaggctggggagaaccagaagcagccggaaaaaaatgctggtcccacagccaggacatcagccacagtgccagtcctgtgcctcctggccatcatcttcatcctcaccgcagccctttcctatgtgctgtgcaagaggaggagggggcagtcaccgcagtcctctccagatctgccggttcattatatacctgtggcacctgactctaataccgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 54 - DNA, 957 bp, Homo sapiens
atggagacagacacactcctgctatgggtactgctgctctgggttccaggttccactggtgacgcggcccagccggccttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaattttgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 55 - DNA, 999 bp, Homo sapiens
atggagacagacacactcctgctatgggtactgctgctctgggttccaggttccactggtgacgcggcccagccggccgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattcccgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 56 - DNA, 960 bp, Homo sapiens
atggagacagacacactcctgctatgggtactgctgctctgggttccaggttccactggtgacgcggcccagccggccgggccagcttctgtcccaaccacctgctgctttaacctggccaataggaagataccccttcagcgactagagagctacaggagaatcaccagtggcaaatgtccccagaaagctgtgatcttcaagaccaaactggccaaggatatctgtgccgaccccaagaagaagtgggtgcaggattccatgaagtatctggaccaaaaatctccaactccaaagccagcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 57 - DNA, 960 bp, Homo sapiens
atggagacagacacactcctgctatgggtactgctgctctgggttccaggttccactggtgacgcggcccagccggccaccaagactgaatcctcctcacggggaccttaccacccctcagagtgctgcttcacctacactacctacaagatcccgcgtcagcggattatggattactatgagaccaacagccagtgctccaagcccggaattgtcttcatcaccaaaaggggccattccgtctgtaccaaccccagtgacaagtgggtccaggactatatcaaggacatgaaggagaacgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 58 - DNA, 1029 bp, Homo sapiens
atggagacagacacactcctgctatgggtactgctgctctgggttccaggttccactggtgacgcggcccagccggcccagccaaaagttcctgagtgggtgaacaccccatccacctgctgcctgaagtattatgagaaagtgttgccaaggagactagtggtgggatacagaaaggccctcaactgtcacctgccagcaatcatcttcgtcaccaagaggaaccgagaagtctgcaccaaccccaatgacgactgggtccaagagtacatcaaggatcccaacctacctttgctgcctaccaggaacttgtccacggttaaaattattacagcaaagaatggtcaaccccagctcctcaactcccaggcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 59 - DNA, 945 bp, Homo sapiens
atggagacagacacactcctgctatgggtactgctgctctgggttccaggttccactggtgacgcggcccagccggccgcacaagttggtaccaacaaagagctctgctgcctcgtctatacctcctggcagattccacaaaagttcatagttgactattctgaaaccagcccccagtgccccaagccaggtgtcatcctcctaaccaagagaggccggcagatctgtgctgaccccaataagaagtgggtccagaaatacatcagcgacctgaagctgaatgccgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 60 - DNA, 1002 bp, Homo sapiens
atggagacagacacactcctgctatgggtactgctgctctgggttccaggttccactggtgacgcggcccagccggccttcctactgccacccagcactgcctgctgtactcagctctaccgaaagccactctcagacaagctactgaggaaggtcatccaggtggaactgcaggaggctgacggggactgtcacctccaggctttcgtgcttcacctggctcaacgcagcatctgcatccacccccagaaccccagcctgtcacagtggtttgagcaccaagagagaaagctccatgggactctgcccaagctgaattttgggatgctaaggaaaatgggcgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 61 - DNA, 1083 bp, Homo sapiens
atggagacagacacactcctgctatgggtactgctgctctgggttccaggttccactggtgacgcggcccagccggccgtctgtgcggccctacatgcctcagaagccatacttcccattgcctccagctgttgcacggaggtttcacatcatatttccagaaggctcctggaaagagtgaatatgtgtcgcatccagagagctgatggggattgtgacttggctgctgtcatccttcatgtcaagcgcagaagaatctgtgtcagcccgcacaaccatactgttaagcagtggatgaaagtgcaagctgccaagaaaaatggtaaaggaaatgtttgccacaggaagaaacaccatggcaagaggaacagtaacagggcacatcaggggaaacacgaaacatacggccataaaactccttatgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 62 - DNA, 1017 bp, Homo sapiens
atggagacagacacactcctgctatgggtactgctgctctgggttccaggttccactggtgacgcggcccagccggccgtagggagtgaagtctcagataagaggacctgtgtgagcctcactacccagcgactgccggttagcagaatcaagacctacaccatcacggaaggctccttgagagcagtaatttttattaccaaacgtggcctaaaagtctgtgctgatccacaagccacatgggtgagagacgtggtcaggagcatggacaggaaatccaacaccagaaataacatgatccagaccaagccaacaggaacccagcaatcgaccaatacagctgtgactctgactggcgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 63 - DNA, 1857 bp, Homo sapiens
atggagacagacacactcctgctatgggtactgctgctctgggttccaggttccactggtgacgcggcccagccggcccagcaccacggtgtgacgaaatgcaacatcacgtgcagcaagatgacatcaaagatacctgtagctttgctcatccactatcaacagaaccaggcatcatgcggcaaacgcgcaatcatcttggagacgagacagcacaggctgttctgtgccgacccgaaggagcaatgggtcaaggacgcgatgcagcatctggaccgccaggctgctgccctaactcgaaatggcggcaccttcgagaagcagatcggcgaggtgaagcccaggaccacccctgccgccgggggaatggacgagtctgtggtcctggagcccgaagccacaggcgaaagcagtagcctggagccgactccttcttcccaggaagcacagagggccctggggacctccccagagctgccgacgggcgtgactggttcctcagggaccaggctccccccgacgccaaaggctcaggatggagggcctgtgggcacggagcttttccgagtgcctcccgtctccactgccgccacgtggcagagttctgctccccaccaacctgggcccagcctctgggctgaggcaaagacctctgaggccccgtccacccaggacccctccacccaggcctccactgcgtcctccccagccccagaggagaatgctccgtctgaaggccagcgtgtgtggggtcagggacagagccccaggccagagaactctctggagcgggaggagatgggtcccgtgccagcgcacacggatgccttccaggactgggggcctggcagcatggcccacgtctctgtggtccctgtctcctcagaagggacccccagcagggagccagtggcttcaggcagctggacccctaaggctgaggaacccatccatgccaccatggacccccagaggctgggcgtccttatcactcctgtccctgacgcccaggctgccacccggaggcaggcggtggggctgctggccttccttggcctcctcttctgcctgggggtggccatgttcacctaccagagcctccagggctgccctcgaaagatggcaggagagatggcggagggccttcgctacatcccccggagctgtggtagtaattcatatgtcctggtgcccgtggcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 64 - DNA, 1074 bp, Homo sapiens
atggagacagacacactcctgctatgggtactgctgctctgggttccaggttccactggtgacgcggcccagccggccgccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagcgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 65 - DNA, 1269 bp, Homo sapiens
atggagacagacacactcctgctatgggtactgctgctctgggttccaggttccactggtgacgcggcccagccggccacccccctgggccctgccagctccctgccccagagcttcctgctcaagtgcttagagcaagtgaggaagatccagggcgatggcgcagcgctccaggagaagctggtgagtgagtgtgccacctacaagctgtgccaccccgaggagctggtgctgctcggacactctctgggcatcccctgggctcccctgagcagctgccccagccaggccctgcagctggcaggctgcttgagccaactccatagcggccttttcctctaccaggggctcctgcaggccctggaagggatctcccccgagttgggtcccaccttggacacactgcagctggacgtcgccgactttgccaccaccatctggcagcagatggaagaactgggaatggcccctgccctgcagcccacccagggtgccatgccggccttcgcctctgctttccagcgccgggcaggaggggtcctggttgcctcccatctgcagagcttcctggaggtgtcgtaccgcgttctacgccaccttgcccagcccgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 66 - DNA, 1407 bp, Homo sapiens
atggagacagacacactcctgctatgggtactgctgctctgggttccaggttccactggtgacgcggcccagccggccgaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagccgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 67 - DNA, 2814 bp, Homo sapiens
atggagacagacacactcctgctatgggtactgctgctctgggttccaggttccactggtgacgcggcccagccggcccaaaggaaaagaagaaatacaattcatgaattcaaaaaatcagcaaagactaccctaatcaaaatagatccagcactgaagataaaaaccaaaaaagtgaatactgcagaccaatgtgctaatagatgtactaggaataaaggacttccattcacttgcaaggcttttgtttttgataaagcaagaaaacaatgcctctggttccccttcaatagcatgtcaagtggagtgaaaaaagaatttggccatgaatttgacctctatgaaaacaaagactacattagaaactgcatcattggtaaaggacgcagctacaagggaacagtatctatcactaagagtggcatcaaatgtcagccctggagttccatgataccacacgaacacagctatcggggtaaagacctacaggaaaactactgtcgaaatcctcgaggggaagaagggggaccctggtgtttcacaagcaatccagaggtacgctacgaagtctgtgacattcctcagtgttcagaagttgaatgcatgacctgcaatggggagagttatcgaggtctcatggatcatacagaatcaggcaagatttgtcagcgctgggatcatcagacaccacaccggcacaaattcttgcctgaaagatatcccgacaagggctttgatgataattattgccgcaatcccgatggccagccgaggccatggtgctatactcttgaccctcacacccgctgggagtactgtgcaattaaaacatgcgctgacaatactatgaatgacactgatgttcctttggaaacaactgaatgcatccaaggtcaaggagaaggctacaggggcactgtcaataccatttggaatggaattccatgtcagcgttgggattctcagtatcctcacgagcatgacatgactcctgaaaatttcaagtgcaaggacctacgagaaaattactgccgaaatccagatgggtctgaatcaccctggtgttttaccactgatccaaacatccgagttggctactgctcccaaattccaaactgtgatatgtcacatggacaagattgttatcgtgggaatggcaaaaattatatgggcaacttatcccaaacaagatctggactaacatgttcaatgtgggacaagaacatggaagacttacatcgtcatatcttctgggaaccagatgcaagtaagctgaatgagaattactgccgaaatccagatgatgatgctcatggaccctggtgctacacgggaaatccactcattccttgggattattgccctatttctcgttgtgaaggtgataccacacctacaatagtcaatttagaccatcccgtaatatcttgtgccaaaacgaaacaattgcgagttgtaaatgggattccaacacgaacaaacataggatggatggttagtttgagatacagaaataaacatatctgcggaggatcattgataaaggagagttgggttcttactgcacgacagtgtttcccttctcgagacttgaaagattatgaagcttggcttggaattcatgatgtccacggaagaggagatgagaaatgcaaacaggttctcaatgtttcccagctggtatatggccctgaaggatcagatctggttttaatgaagcttgccaggcctgctgtcctggatgattttgttagtacgattgatttacctaattatggatgcacaattcctgaaaagaccagttgcagtgtttatggctggggctacactggattgatcaactatgatggcctattacgagtggcacatctctatataatgggaaatgagaaatgcagccagcatcatcgagggaaggtgactctgaatgagtctgaaatatgtgctggggctgaaaagattggatcaggaccatgtgagggggattatggtggcccacttgtttgtgagcaacataaaatgagaatggttcttggtgtcattgttcctggtcgtggatgtgccattccaaatcgtcctggtatttttgtccgagtagcatattatgcaaaatggatacacaaaattattttaacatataaggtaccacagtcagcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 68 - DNA, 1558 bp, Homo sapiens
atggagacagacacactcctgctatgggtactgctgctctgggttccaggttccactggtgacgcggcccagccggccggcagtgatcactttgcagcctccatgggtcagcgtgttccaagaggaaaccgtaaccttgcactgtgaggtgctccatctgcctgggagcagctctacacagtggtttctcaatggcacagccactcagacctcgacccccagctacagaatcacctctgccagtgtcaatgacagtggtgaatacaggtgccagagaggtctctcagggcgaagtgaccccatacagctggaaatccacagaggctggctactactgcaggtctccagcagagtcttcatggaaggagaacctctggccttgaggtgtcatgcgtggaaggataagctggtgtacaatgtgctttactatcgaaatggcaaagcctttaagtttttccactggaattctaacctcaccattctgaaaaccaacataagtcacaatggcacctaccattgctcaggcatgggaaagcatcgctacacatcagcaggaatatcacaatacactgtgaaagagctatttccagctccagtgctgaatgcatctgtgacatccccactcctggaggggaatctggtcaccctgagctgtgaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccggttaccaactcctgtctggtttcatgtcgcggccgcactcgagtctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggcggccctggcgggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcgctgagcacgaatttcgaggagggcccgaacaaaaactcatctcagaagaggatctgaatagcgccgtcgaccatcatcatcatcatcattga

SEQ ID NO: 69 - DNA, 1491 bp, Mus sp.; identical to SEQ ID NO: 3

SEQ ID NO: 70 - DNA, 1500 bp, Mus sp.
atggagacagacacactcctgctatgggtactgctgctctgggttccaggttccactggtgactctagaatggacaaagactgcgaaatgaagcgcaccaccctggatagccctctgggcaagctggaactgtctgggtgcgaacagggcctgcacgagatcaagctgctgggcaaaggaacatctgccgccgacgccgtggaagtgcctgccccagccgccgtgctgggcggaccagagccactgatgcaggccaccgcctggctcaacgcctactttcaccagcctgaggccatcgaggagttccctgtgccagccctgcaccacccagtgttccagcaggagagctttacccgccaggtgctgtggaaactgctgaaagtggtgaagttcggagaggtcatcagctaccagcagctggccgccctggccggcaatcccgccgccaccgccgccgtgaaaaccgccctgagcggaaatcccgtgcccattctgatcccctgccaccgggtggtgtctagctctggcgccgtggggggctacgagggcgggctcgccgtgaaagagtggctgctggcccacgagggccacagactgggcaagcctgggctgggcggatccaaactagctgagcacgaaggtgacgcggcccagccggccatggcccaggttcagcttcagcagtctggagctgagctgatgaagcctggggcctcagtgaagatatcctgcaaggctactggctacacattcaataacaactggatagagtgggtaaagcagaggcctggacatggccttgagtggattggagagattttacctggaagtggtagtactaagtacaatgagaagttcaagggcaaggccacattcactgcagatacatcctccaagacagcctacatgcaactcagcagcctgacatctgaggactctgccgtctattactgtacaagggtgtatggtaactacaatgctatggattactggggtcaaggaacctcggtcaccgtctcctcaggctccacctcaggctccggtaaacctggcccaggggagggatcaactaagggcgcgcctcaggttgttctcactcagtcatctgcactcaccacatcacctggtgaaacagtcacactcacttgtcgctcaagtactggggctgttacaactagtaactatgccaactgggtccaagaaaaaccagatcatttattcactggtctaataggtggtaccaacaaccgagctccaggtgttcctgccagattctcaggctccctgattggagacaaggctgccctcaccatcacaggggcacagactgaggatgaggcaatatatttctgtgctctatggtacagcaaccattgggtgttcggtggaggaaccaaagtgactgtcctaggccaggtcgacgcggccgcagggcccgaacaaaaactcatctcagaagaggatctgaatagcgccgtcgaccatcatcatcatcatcat